(19) United States
(12) Patent Application Publication
(10) Pub. No.: US 2026/0078161 A1
(43) Pub. Date: Mar. 19, 2026

(54) METHODS OF USING ACTIVIN RECEPTOR TYPE II VARIANTS

(71) Applicant: Keros Therapeutics, Inc., Lexington, MA (US)

(72) Inventors: Jasbir S. SEEHRA, Lexington, MA (US); Jennifer LACHEY, Lincoln, MA (US); Samuel M. CADENA, Arlington, MA (US)

(21) Appl. No.: 19/390,200

(22) Filed: Nov. 14, 2025

Related U.S. Application Data
(63) Continuation of application No. PCT/US2024/030006, filed on May 17, 2024.
(60) Provisional application No. 63/467,358, filed on May 18, 2023.

(51) Int. Cl. C07K 14/705; A61K 38/00

(52) U.S. Cl. CPC C07K 14/705; A61K 38/00; C07K 2319/30

(57) ABSTRACT

The invention features methods for the treatment of Alport syndrome using a polypeptide containing an extracellular ActRII variant, such as a polypeptide containing an extracellular ActRII chimera or an extracellular ActRIIB variant. The polypeptide may include an extracellular ActRII chimera or an extracellular ActRIIB variant fused to an Fc domain monomer. Treatment of Alport syndrome with a polypeptide described herein may reduce kidney inflammation, reduce kidney fibrosis, slow disease progression, and/or improve kidney function, which may reduce or delay the need for a kidney transplant or dialysis.

### SEQUENCE LISTING

The instant application contains a Sequence Listing which has been submitted electronically in XML file format and is hereby incorporated by reference in its entirety. Said XML copy, created on Nov. 12, 2025, is named 51184-051003_Sequence_Listing_11_12_25.xml and is 420,550 bytes in size.

### BACKGROUND OF THE INVENTION

Alport syndrome is a rare, genetic condition characterized by kidney disease, hearing loss, and eye abnormalities. It is caused by a mutation in a gene that encodes type 4 collagen and features kidney inflammation and fibrosis. In 80% of cases, Alport syndrome is inherited in an X-linked pattern and caused by COL4A5 mutations, although it can also be inherited in an autosomal recessive or autosomal dominant pattern due to mutations in COL4A3 or COL4A4. Prevalence is estimated to be less than 200,000 people in the United States. Eventually, subjects with Alport syndrome present with proteinuria, hypertension, progressive loss of kidney function (gradual decline in glomerular filtration rate), and end-stage renal disease. Currently, there is no specific treatment for Alport syndrome. Subjects with Alport syndrome may be treated with medications to treat symptoms or slow the progression of kidney disease, such as angiotensin converting enzyme inhibitors, angiotensin receptor blockers, sodium-glucose cotransporter-2 inhibitors, and diuretics. Although the treatment may delay the onset of renal impairment, most subjects with Alport syndrome will ultimately require dialysis or a kidney transplant. Accordingly, there exists a need for new treatments for Alport syndrome.

### SUMMARY OF THE INVENTION

The present invention features polypeptides that include an extracellular activin receptor type II (ActRII) variant, such as an extracellular ActRIIB variant or an extracellular ActRII chimera. In some embodiments, a polypeptide of the invention includes an extracellular ActRII variant fused to the N- or C-terminus of an Fc domain monomer (e.g., by fusion of the C-terminus of the ActRII variant to the N-terminus of an Fc domain monomer by way of a linker), which may be attached by amino acid or other covalent bonds and increase stability of the polypeptide. A polypeptide including an extracellular ActRII variant fused to an Fc domain monomer may also form a dimer (e.g., a homodimer or heterodimer) through the interaction between two Fc domain monomers. The polypeptides of the invention may be used to treat a subject having Alport syndrome (e.g., X-linked Alport syndrome, autosomal recessive Alport syndrome, or autosomal dominant Alport syndrome). The polypeptides described herein may treat Alport syndrome by reducing kidney fibrosis and/or kidney inflammation, by slowing or inhibiting the progression kidney fibrosis and/or kidney inflammation, or by delaying the development of kidney fibrosis and/or kidney inflammation. The polypeptides described herein may also improve kidney function, slow or inhibit a decline in kidney function, delay or prevent the onset of end stage renal disease, delay or prevent the need for dialysis, delay or prevent the need for continuous renal replacement therapy, or delay or prevent the need for a kidney transplant in a subject having Alport syndrome.

Exemplary embodiments of the invention are described in the enumerated paragraphs below.
E1. A method of treating a subject having Alport syndrome, the method comprising administering to the subject a therapeutically effective amount of a composition of Table 7 or a composition of Table 8.
E2. A method of reducing kidney fibrosis, delaying the development of kidney fibrosis, or slowing or inhibiting the progression of kidney fibrosis in a subject having Alport syndrome, the method comprising administering to the subject a therapeutically effective amount of a composition of Table 7 or a composition of Table 8.
E3. A method of reducing kidney inflammation or slowing or inhibiting the progression of kidney inflammation in a subject having Alport syndrome, the method comprising administering to the subject a therapeutically effective amount of a composition of Table 7 or a composition of Table 8.
E4. A method of improving kidney function or slowing or inhibiting a decline in kidney function in a subject having Alport syndrome, the method comprising administering to the subject a therapeutically effective amount of a composition of Table 7 or a composition of Table 8.
E5. The method of E4, wherein improving kidney function or slowing or inhibiting a decline in kidney function results in improved glomerular filtration rate (GFR) or slowing or stopping a progressive decline in GFR, improved protein-to-creatinine ratio (PCR), albumin-to-creatinine ratio (ACR), or urinary albumin-creatinine-ratio (UACR) or slowing or stopping a progressive increase in PCR, ACR or UACR, reduced blood urea nitrogen, reduced creatinine in the blood, improved creatinine clearance, or reduced proteinuria.
E6. A method of delaying or inhibiting the onset of end-stage renal disease in a subject having Alport syndrome, comprising administering to the subject a therapeutically effective amount of a composition of Table 7 or a composition of Table 8.
E7. A method of improving GFR (or eGFR) or preventing, delaying the onset of, slowing or inhibiting the progression of, or reducing the risk of a decline in GFR (or eGFR) in a subject having Alport syndrome, comprising administering to the subject a therapeutically effective amount of a composition of Table 7 or a composition of Table 8.
E8. A method of reducing glomerulosclerosis, delaying the development of glomerulosclerosis, or slowing or inhibiting the progression glomerulosclerosis in a subject having Alport syndrome, comprising administering to the subject a therapeutically effective amount of a composition of Table 7 or a composition of Table 8.
E9. A method of treating, preventing, delaying the onset of, slowing the progression of, or reducing the risk of hematuria, proteinuria, or albuminuria in a subject having Alport syndrome, comprising administering to the subject a therapeutically effective amount of a composition of Table 7 or a composition of Table 8.
E10. The method of any one of E1-E9, wherein the subject has X-linked Alport syndrome.
E11. The method of any one of E1-E9, wherein the subject has autosomal recessive Alport syndrome.
E12. The method of any one of E1-E9, wherein the subject has autosomal dominant Alport syndrome.
E13. The method of any one of E1-E2, wherein the subject has a mutation in COL4A3, a mutation in COL4A4, a mutation in COL4A5, or a mutation in both COL4A3 and COL4A4.
E14. The method of any one of E1-E13, wherein the subject is male.
E15. The method of any one of E1-E13, wherein the subject is female.
E16. The method of any one of E1-E15, wherein the method reduces kidney fibrosis or slows or inhibits progression of kidney fibrosis.
E17. The method of any one of E1-E16, wherein the method reduces kidney inflammation or slows or inhibits progression of kidney inflammation.
E18. The method of any one of E1-E17, wherein the method improves kidney function or slows or inhibits a decline in kidney function.
E19. The method of any one of E1-E18, wherein the method improves GFR or slows or inhibits a decline in GFR.
E20. The method of any one of E1-E19, wherein the method reduces hematuria, proteinuria, albuminuria, blood urea nitrogen, or creatinine in the blood.
E21. The method of any one of E1-E20, wherein the method delays or prevents the onset of end stage renal disease.
E22. The method of any one of E1-E21, wherein the method delays or prevents a need for dialysis, continuous renal replacement therapy, or a kidney transplant.
E23. The method of any one of E1-E22, wherein the method improves life expectancy for the subject.
E24. The method of any one of E1-E23, wherein the method comprises administering to the subject a therapeutically effective amount of a composition of Table 7.
E25. The method of any one of E1-E24, wherein the composition of Table 7 is polypeptide comprising an extracellular ActRII chimera having a sequence of any one of SEQ ID NOs: 1-21, wherein X₁ is D or R, X₂ is I, F, E, D, Y, S, N, Q, or T, X₃ is N or T, X₄ is A or E, X₅ is T or K, X₆ is E or K, X₇ is E or D, X₈ is N or S, and X₉ is Q, E, K, R, D, or N, optionally wherein the chimera is truncated from the N-terminus by deletion of 1, 2, 3, 4, 5, 6, 7, 8, or 9 amino acids, wherein the chimera retains the two amino acids before the first cysteine.
E26. The method of E25, wherein the chimera has the sequence of SEQ ID NO: 19.
E27. The method of E25, wherein the chimera has the sequence of SEQ ID NO: 12.
E28. The method of E25, wherein the chimera has the sequence of SEQ ID NO: 5.
E29. The method of any one of E25-E28, wherein X₁ is D, X₂ is I, F, or E, X₃ is N or T, X₄ is A or E, X₅ is T or K, X₆ is E or K, X₇ is E or D, X₈ is N or S, and X₉ is E or Q.
E30. The method of E29, wherein X₁ is D, X₂ is I or F, X₃ is N, X₄ is A or E, X₅ is T or K, X₆ is E or K, X₇ is E or D, X₈ is N or S, and X₉ is E or Q.
E31. The method of E30, wherein X₁ is D, X₂ is F, X₃ is N, X₄ is E, X₅ is K, X₆ is K, X₇ is D, X₈ is S, and X₉ is Q.
E32. The method of E25, wherein the chimera has the sequence of any one of SEQ ID NOs: 22-43.
E33. The method of E32, wherein the chimera has the sequence of SEQ ID NO: 41.
E34. The method of E32, wherein the chimera has the sequence of SEQ ID NO: 25.
E35. The method of E32, wherein the chimera has the sequence of SEQ ID NO: 40.
E36. The method of any one of E25-E35, wherein the polypeptide further includes an Fc domain monomer fused to the C-terminus of the polypeptide (e.g., the C-terminus of the chimera) by way of a linker.
E37. The method of E36, wherein the polypeptide is in the form of a dimer (e.g., a homodimer).
E38. The method of E36, wherein the polypeptide has the sequence of any one of SEQ ID NOs: 107-110 and SEQ ID NOs: 184-263.
E39. The method of E38, wherein the polypeptide has the sequence of SEQ ID NO: 216.
E40. The method of E38, wherein the polypeptide has the sequence of SEQ ID NO: 110.
E41. The method of E38, wherein the polypeptide has the sequence of SEQ ID NO: 215.
E42. The method of any one of E38-E41, wherein the polypeptide is in the form of a homodimer.
E43. The method of any one of E1-E23, wherein the method comprises administering to the subject a therapeutically effective amount of a composition of Table 8.
E44. The method of any one of E1-E23 and E43, wherein the composition of Table 8 is a polypeptide comprising an extracellular activin receptor type IIB (ActRIIB) variant, the variant having one or more amino acid substitutions relative to the sequence of GRGEAETRECIYYNANWELERTNQSGLERCEGEQDKRLHCYASWRNSSGTIELVKKGCWLDDF NCYDRQECVATEENPQVYFCCCEGNFCNERFTHLPEAGGPEVTYEPPPTAPT (SEQ ID NO: 45), wherein the variant comprises one or more amino acid substitutions that impart reduced BMP9 binding relative to wild type extracellular ActRIIB and one or more additional amino acid substitutions, wherein the substitutions that reduce BMP9 binding comprise one or more of:
   a) amino acid substitution E75K;
   b) amino acid substitutions Q69T and E70D; or
   c) amino acid substitutions Q69D and E70T, optionally wherein the variant is truncated from the N-terminus by deletion of 1, 2, 3, 4, 5, 6, or 7 amino acids.
E45. The method of E44, wherein the variant comprises amino acid substitutions I11L, L27V, Q34K, T50S, I51L, L531, Q69D, E70T, E75K, and F89M or amino acid substitutions I11L, L27V, Q34K, T50S, I51L, L531, Q69T, E70D, E75K, and F89M.
E46. The method of E44, wherein the variant has the sequence of any one of SEQ ID NOs: 270-283.
E47. The method of E46, wherein the variant has the sequence of SEQ ID NO: 283.
E48. The method of any one of E44-E47, wherein the polypeptide further includes an Fc domain monomer fused to the C-terminus of the polypeptide (e.g., the C-terminus of the variant) by way of a linker.
E49. The method of E48, wherein the polypeptide is in the form of a dimer (e.g., a homodimer).
E50. The method of E48, wherein the polypeptide has the sequence of SEQ ID NO: 284.
E51. The method of E50, wherein the polypeptide is in the form of a homodimer.
E52. The method of any one of E1-E51, wherein the method further comprises administering to the subject an additional therapeutic agent.
E53. The method of E52, wherein the additional therapeutic agent is an angiotensin II converting enzyme inhibitor (ACE inhibitor), angiotensin II receptor blocker (ARB), beta-blocker, diuretic, angiotensin receptor-neprilysin inhibitor (ARNi), calcium channel blocker, sodium-glucose cotransporter-2 (SGLT-2) inhibitor, ivabradine, HMG-CoA reductase inhibitor, aldosterone inhibitor, aliskiren, calcineurin inhibitor, endothelin receptor antagonist, sulodexide, vasopeptidase inhibitor, anti-transforming growth factor-β1 antibody, chemokine receptor 1 blocker, bone morphogenetic protein-7, PPARy agonist, or matrix metalloproteinase inhibitor.
E54. The method of any one of E1-E53, wherein the composition is administered in an amount sufficient to reduce kidney fibrosis, delay the development of kidney fibrosis, slow or inhibit the progression of kidney fibrosis, reduce kidney inflammation, slow or inhibit the progression of kidney inflammation, improve kidney function, slow or inhibit a decline in kidney function, improve glomerular filtration rate (GFR), slow or stop a progressive decline in GFR, improve PCR, ACR, or UACR, slow or stop a progressive increase in PCR, ACR or UACR, reduce blood urea nitrogen, reduce creatinine in the blood, improve creatinine clearance, reduce proteinuria, delay or inhibit the onset of end-stage renal disease, reduce glomerulosclerosis, delay the development of glomerulosclerosis, slow or inhibit the progression glomerulosclerosis, treat, prevent, delay the onset of, slow the progression of, or reduce the risk of hematuria, proteinuria, delay or prevent a need for dialysis, continuous renal replacement therapy, or a kidney transplant, or improve life expectancy.
E55. The method of any one of E1-E54, wherein the method does not cause a vascular complication in the subject.
E56. The method of E55, wherein the method does not increase vascular permeability or leakage.
E57. The method of any one of E1-E56, wherein the subject is a human.

### Definitions

To facilitate the understanding of this invention, a number of terms are defined below. Terms defined herein have meanings as commonly understood by a person of ordinary skill in the areas relevant to the invention. Terms such as “a,” “an,” and “the” are not intended to refer to only a singular entity but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the terms “extracellular activin receptor type II (ActRII) chimera,” “extracellular ActRII chimera,” and “ActRII chimera” refer to a polypeptide including a soluble, extracellular portion of the single transmembrane receptor ActRIIB and a soluble, extracellular portion of the single transmembrane receptor ActRIIA. The ActRII chimeras described herein result from joining an N-terminal portion of extracellular ActRIIB to a C-terminal portion of extracellular ActRIIA such that the sequences are contiguous (e.g., the ActRIIA sequence continues where the ActRIIB sequence left off, starting with the next the amino acid located in the corresponding position of ActRIIA). The extracellular ActRII chimera may also include one or more amino acid substitutions in the portion of the chimera that corresponds to the sequence of ActRIIB compared to a wild-type extracellular ActRIIB (e.g., bold portion of the sequence of SEQ ID NO: 46 shown below) and/or one or more amino acid substitutions in the portion of the chimera that corresponds to the sequence of ActRIIA compared to a wild-type extracellular ActRIIA (e.g., bold portion of the sequence of SEQ ID NO: 47 shown below). The extracellular ActRII chimera may also have an N-terminal truncation of 1-9 amino acids relative to the extracellular portion of ActRIIB or ActRIIA. The sequences of wild-type, human ActRIIB (SEQ ID NO: 46) and wild-type, human ActRIIA (SEQ ID NO: 47) are shown below, in which the signal peptide is italicized and the extracellular portion is bold.

Wild-type human ActRIIB (SEQ ID NO: 46):
MTAPWVALALLWGSLCAGSGRGEAETRECIYYNANWELERTNQSGLERCEGEQDKRLHCYAS
WRNSSGTIELVKKGCWLDDFNCYDRQECVATEENPQVYFCCCEGNFCNERFTHLPEAGGPEV
TYEPPPTAPTLLTVLAYSLLPIGGLSLIVLLAFWMYRHRKPPYGHVDIHEDPGPPP
PSPLVGLKPLQLLEIKARGRFGCVWKAQLMNDFVAVKIFPLQDKQSWQSEREIFSTPGMK
HENLLQFIAAEKRGSNLEVELWLITAFHDKGSLTDYLKGNIITWNELCHVAETMSRGLSY
LHEDVPWCRGEGHKPSIAHRDFKSKNVLLKSDLTAVLADFGLAVRFEPGKPPGDTHGQVG
TRRYMAPEVLEGAINFQRDAFLRIDMYAMGLVLWELVSRCKAADGPVDEYMLPFEEEIGQ
HPSLEELQEVVVHKKMRPTIKDHWLKHPGLAQLCVTIEECWDHDAEARLSAGCVEERVSL
IRRSVNGTTSDCLVSLVTSVTNVDLPPKESSI
Wild-type, human ActRIIA precursor protein (SEQ ID NO: 47):
MGAAAKLAFAVFLISCSSGAILGRSETQECLFFNANWEKDRTNQTGVEPCYGDKDKRRHCFAT
WKNISGSIEIVKQGCWLDDINCYDRTDCVEKKDSPEVYFCCCEGNMCNEKFSYFPEMEVTQPT
SNPVTPKPPYYNILLYSLVPLMLIAGIVICAFWVYRHHKMAYPPVLVPTQDPGPPPPSPLLGLKPL
QLLEVKARGRFGCVWKAQLLNEYVAVKIFPIQDKQSWQNEYEVYSLPGMKHENILQFIGAEKRG
TSVDVDLWLITAFHEKGSLSDFLKANVVSWNELCHIAETMARGLAYLHEDIPGLKDGHKPAISHR
DIKSKNVLLKNNLTACIADFGLALKFEAGKSAGDTHGQVGTRRYMAPEVLEGAINFQRDAFLRID
MYAMGLVLWELASRCTAADGPVDEYMLPFEEEIGQHPSLEDMQEVVVHKKKRPVLRDYWQKH
AGMAMLCETIEECWDHDAEARLSAGCVGERITQMQRLTNIITTEDIVTVVTMVTNVDFPPKESSL

An extracellular ActRII chimera may have the sequence of any one of SEQ ID NOs: 1-43. In particular embodiments, an extracellular ActRII chimera has the sequence of any one of SEQ ID NOs: 22-43 (Table 2).

As used herein, the terms “extracellular activin receptor type IIB (ActRIIB) variant” and “ActRIIB variant” refer to a polypeptide including a soluble, extracellular portion of the single transmembrane receptor, ActRIIB, that has at least one amino acid substitution relative to a wild-type extracellular ActRIIB (e.g., bold portion of the sequence of SEQ ID NO: 46, shown above). An extracellular ActRIIB variant may have a sequence of any one of SEQ ID NOs: 269-283. In particular embodiments, an extracellular ActRIIB variant has a sequence of any one of SEQ ID NOs: 270-283 (Table 6). In some embodiments, an extracellular ActRIIB variant may have at least 85% (e.g., at least 85%, 87%, 90%, 92%, 95%, 96%, 97%, 98%, 99%, or greater) amino acid sequence identity to the sequence of a wild-type extracellular ActRIIB (SEQ ID NO: 45). The extracellular ActRIIB variant may also have an N-terminal truncation of 1-7 amino acids relative to the extracellular portion of ActRIIB.

As used herein, the term “extracellular activin receptor type II (ActRII) variant” refers to an extracellular ActRIIB variant or an extracellular ActRII chimera described herein.

As used herein, the term “N-terminal truncation” refers to a deletion of 1-7 amino acids (e.g., 1, 2, 3, 4, 5, 6, or 7 amino acids) from the N-terminus of an extracellular ActRIIB variant (e.g., an extracellular ActRIIB variant having the sequence of any one of SEQ ID NOs: 269-283 (e.g., SEQ ID NOs: 270-283)) or a deletion of 1-9 amino acids (e.g., 1, 2, 3, 4, 5, 6, 7, 8, or 9 amino acids) from the N-terminus of an extracellular ActRII chimera (e.g., an extracellular ActRII chimera having the sequence of any one of SEQ ID NOs: 1-43 (e.g., SEQ ID NOs: 22-43)). The N-terminal truncation can remove amino acids up to two amino acids before the first cysteine (e.g., the two amino acids before the first cysteine (RE) are retained in an N-terminally truncated extracellular ActRIIB variant and the two amino acids before the first cystine (RE or QE) are retained in an N-terminally truncated extracellular ActRII chimera).

As used herein, the term “linker” refers to a linkage between two elements, e.g., peptides or protein domains. A polypeptide described herein may include an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) fused to an Fc domain monomer, which increases stability or improves pharmacokinetic properties of the polypeptide. The Fc domain monomer may be fused to the polypeptide by way of a linker. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 1-200 amino acid sequence) occurring between two elements, e.g., peptides or protein domains, to provide space and/or flexibility between the two elements. An amino acid spacer is part of the primary sequence of a polypeptide (e.g., fused to the spaced peptides via the polypeptide backbone). The formation of disulfide bonds, e.g., between two hinge regions that form an Fc domain, is not considered a linker.

As used herein, the term “Fc domain” refers to a dimer of two Fc domain monomers. An Fc domain has at least 80% sequence identity (e.g., at least 85%, 90%, 95%, 97%, or 100% sequence identity) to a human Fc domain that includes at least a C_{H}2 domain and a C_{H}3 domain. An Fc domain monomer includes second and third antibody constant domains (C_{H}2 and C_{H}3). In some embodiments, the Fc domain monomer also includes a hinge domain. An Fc domain does not include any portion of an immunoglobulin that is capable of acting as an antigen-recognition region, e.g., a variable domain or a complementarity determining region (CDR). In the wild-type Fc domain, the two Fc domain monomers dimerize by the interaction between the two C_{H}3 antibody constant domains, as well as one or more disulfide bonds that form between the hinge domains of the two dimerizing Fc domain monomers. In some embodiments, an Fc domain may be mutated to lack effector functions, typical of a “dead Fc domain.” In certain embodiments, each of the Fc domain monomers in an Fc domain includes amino acid substitutions in the C_{H}2 antibody constant domain to reduce the interaction or binding between the Fc domain and an Fcγ receptor. In some embodiments, the Fc domain contains one or more amino acid substitutions that reduce or inhibit Fc domain dimerization. An Fc domain can be any immunoglobulin antibody isotype, including IgG, IgE, IgM, IgA, or IgD. Additionally, an Fc domain can be an IgG subtype (e.g., IgG1, IgG2a, IgG2b, IgG3, or IgG4). The Fc domain can also be a non-naturally occurring Fc domain, e.g., a recombinant Fc domain.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell, e.g., a human red blood cell, platelet, neutrophil, or muscle cell).

As used herein, the term “fused” is used to describe the combination or attachment of two or more elements, components, or protein domains, e.g., peptides or polypeptides, by means including chemical conjugation, recombinant means, and chemical bonds, e.g., amide bonds. For example, two single peptides in tandem series can be fused to form one contiguous protein structure, e.g., a polypeptide, through chemical conjugation, a chemical bond, a peptide linker, or any other means of covalent linkage. In some embodiments of a polypeptide described herein, an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) may be fused in tandem series to the N- or C-terminus of an Fc domain monomer (e.g., the sequence of SEQ ID NO: 48, SEQ ID NO: 100, or SEQ ID NO: 264) by way of a linker. For example, an extracellular ActRII variant is fused to an Fc domain monomer by way of a peptide linker, in which the N-terminus of the peptide linker is fused to the C-terminus of the extracellular ActRII variant through a chemical bond, e.g., a peptide bond, and the C-terminus of the peptide linker is fused to the N-terminus of the Fc domain monomer through a chemical bond, e.g., a peptide bond.

As used herein, the terms “increasing” and “decreasing” refer to modulating resulting in, respectively, greater or lesser amounts, of function, expression, or activity of a metric relative to a reference. For example, subsequent to administration of a polypeptide described herein including an extracellular ActRII variant in a method described herein, the amount of a marker of a metric (e.g., kidney fibrosis) as described herein may be increased or decreased in a subject relative to the amount of the marker prior to administration. Generally, the metric is measured subsequent to administration at a time that the administration has had the recited effect, e.g., at least one week, one month, 3 months, or 6 months, after a treatment regimen has begun.

As used herein, the term “fibrosis” refers to the pathological process of formation of excess fibrous connective tissue in an organ or tissue. Fibrosis is characterized by fibroblast accumulation and collagen deposition in excess of normal deposition in any particular tissue. In response to inflammation or an injury to a tissue, nearby fibroblasts can migrate into the wound, proliferate, and produce large amounts of collagenous extracellular matrix. When fibrosis occurs in response to injury, the term “scarring” can be used as synonym.

As used herein, the term “Alport syndrome” refers to an inherited form of kidney disease in which an abnormal level of glomerular basement membrane (GBM) is produced, leading to interstitial fibrosis, glomerular sclerosis, and eventual loss of kidney function. The disease is also frequently characterized by hearing defects and ocular anomalies. Alport syndrome is characterized by one or more mutations that impair the production, deposition, or function of the collagen IV alpha345 network, such as one or more mutations in COL4A5, which encodes the collagen IV alpha 5 chain, one or more mutations in COL4A3, which encodes the collagen IV alpha 3 chain, one or more mutations in COL4A4, which encodes the collagen IV alpha 4 chain, or one or more mutations in COL4A3 and COL4A4. Alport syndrome may be X-linked, autosomal dominant or autosomal recessive.

As used herein, the term “albuminuria” refers to a condition in which more than the normal amount of albumin is present in the urine. Normally, the glomerular filtration permeability barrier, which is composed of podocytes, glomerular basement membrane, and endothelial cells, prevents serum protein from leaking into urine. Albuminuria may reflect injury of the glomerular filtration permeability barrier. Albuminuria may be calculated from a 24-hour urine sample, an overnight urine sample or a spot-urine sample. Albuminuria can be determined by the albumin excretion rate (AER) and/or the albumin-to-creatine ratio (ACR) in the urine (also referred to as UACR). “Albumin/creatinine ratio” refers to the ratio of urine albumin (mg/dL) per urine creatinine (g/dL) and is expressed as mg/g. In certain embodiments, albumin/creatinine ratio may be calculated from a spot-urine sample and may be used as an estimate of albumin excretion over a 24-hour period.

As used herein, the term “blood urea nitrogen” refers to a measure of the amount of nitrogen in the blood in the form of urea. The liver produces urea in the urea cycle as a waste product of the digestion of protein, and the urea is removed from the blood by the kidneys. Normal human adult blood may contain between 7 to 21 mg of urea nitrogen per 100 ml (7-21 mg/dL) of blood. Measurement of blood urea nitrogen is used as an indicator of renal health. If the kidneys are not able to remove urea from the blood normally, a subject's blood urea nitrogen rises.

As used herein, the term “end stage renal disease (ESRD)” refers to the complete or almost complete failure of kidney function.

As used herein the term “hematuria” refers to a condition characterized by the presence of red blood cells in the urine.

As used herein, the terms “estimated glomerular filtration rate (eGFR)” and “glomerular filtration rate (GFR)” refer to a measurement of how well the kidneys are filtering creatinine and are used as an estimate of how much blood passes through the glomeruli per minute. The estimated GFR may be calculated based on serum creatinine values, e.g., using the Chronic Kidney Disease Epidemiology Collaboration (CKD-EPI) equation, the Cockcroft-Gault formula, or the Modification of Diet in Renal Disease (MDRD) formula, which are all known in the art. Normal results may range from 90-120 mL/min/1.73 m². Values of >60 to <90 mL/min/1.73 m² may indicate mild renal impairment, values of >30 to <60 mL/min/1.73 m² may indicate moderate renal impairment, and values of >15 to <30 mL/min/1.73 m² may indicate severe renal impairment. Levels below 60 mL/min/1.73 m² for three or more months may be an indicator of chronic kidney disease. Levels below 15 mL/min/1.73 m² may be an indicator of kidney failure.

As used herein the term “proteinuria” refers to a condition in which there is a presence of excess protein in the urine. The protein content in urine is usually measured as a concentration in mg/dL. The concentration of protein in the urine may be compared to the creatinine level in the urine sample. This is termed the protein/creatinine ratio (PCR). Usually, proteinuria is defined as a protein/creatinine ratio (PCR) greater than 45 mg/mmol, which is equivalent to an albumin-to-creatinine ration (ACR) of greater than 30 mg/mmol or approximately 300 mg/g. Proteinuria may also be characterized by the excretion of >250 mg of protein into the urine per 24 hours and/or a urine protein to creatinine ratio of >0.20 mg/mg.

As used herein, the term “C-terminal extension” refers to the addition of one or more amino acids to the C-terminus of a an extracellular ActRII chimera (e.g., an extracellular ActRII chimera having the sequence of any one of SEQ ID NOs: 1-43 (e.g., SEQ ID NOs: 22-43)). The C-terminal extension can be one or more amino acids, such as 1-6 amino acids (e.g., 1, 2, 3, 4, 5, 6 or more amino acids). The C-terminal extension may include amino acids from the corresponding position of wild-type ActRIIA or ActRIIB. Exemplary C-terminal extensions are the amino acid sequence NP (a two amino acid C-terminal extension) and the amino acid sequence NPVTPK (SEQ ID NO: 104) (a six amino acid C-terminal extension). Any amino acid sequence that does not disrupt the activity of the polypeptide can be used.

As used herein, the term “percent (%) identity” refers to the percentage of amino acid (or nucleic acid) residues of a candidate sequence, e.g., an extracellular ActRIIA variant, an extracellular ActRIIB variant, or an extracellular ActRII chimera, that are identical to the amino acid (or nucleic acid) residues of a reference sequence, e.g., a wild-type extracellular ActRIIA (e.g., SEQ ID NO: 44) or wild-type extracellular ActRIIB (e.g., SEQ ID NO: 45), after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid (or nucleic acid) sequence identity of a given candidate sequence to, with, or against a given reference sequence (which can alternatively be phrased as a given candidate sequence that has or includes a certain percent amino acid (or nucleic acid) sequence identity to, with, or against a given reference sequence) is calculated as follows:

$$100 \times ( fraction of A / B )$$

where A is the number of amino acid (or nucleic acid) residues scored as identical in the alignment of the candidate sequence and the reference sequence, and where B is the total number of amino acid (or nucleic acid) residues in the reference sequence. In some embodiments where the length of the candidate sequence does not equal to the length of the reference sequence, the percent amino acid (or nucleic acid) sequence identity of the candidate sequence to the reference sequence would not equal to the percent amino acid (or nucleic acid) sequence identity of the reference sequence to the candidate sequence.

In particular embodiments, a reference sequence aligned for comparison with a candidate sequence may show that the candidate sequence exhibits from 50% to 100% identity across the full length of the candidate sequence or a selected portion of contiguous amino acid (or nucleic acid) residues of the candidate sequence. The length of the candidate sequence aligned for comparison purpose is at least 30%, e.g., at least 40%, e.g., at least 50%, 60%, 70%, 80%, 90%, or 100% of the length of the reference sequence. When a position in the candidate sequence is occupied by the same amino acid (or nucleic acid) residue as the corresponding position in the reference sequence, then the molecules are identical at that position.

As used herein, the term “serum half-life” refers to, in the context of administering a therapeutic protein to a subject, the time required for plasma concentration of the protein in the subject to be reduced by half. The protein can be redistributed or cleared from the bloodstream, or degraded, e.g., by proteolysis. Serum half-life comparisons can be made by comparing the serum half-life of Fc fusion proteins.

As used herein, the terms “affinity” and “binding affinity” refer to the strength of the binding interaction between two molecules. Generally, binding affinity refers to the strength of the sum total of non-covalent interactions between a molecule and its binding partner, such as an extracellular ActRII chimera or an extracellular ActRIIB variant and BMP9 or activin A. Unless indicated otherwise, binding affinity refers to intrinsic binding affinity, which reflects a 1:1 interaction between members of a binding pair. The binding affinity between two molecules is commonly described by the dissociation constant (K_{D}) or the affinity constant (K_{A}). Two molecules that have low binding affinity for each other generally bind slowly, tend to dissociate easily, and exhibit a large K_{D}. Two molecules that have high affinity for each other generally bind readily, tend to remain bound longer, and exhibit a small K_{D}. The K_{D} of two interacting molecules may be determined using methods and techniques well known in the art, e.g., surface plasmon resonance. K_{D} is calculated as the ratio of k_{off}/kₒₙ.

As used herein, the phrase “affecting myostatin, activin A, activin B, and/or BMP9 signaling” means changing the binding of myostatin, activin A, activin B, and/or BMP9 to their receptors, e.g., ActRIIA, ActRIIB, and BMPRII (e.g., ActRIIA or ActRIIB, e.g., endogenous ActRIIA or ActRIIB). In some embodiments, a polypeptide including an extracellular ActRII chimera or an extracellular ActRIIB variant described herein reduces or inhibits the binding of myostatin, activin A, activin B, and/or BMP9 to their receptors, e.g., ActRIIA, ActRIIB, and/or BMPRII (e.g., ActRIIA or ActRIIB, e.g., endogenous ActRIIA or ActRIIB).

As used herein, the term “vascular complication” refers to a vascular disorder or any damage to the blood vessels, such as damage to the blood vessel walls. Damage to the blood vessel walls may cause an increase in vascular permeability or leakage. The term “vascular permeability or leakage” refers to the capacity of the blood vessel walls to allow the flow of small molecules, proteins, and cells in and out of blood vessels. An increase in vascular permeability or leakage may be caused by an increase in the gaps (e.g., an increase in the size and/or number of the gaps) between endothelial cells that line the blood vessel walls and/or thinning of the blood vessel walls.

As used herein, the term “polypeptide” describes a single polymer in which the monomers are amino acid residues which are covalently conjugated together through amide bonds. A polypeptide is intended to encompass any amino acid sequence, either naturally occurring, recombinant, or synthetically produced.

As used herein, the term “homodimer” refers to a molecular construct formed by two identical macromolecules, such as proteins or nucleic acids. The two identical monomers may form a homodimer by covalent bonds or non-covalent bonds. For example, an Fc domain may be a homodimer of two Fc domain monomers if the two Fc domain monomers contain the same sequence. In another example, a polypeptide described herein including an extracellular ActRII chimera or an extracellular ActRIIB variant fused to an Fc domain monomer may form a homodimer through the interaction of two Fc domain monomers, which form an Fc domain in the homodimer.

As used herein, the term “heterodimer” refers to a molecular construct formed by two different macromolecules, such as proteins or nucleic acids. The two monomers may form a heterodimer by covalent bonds or non-covalent bonds. For example, a polypeptide described herein including an extracellular ActRII chimera or an extracellular ActRIIB variant fused to an Fc domain monomer may form a heterodimer through the interaction of two Fc domain monomers, each fused to a different ActRII chimera or ActRIIB variant, which form an Fc domain in the heterodimer (e.g., a heterodimer can contain two different extracellular ActRIIB variants or two different extracellular ActRII chimeras).

As used herein, the term “host cell” refers to a vehicle that includes the necessary cellular components, e.g., organelles, needed to express proteins from their corresponding nucleic acids. The nucleic acids are typically included in nucleic acid vectors that can be introduced into the host cell by conventional techniques known in the art (transformation, transfection, electroporation, calcium phosphate precipitation, direct microinjection, etc.). A host cell may be a prokaryotic cell, e.g., a bacterial cell, or a eukaryotic cell, e.g., a mammalian cell (e.g., a CHO cell or a HEK293 cell).

As used herein, the term “therapeutically effective amount” refers an amount of a polypeptide, nucleic acid, or vector described herein or a pharmaceutical composition containing a polypeptide, nucleic acid, or vector described herein effective in achieving the desired therapeutic effect in treating a patient having a or at risk of developing a disease, such as Alport syndrome. In particular, the therapeutically effective amount of the polypeptide, nucleic acid, or vector avoids adverse side effects.

As used herein, the term “pharmaceutical composition” refers to a medicinal or pharmaceutical formulation that includes an active ingredient as well as excipients and diluents to enable the active ingredient suitable for the method of administration. The pharmaceutical composition of the present invention includes pharmaceutically acceptable components that are compatible with the polypeptide, nucleic acid, or vector. The pharmaceutical composition may be in tablet or capsule form for oral administration or in aqueous form for intravenous or subcutaneous administration.

As used herein, the term “pharmaceutically acceptable carrier or excipient” refers to an excipient or diluent in a pharmaceutical composition. The pharmaceutically acceptable carrier must be compatible with the other ingredients of the formulation and not deleterious to the recipient. In the present invention, the pharmaceutically acceptable carrier or excipient must provide adequate pharmaceutical stability to the polypeptide including an extracellular ActRII chimera or an extracellular ActRIIB variant, the nucleic acid molecule(s) encoding the polypeptide, or a vector containing such nucleic acid molecule(s). The nature of the carrier or excipient differs with the mode of administration. For example, for intravenous administration, an aqueous solution carrier is generally used; for oral administration, a solid carrier is preferred.

As used herein, “treatment” and “treating” in reference to a disease or condition, refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to have the condition or disorder or those in which the condition or disorder is to be prevented.

As used herein, the term “subject” refers to a mammal, e.g., preferably a human. Mammals include, but are not limited to, humans and domestic and farm animals, such as monkeys (e.g., a cynomolgus monkey), mice, rats, dogs, cats, horses, sheep, goats, rabbits, and cows, etc.

### BRIEF DESCRIPTION OF THE DRAWINGS

FIG. 1 is a heat map showing changes in serum proteins in healthy postmenopausal women administered a single dose of ActRIIB 2.12-Fc at 4.5 mg/kg. Administration of ActRIIB 2.12-Fc altered serum proteins associated with inflammation and extracellular matrix remodeling pathways.

FIGS. 2A-2J are a series of graphs showing representative examples of serum proteins altered by ActRIIB 2.12-Fc administration in post-menopausal women. ActRIIB 2.12-Fc decreased serum collagen fragments (FIGS. 2A-2B), fibrosis markers (FIGS. 2C-2D), and pro-inflammatory cytokines (FIGS. 2E-2F) and increased anti-inflammatory cytokines (FIGS. 2G-2H) and markers of macrophage repolarization (FIGS. 2I-2J). Data are represented as mean fold change from baseline±SEM, N=5-6 per group. COL2A1=Collagen type II; COL3A1=Collagen Type III; IL=Interleukin; MMP-7=matrix metalloproteinase-7; MMP10=matrix metalloproteinase-10; MARCO=Macrophage Receptor with Collagenous Structure; sCD163=soluble cluster of differentiation 163.

FIG. 3 is a graph showing blood urea nitrogen (BUN) measurements during a 12-week treatment regimen of 4-week old B6129F1/J Col4a3^{-/-} mice with either vehicle (n=6), ramipril (n=4), Chimera 1/2b-mFc (a homodimer of SEQ ID NO: 41 fused to a mouse Fc domain monomer by a GGG linker, n=7), or Chimera 1/2b-mFc in combination with ramipril (n=4). The mice started treatment at 4 weeks of age and ended treatment at 16 weeks of age. Mouse age is shown on the x-axis. Data are shown as mean±SEM with BUN in mg/dL.

FIG. 4 is a graph showing albumin creatine ratios (ACR) measured during a 12-week treatment regimen of 4-week-old B6129F1/J Col4a3^{-/-} mice with either vehicle (n=6), ramipril (n=4), Chimera 1/2b-mFc (n=7), or Chimera 1/2b-mFc in combination with ramipril (n=4). The mice started treatment at 4 weeks of age and ended treatment at 16 weeks of age. Mouse age is shown on the x-axis. Data are shown as mean±SEM with urinary albumin/creatinine ratios (ACR) in g/mg. Statistics are shown using 2-way ANOVA with a Tukey's multiple comparison test. *P<0.05, **P<0.01, ***P<0.001.

FIGS. 5A-5D are a series of graphs showing trichrome staining of UUO kidneys after a treatment regimen of 7-8-week-old male C57BL/6 mice with either IgG2a (n=6), ActRIIA-mFc (a homodimer of a human ActRIIA extracellular domain (SEQ ID NO: 44) fused to a mouse Fc domain monomer by way of a GGG linker, n=6), or ActRIIB-mFc (a homodimer of a human ActRIIB extracellular domain (SEQ ID NO: 45) fused to a mouse Fc domain by way of a GGG linker, n=6). Data are shown as mean±SEM. Statistics are shown using ANOVA with a Tukey's post hoc test. **P<0.01.

FIGS. 6A-6D are a series of graphs showing picrosirius red (PSR) staining of UUO kidneys after a treatment regimen of 7-8-week-old male C57BL/6 mice with either IgG2a (n=6), ActRIIA-mFc (n=6), or ActRIIB-mFc (n=6). Data are shown as mean±SEM. Statistics are shown using ANOVA with a Tukey's post hoc test. *P<0.05, ***P<0.001.

FIGS. 7A-7D are a series of graphs showing fibronectin staining of UUO kidneys after a treatment regimen of 7-8-week-old male C57BL/6 mice with either IgG2a (n=6), ActRIIA-mFc (n=6), or ActRIIB-mFc (n=6). Data are shown as mean±SEM. Statistics are shown using ANOVA with a Tukey's post hoc test. **P<0.01.

FIGS. 8A-8D are a series of graphs showing αSMA staining of UUO kidneys after a treatment regimen of 7-8-week-old male C57BL/6 mice with either IgG2a (n=6), ActRIIA-mFc (n=6), or ActRIIB-mFc (n=6). Data are shown as mean±SEM. Statistics are shown using ANOVA with a Tukey's post hoc test. **P<0.01, ***P<0.001.

FIGS. 9A-9D are a series of graphs showing pSmad3 staining of UUO kidneys after a treatment regimen of 7-8-week-old male C57BL/6 mice with either IgG2a (n=6), ActRIIA-mFc (n=6), or ActRIIB-mFc (n=6). Data are shown as mean±SEM. Statistics are shown using ANOVA with a Tukey's post hoc test. **P<0.01, ***P<0.001.

### DETAILED DESCRIPTION OF THE INVENTION

The invention features methods of treating Alport syndrome by administering to a subject a polypeptide including an extracellular activin receptor type II (ActRII) variant, such an extracellular ActRII chimera or an extracellular ActRIIB variant. In some embodiments, a polypeptide including an extracellular ActRII variant includes an extracellular ActRII variant fused to an Fc domain monomer. A polypeptide including an extracellular ActRII variant fused to an Fc domain monomer may form a dimer (e.g., homodimer or heterodimer) through the interaction between two Fc domain monomers. The ActRII variants described herein may bind to activins (e.g., activin A and/or activin B), myostatin, and GDF-11, and may have weak binding affinity or no binding affinity to bone morphogenetic protein 9 (BMP9). Treatment of Alport syndrome with a polypeptide including an extracellular ActRII variant described herein may reduce kidney inflammation, reduce or reverse kidney fibrosis, slow or inhibit the progression of kidney fibrosis or kidney inflammation, delay the development of kidney fibrosis or kidney inflammation, slow or inhibit disease progression, improve kidney function, slow or inhibit a decline in kidney function, delay the onset of kidney failure or end stage renal disease, and/or delay or eliminate the need for dialysis or a kidney transplant.

### Extracellular Activin Receptor Type II Variants

Activin type II receptors are single transmembrane domain receptors that modulate signals for ligands in the transforming growth factor β (TGF-β) superfamily. Ligands in the TGF-β superfamily are involved in a host of physiological processes, such as muscle growth, vascular growth, cell differentiation, homeostasis, hematopoiesis, and osteogenesis. Examples of ligands in the TGF-β superfamily include, e.g., activin A, activin B, inhibin, growth differentiation factors (GDFs) (e.g., GDF8, also known as myostatin, and GDF11), and bone morphogenetic proteins (BMPs) (e.g., BMP9).

There exist two types of activin type II receptors: ActRIIA and ActRIIB. Studies have shown that BMP9 binds ActRIIB with about 300-fold higher binding affinity than ActRIIA (see, e.g., Townson et al., J. Biol. Chem. 287:27313, 2012). ActRIIA-Fc is known to have a longer half-life compared to ActRIIB-Fc. Described herein are two types of ActRII variants: 1) extracellular ActRII chimeras that are constructed by combining portions of extracellular ActRIIA and ActRIIB with the goal of generating proteins that bind to ActRII ligands (e.g., activin A, activin B, myostatin, and GDF11) and retain the function of wild-type extracellular ActRII proteins; and 2) extracellular ActRIIB variants that are constructed by introducing amino acid residues of ActRIIA into ActRIIB, or by introducing novel amino acid substitutions, with the goal of reducing BMP9 binding to prevent or reduce disruption of endogenous BMP9 signaling.

The present invention is based, in part, on the discovery that administration of a polypeptide including an ActRIIB variant described herein (an ActRIIB variant-Fc polypeptide) to human subjects led to a reduction in markers of fibrosis and inflammation. These data suggest that polypeptides including an ActRII variant, such as an ActRIIB variant, could be used to treat subjects with Alport syndrome, in which kidney fibrosis and kidney inflammation are thought to contribute to a decline in kidney function. Glomerular filtration rate (GFR), as estimated by creatinine clearance, is inversely correlated with cortical interstitial volume fraction, a measure of fibrosis of the renal interstitium. Creatinine clearance is maintained in the normal range until cortical interstitial fibrosis increases above the upper limit of the normal range, suggesting that suppression of renal interstitial fibrosis will protect kidney function in subjects with Alport syndrome. Accordingly, administration of an agent that has been shown to decrease markers of inflammation and fibrosis in human subjects, such as an ActRII variant described herein, could improve therapeutic outcomes in subjects with Alport syndrome.

### ActRII Chimeras

In some embodiments, the ActRII variant for use according to the methods described herein is an extracellular ActRII chimera constructed by combining portions of extracellular ActRIIA and ActRIIB with the goal of generating proteins that bind to ActRII ligands (e.g., activin A, activin B, myostatin, and GDF11) and retain or improve upon the function of wild-type extracellular ActRII proteins. In some embodiments, the ActRII chimeras exhibit reduced BMP9 binding relative to wild-type extracellular ActRIIB, which can prevent or reduce disruption of endogenous BMP9 signaling. In some embodiments, the chimeras have properties of both ActRIIA (e.g., low binding affinity to BMP9 and/or longer serum half-life as an Fc fusion protein) and ActRIIB (e.g., strong binding affinity to activins A and B). The ActRII chimeras may exhibit similar or improved binding to activins (e.g., activin A and/or activin B) and/or myostatin compared to wild-type extracellular ActRIIA and/or ActRIIB, allowing them to compete with endogenous activin receptors for ligand binding and reduce or inhibit endogenous activin receptor signaling. The wild-type amino acid sequences of the extracellular portions of human ActRIIA and ActRIIB are shown below.

Human ActRIIA, extracellular portion (SEQ ID NO: 44):
GAILGRSETQECLFFNANWEKDRTNQTGVEPCYGDKDKRRHCFATWKNISGSIEIVKQGCWLD
DINCYDRTDCVEKKDSPEVYFCCCEGNMCNEKFSYFPEMEVTQPTS
Human ActRIIB, extracellular portion (SEQ ID NO: 45):
GRGEAETRECIYYNANWELERTNQSGLERCEGEQDKRLHCYASWRNSSGTIELVKKGCWLDDF
NCYDRQECVATEENPQVYFCCCEGNFCNERFTHLPEAGGPEVTYEPPPTAPT

In some embodiments, the polypeptide for use according to the methods described herein is a polypeptide including an extracellular ActRII chimera that contains sequence from both the extracellular portion of ActRIIB and the extracellular portion of ActRIIA. The ActRII chimeras described herein result from joining an N-terminal portion of extracellular ActRIIB (SEQ ID NO: 45 shown above) to a C-terminal portion of extracellular ActRIIA (SEQ ID NO: 44 shown above) such that the sequences are contiguous (e.g., the ActRIIA sequence continues where the ActRIIB sequence left off, starting with the next the amino acid located in the corresponding position of ActRIIA). In some embodiments, the N-terminus of the ActRII chimera includes the six amino acids found at the N-terminus of extracellular ActRIIA joined to the fifth amino acid of extracellular ActRIIB. In some embodiments, the N-terminus of the ActRII chimera begins with the first amino acid located at the N-terminus of extracellular ActRIIB. Accordingly, in some embodiments, the N-terminal portion of ActRIIB begins with the amino acid in the fifth position of SEQ ID NO: 45 (A), while in other embodiments (e.g., in embodiments in which the six amino acids found at the N-terminus of extracellular ActRIIA are not included in the chimera), the N-terminal portion of ActRIIB begins with the amino acid in the first position of SEQ ID NO: 45 (G). In some embodiments, the N-terminus of the ActRII chimera includes the first ten amino acids found at the N-terminus of extracellular ActRIIA joined to the ninth amino acid of extracellular ActRIIB, in which case the N-terminal portion of ActRIIB begins with the amino acid in the ninth position of SEQ ID NO: 45 (E). The extracellular ActRII chimera may also include one or more amino acid substitutions in the portion of the chimera that corresponds to the sequence of ActRIIB compared to wild-type extracellular ActRIIB (e.g., SEQ ID NO: 45 shown above) and/or one or more amino acid substitutions in the portion of the chimera that corresponds to the sequence of ActRIIA compared to wild-type extracellular ActRIIA (e.g., SEQ ID NO: 44 shown above). Amino acid substitutions at 9 different positions may be introduced into an extracellular ActRII chimera (Table 1). An extracellular ActRII chimera may have one or more (e.g., 1, 2, 3, 4, 5, 6, 7, 8, or 9) amino acid substitutions relative the sequence of a wild-type sequence (e.g., relative to the sequence of wild-type extracellular ActRIIB (SEQ ID NO: 45) if the portion of the chimera corresponds to a region of wild-type extracellular ActRIIB, or relative to the sequence of wild-type extracellular ActRIIA (SEQ ID NO: 44) if the portion of the chimera corresponds to a region of wild-type extracellular ActRIIA). The positions at which amino acid substitutions may be made, as well as the amino acids that may be substituted at these positions, are listed in Table 1.

Amino acid substitutions can alter the activity and/or binding affinity of the extracellular ActRII chimeras. In some embodiments, the extracellular ActRII chimeras bind to activin A, activin B, myostatin, and/or GDF11 with sufficient affinity to compete with endogenous activin receptors for binding to one or more of these ligands. In some embodiments, the extracellular ActRII chimeras have reduced, weak, or no substantial binding to BMP9 (e.g., compared to wild-type ActRIIB). BMP9 binding may be reduced in extracellular ActRII chimeras containing the amino acid sequence TEEN (SEQ ID NO: 265) or TKEN (SEQ ID NO: 266) at positions X₃, X₄, X₅, and X₆. In some embodiments, a polypeptide including an extracellular ActRII chimera (e.g., any one of SEQ ID NOs: 1-43 (e.g., SEQ ID NOs: 22-43)) with the sequence TEEN (SEQ ID NO: 265) at positions X₃, X₄, X₅, and X₆ can have a substitution of the amino acid K for the amino acid E at position X₄. In some embodiments, a polypeptide including an extracellular ActRII chimera (e.g., any one of SEQ ID NOs: 1-43 (e.g., SEQ ID NOs: 22-43)) with the sequence TKEN (SEQ ID NO: 266) at positions X₃, X₄, X₅, and X₆ can have a substitution of the amino acid E for the amino acid K at position X₄. The sequences TEEN (SEQ ID NO: 265) and TKEN (SEQ ID NO: 266) can be used interchangeably in the extracellular ActRII chimeras (e.g., the chimeras in Tables 1 and 2, e.g., SEQ ID NOs: 1-43 (e.g., SEQ ID NOs: 22-43)). The extracellular ActRII chimeras may further include a C-terminal extension (e.g., additional amino acids at the C-terminus). The C-terminal extension can add one or more additional amino acids at the C-terminus (e.g., 1, 2, 3, 4, 5, 6 or more additional amino acids) to any of the chimeras shown in Tables 1 and 2 (e.g., SEQ ID NOs: 1-43 (e.g., SEQ ID NOs: 22-43)). The C-terminal extension may correspond to sequence from the same position in wild-type ActRIIA or ActRIIB. For example, C-terminal extensions that can be included in the extracellular ActRII chimeras of the invention are the amino acid sequence NP and the amino acid sequence NPVTPK (SEQ ID NO: 104), which correspond to sequence found in the same position in wild-type ActRIIA.

TABLE 1
Amino acid substitutions in an extracellular ActRII chimera having a sequence
of any one of SEQ ID NOs: 1-21

GAILGRAETRECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 1)
GAILGRAETRECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 2)
GAILGRAETRECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 3)
GAILGRAETRECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 4)
GAILGRAETRECIYYNANWELERTNQSGLERCEGEQX₁KRLHCYASWRNSSGTIEIVKQGCWLDDX₂X₃
CYDRTDCVX₄X₅X₆X₇X₈PX₉VYFCCCEGNMCNEKFSYFPEMEVTQPTS (SEQ ID NO: 5)
GAILGRAETRECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 6)
GAILGRAETRECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 7)
GRGEAETRECIYYNANWELERTNQSGLERCEGEQX₁KRRHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 8)
GRGEAETRECIYYNANWELERTNQSGLERCEGEQX₁KRLHCFATWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 9)
GRGEAETRECIYYNANWELERTNQSGLERCEGEQX₁KRLHCYASWKNISGSIEIVKQGCWLDDX₂X₃C
YDRTDCVX₄X₅X₆X₇X₈PX₉VYFCCCEGNMCNEKFSYFPEMEVTQPTS (SEQ ID NO: 10)
GRGEAETRECIYYNANWELERTNQSGLERCEGEQX₁KRLHCYASWRNSSGSIEIVKQGCWLDDX₂X₃C
YDRTDCVX₄X₅X₆X₇X₈PX₉VYFCCCEGNMCNEKFSYFPEMEVTQPTS (SEQ ID NO: 11)
GRGEAETRECIYYNANWELERTNQSGLERCEGEQX₁KRLHCYASWRNSSGTIEIVKQGCWLDDX₂X₃C
YDRTDCVX₄X₅X₆X₇X₈PX₉VYFCCCEGNMCNEKFSYFPEMEVTQPTS (SEQ ID NO: 12)
GRGEAETRECIYYNANWELERTNQSGLERCEGEQX₁KRLHCYASWRNSSGTIELVKKGCWLDDX₂X₃
CYDRTDCVX₄X₅X₆X₇X₈PX₉VYFCCCEGNMCNEKFSYFPEMEVTQPTS (SEQ ID NO: 13)
GRGEAETRECIYYNANWELERTNQSGLERCEGEQX₁KRLHCYASWRNSSGTIELVKKGCWLDDX₂X₃
CYDRQECVX₄X₅X₆X₇X₈PX₉VYFCCCEGNMCNEKFSYFPEMEVTQPTS (SEQ ID NO: 14)
GAILGRSETQECIYYNANWELERTNQSGLERCEGEQX₁KRRHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 15)
GAILGRSETQECIYYNANWELERTNQSGLERCEGEQX₁KRLHCFATWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 16)
GAILGRSETQECIYYNANWELERTNQSGLERCEGEQX₁KRLHCYASWKNISGSIEIVKQGCWLDDX₂X₃
CYDRTDCVX₄X₅X₆X₇X₈PX₉VYFCCCEGNMCNEKFSYFPEMEVTQPTS (SEQ ID NO: 17)
GAILGRSETQECIYYNANWELERTNQSGLERCEGEQX₁KRLHCYASWRNSSGSIEIVKQGCWLDDX₂
X₃CYDRTDCVX₄X₅X₆X₇X₈PX₉VYFCCCEGNMCNEKFSYFPEMEVTQPTS (SEQ ID NO: 18)
GAILGRSETQECIYYNANWELERTNQSGLERCEGEQX₁KRLHCYASWRNSSGTIEIVKQGCWLDDX₂
X₃CYDRTDCVX₄X₅X₆X₇X₈PX₉VYFCCCEGNMCNEKFSYFPEMEVTQPTS (SEQ ID NO: 19)
GAILGRSETQECIYYNANWELERTNQSGLERCEGEQX₁KRLHCYASWRNSSGTIELVKKGCWLDDX₂
X₃CYDRTDCVX₄X₅X₆X₇X₈PX₉VYFCCCEGNMCNEKFSYFPEMEVTQPTS (SEQ ID NO: 20)
GAILGRSETQECIYYNANWELERTNQSGLERCEGEQX₁KRLHCYASWRNSSGTIELVKKGCWLDDX₂
X₃CYDRQECVX₄X₅X₆X₇X₈PX₉VYFCCCEGNMCNEKFSYFPEMEVTQPTS (SEQ ID NO: 21)

| | | | |
|---|---|---|---|
| X₁ | D or R | X₆ | E or K |
| X₂ | I, F, E, D, Y, S, N, Q, | X₇ | E or D |
| | or T | | |
| X₃ | N or T | X₈ | N or S |
| X₄ | A or E | X₉ | Q, E, K, R, D, or N |
| X₅ | T or K | | |

In some embodiments, in the extracellular ActRII chimeras of SEQ ID NOs: 1-21 (shown in Table 1) X₁ is D, X₂ is 1, F, or E, X₃ is N or T, X₄ is A or E, X₅ is T or K, X₆ is E or K, X₇ is E or D, X₈ is N or S, and X₉ is E or Q. In some embodiments, in the extracellular ActRII chimeras of SEQ ID NOs: 1-21 X₁ is D, X₂ is I or F, X₃ is N, X₄ is A or E, X₅ is T or K, X₆ is E or K, X₇ is E or D, X₈ is N or S, and X₉ is E or Q.

In some embodiments, a polypeptide for use in the methods described herein includes an extracellular ActRII chimera having the sequence of any one of SEQ ID NOs: 22-43 (Table 2).

TABLE 2
Extracellular ActRII chimeras having the sequences of SEQ ID NOs: 22-43

| SEQ ID NO: | Amino Acid Sequence |
|---|---|
| 22 | GAILGRAETRECIYYNANWELERTNQSGLERCEGEQDKRLHCYASWRNSSGTIEIV |
| | KQGCWLDDINCYDRTDCVATEENPQVYFCCCEGNMCNEKFSYFPEMEVTQPTS |
| 23 | GRGEAETRECIYYNANWELERTNQSGLERCEGEQDKRLHCYASWRNSSGTIEIVK |
| | QGCWLDDINCYDRTDCVEKKDSPEVYFCCCEGNMCNEKFSYFPEMEVTQPTS |
| 24 | GRGEAETRECIYYNANWELERTNQSGLERCEGEQDKRLHCYASWRNSSGTIEIVK |
| | QGCWLDDINCYDRTDCVEKKDSPQVYFCCCEGNMCNEKFSYFPEMEVTQPTS |
| 25 | GRGEAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTS |
| 26 | GAILGRAETRECIYYNANWELERTNQSGLERCEGEQDKRLHCYASWRNSSGTIEIV |
| | KQGCWLDDFNCYDRTDCVATEENPQVYFCCCEGNMCNEKFSYFPEMEVTQPTS |
| 27 | GRGEAETRECIYYNANWELERTNQSGLERCEGEQDKRLHCYASWRNSSGTIEIVK |
| | QGCWLDDFNCYDRTDCVEKKDSPEVYFCCCEGNMCNEKFSYFPEMEVTQPTS |
| 28 | GRGEAETRECIYYNANWELERTNQSGLERCEGEQDKRRHCFATWKNISGSIEIVKQ |
| | GCWLDDFNCYDRTDCVEKKDSPQVYFCCCEGNMCNEKFSYFPEMEVTQPTS |
| 29 | GRGEAETRECIYYNANWELERTNQSGLERCEGEQDKRLHCFATWKNISGSIEIVKQ |
| | GCWLDDFNCYDRTDCVEKKDSPQVYFCCCEGNMCNEKFSYFPEMEVTQPTS |
| 30 | GRGEAETRECIYYNANWELERTNQSGLERCEGEQDKRLHCYASWKNISGSIEIVKQ |
| | GCWLDDFNCYDRTDCVEKKDSPQVYFCCCEGNMCNEKFSYFPEMEVTQPTS |
| 31 | GRGEAETRECIYYNANWELERTNQSGLERCEGEQDKRLHCYASWRNSSGSIEIVK |
| | QGCWLDDFNCYDRTDCVEKKDSPQVYFCCCEGNMCNEKFSYFPEMEVTQPTS |
| 32 | GRGEAETRECIYYNANWELERTNQSGLERCEGEQDKRLHCYASWRNSSGTIELVK |
| | KGCWLDDFNCYDRTDCVEKKDSPQVYFCCCEGNMCNEKFSYFPEMEVTQPTS |
| 33 | GRGEAETRECIYYNANWELERTNQSGLERCEGEQDKRLHCYASWRNSSGTIELVK |
| | KGCWLDDFNCYDRQECVATKDSPQVYFCCCEGNMCNEKFSYFPEMEVTQPTS |
| 34 | GRGEAETRECIYYNANWELERTNQSGLERCEGEQDKRLHCYASWRNSSGTIELVK |
| | KGCWLDDFNCYDRQECVATEENPQVYFCCCEGNMCNEKFSYFPEMEVTQPTS |
| 35 | GRGEAETRECIYYNANWELERTNQSGLERCEGEQDKRLHCYASWRNSSGTIEIVK |
| | QGCWLDDNNCYDRTDCVEKKDSPQVYFCCCEGNMCNEKFSYFPEMEVTQPTS |
| 36 | GRGEAETRECIYYNANWELERTNQSGLERCEGEQDKRLHCYASWRNSSGTIEIVK |
| | QGCWLDDTNCYDRTDCVEKKDSPQVYFCCCEGNMCNEKFSYFPEMEVTQPTS |
| 37 | GRGEAETRECIYYNANWELERTNQSGLERCEGEQDKRLHCYASWRNSSGTIEIVK |
| | QGCWLDDETCYDRTDCVEKKDSPQVYFCCCEGNMCNEKFSYFPEMEVTQPTS |
| 38 | GRGEAETRECIYYNANWELERTNQSGLERCEGEQRKRLHCYASWRNSSGTIEIVK |
| | QGCWLDDFNCYDRTDCVEKKDSPQVYFCCCEGNMCNEKFSYFPEMEVTQPTS |
| 39 | GRGEAETRECIYYNANWELERTNQSGLERCEGEQDKRLHCYASWRNSSGTIEIVK |
| | QGCWLDDFNCYDRTDCVETKDSPQVYFCCCEGNMCNEKFSYFPEMEVTQPTS |
| 40 | GAILGRAETRECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 41 | GAILGRSETQECIYYNANWELERTNQSGLERCEGEQDKRLHCYASWRNSSGTIEIV |
| | KQGCWLDDFNCYDRTDCVEKKDSPQVYFCCCEGNMCNEKFSYFPEMEVTQPTS |
| 42 | GRGEAETRECIYYNANWELERTNQSGLERCEGEQDKRLHCYASWRNSSGTIELVK |
| | KGCWLDDFNCYDRQECVATKDSPEVYFCCCEGNMCNEKFSYFPEMEVTQPTS |
| 43 | GRGEAETRECIYYNANWELERTNQSGLERCEGEQDKRLHCYASWRNSSGTIELVK |
| | KGCWLDDFNCYDRQECVATKENPQVYFCCCEGNMCNEKFSYFPEMEVTQPTS |

In some embodiments, the extracellular ActRII chimeras described herein have an N-terminal truncation of 1-9 amino acids (e.g., 1, 2, 3, 4, 5, 6, 7, 8, or 9 amino acids). The N-terminal truncation can involve the removal of 1-9 amino acids from the N-terminus of any of the chimeras shown in Tables 1 and 2 (e.g., SEQ ID NOs: 1-43 (e.g., SEQ ID NOs: 22-43)). The N-terminal truncation can remove amino acids up to two amino acids before the first cysteine (e.g., the two amino acids before the first cysteine (RE or QE) are retained in the N-terminally truncated ActRII chimeras). Exemplary ActRII chimeras having N-terminal truncations are provided in Table 3, below.

TABLE 3
Extracellular ActRII chimeras having N-terminal truncations

| SEQ ID NO: | Amino Acid Sequence |
|---|---|
| 111 | ILGRAETRECIYYNANWELERTNQSGLERCEGEQDKRLHCYASWRNSSGTIEIVKQ |
| | GCWLDDINCYDRTDCVATEENPQVYFCCCEGNMCNEKFSYFPEMEVTQPTS |
| 112 | RGEAETRECIYYNANWELERTNQSGLERCEGEQDKRLHCYASWRNSSGTIEIVKQ |
| | GCWLDDINCYDRTDCVEKKDSPEVYFCCCEGNMCNEKFSYFPEMEVTQPTS |
| 113 | RGEAETRECIYYNANWELERTNQSGLERCEGEQDKRLHCYASWRNSSGTIEIVKQ |
| | GCWLDDINCYDRTDCVEKKDSPQVYFCCCEGNMCNEKFSYFPEMEVTQPTS |
| 114 | RGEAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 115 | LGRAETRECIYYNANWELERTNQSGLERCEGEQDKRLHCYASWRNSSGTIEIVKQ |
| | GCWLDDINCYDRTDCVATEENPQVYFCCCEGNMCNEKFSYFPEMEVTQPTS |
| 116 | GEAETRECIYYNANWELERTNQSGLERCEGEQDKRLHCYASWRNSSGTIEIVKQG |
| | CWLDDINCYDRTDCVEKKDSPEVYFCCCEGNMCNEKFSYFPEMEVTQPTS |
| 117 | GEAETRECIYYNANWELERTNQSGLERCEGEQDKRLHCYASWRNSSGTIEIVKQG |
| | CWLDDINCYDRTDCVEKKDSPQVYFCCCEGNMCNEKFSYFPEMEVTQPTS |
| 118 | GEAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 119 | GRAETRECIYYNANWELERTNQSGLERCEGEQDKRLHCYASWRNSSGTIEIVKQG |
| | CWLDDINCYDRTDCVATEENPQVYFCCCEGNMCNEKFSYFPEMEVTQPTS |
| 120 | EAETRECIYYNANWELERTNQSGLERCEGEQDKRLHCYASWRNSSGTIEIVKQGC |
| | WLDDINCYDRTDCVEKKDSPEVYFCCCEGNMCNEKFSYFPEMEVTQPTS |
| 121 | EAETRECIYYNANWELERTNQSGLERCEGEQDKRLHCYASWRNSSGTIEIVKQGC |
| | WLDDINCYDRTDCVEKKDSPQVYFCCCEGNMCNEKFSYFPEMEVTQPTS |
| 122 | EAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 123 | RAETRECIYYNANWELERTNQSGLERCEGEQDKRLHCYASWRNSSGTIEIVKQGC |
| | WLDDINCYDRTDCVATEENPQVYFCCCEGNMCNEKFSYFPEMEVTQPTS |
| 124 | AETRECIYYNANWELERTNQSGLERCEGEQDKRLHCYASWRNSSGTIEIVKQGCW |
| | LDDINCYDRTDCVEKKDSPEVYFCCCEGNMCNEKFSYFPEMEVTQPTS |
| 125 | AETRECIYYNANWELERTNQSGLERCEGEQDKRLHCYASWRNSSGTIEIVKQGCW |
| | LDDINCYDRTDCVEKKDSPQVYFCCCEGNMCNEKFSYFPEMEVTQPTS |
| 126 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 127 | AETRECIYYNANWELERTNQSGLERCEGEQDKRLHCYASWRNSSGTIEIVKQGCW |
| | LDDINCYDRTDCVATEENPQVYFCCCEGNMCNEKFSYFPEMEVTQPTS |
| 128 | ETRECIYYNANWELERTNQSGLERCEGEQDKRLHCYASWRNSSGTIEIVKQGCWL |
| | DDINCYDRTDCVEKKDSPEVYFCCCEGNMCNEKFSYFPEMEVTQPTS |
| 129 | ETRECIYYNANWELERTNQSGLERCEGEQDKRLHCYASWRNSSGTIEIVKQGCWL |
| | DDINCYDRTDCVEKKDSPQVYFCCCEGNMCNEKFSYFPEMEVTQPTS |
| 130 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 131 | ETRECIYYNANWELERTNQSGLERCEGEQDKRLHCYASWRNSSGTIEIVKQGCWL |
| | DDINCYDRTDCVATEENPQVYFCCCEGNMCNEKFSYFPEMEVTQPTS |
| 132 | TRECIYYNANWELERTNQSGLERCEGEQDKRLHCYASWRNSSGTIEIVKQGCWLD |
| | DINCYDRTDCVEKKDSPEVYFCCCEGNMCNEKFSYFPEMEVTQPTS |
| 133 | TRECIYYNANWELERTNQSGLERCEGEQDKRLHCYASWRNSSGTIEIVKQGCWLD |
| | DINCYDRTDCVEKKDSPQVYFCCCEGNMCNEKFSYFPEMEVTQPTS |
| 134 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 135 | TRECIYYNANWELERTNQSGLERCEGEQDKRLHCYASWRNSSGTIEIVKQGCWLD |
| | DINCYDRTDCVATEENPQVYFCCCEGNMCNEKFSYFPEMEVTQPTS |
| 136 | RECIYYNANWELERTNQSGLERCEGEQDKRLHCYASWRNSSGTIEIVKQGCWLDD |
| | INCYDRTDCVEKKDSPEVYFCCCEGNMCNEKFSYFPEMEVTQPTS |
| 137 | RECIYYNANWELERTNQSGLERCEGEQDKRLHCYASWRNSSGTIEIVKQGCWLDD |
| | INCYDRTDCVEKKDSPQVYFCCCEGNMCNEKFSYFPEMEVTQPTS |
| 138 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |
| 139 | RECIYYNANWELERTNQSGLERCEGEQDKRLHCYASWRNSSGTIEIVKQGCWLDD |
| | INCYDRTDCVATEENPQVYFCCCEGNMCNEKFSYFPEMEVTQPTS |
| 140 | RGEAETRECIYYNANWELERTNQSGLERCEGEQDKRRHCFATWKNISGSIEIVKQG |
| | CWLDDFNCYDRTDCVEKKDSPQVYFCCCEGNMCNEKFSYFPEMEVTQPTS |
| 141 | RGEAETRECIYYNANWELERTNQSGLERCEGEQDKRLHCYASWRNSSGTIEIVKQ |
| | GCWLDDETCYDRTDCVEKKDSPQVYFCCCEGNMCNEKFSYFPEMEVTQPTS |
| 142 | ILGRAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 143 | ILGRSETQECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 144 | RGEAETRECIYYNANWELERTNQSGLERCEGEQDKRLHCYASWRNSSGTIELVKK |
| | GCWLDDFNCYDRQECVATKDSPEVYFCCCEGNMCNEKFSYFPEMEVTQPTS |
| 145 | RGEAETRECIYYNANWELERTNQSGLERCEGEQDKRLHCYASWRNSSGTIELVKK |
| | GCWLDDFNCYDRQECVATKENPQVYFCCCEGNMCNEKFSYFPEMEVTQPTS |
| 146 | GEAETRECIYYNANWELERTNQSGLERCEGEQDKRRHCFATWKNISGSIEIVKQGC |
| | WLDDFNCYDRTDCVEKKDSPQVYFCCCEGNMCNEKFSYFPEMEVTQPTS |
| 147 | GEAETRECIYYNANWELERTNQSGLERCEGEQDKRLHCYASWRNSSGTIEIVKQG |
| | CWLDDETCYDRTDCVEKKDSPQVYFCCCEGNMCNEKFSYFPEMEVTQPTS |
| 148 | LGRAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 149 | LGRSETQECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTS |
| 150 | GEAETRECIYYNANWELERTNQSGLERCEGEQDKRLHCYASWRNSSGTIELVKKG |
| | CWLDDFNCYDRQECVATKDSPEVYFCCCEGNMCNEKFSYFPEMEVTQPTS |
| 151 | GEAETRECIYYNANWELERTNQSGLERCEGEQDKRLHCYASWRNSSGTIELVKKG |
| | CWLDDFNCYDRQECVATKENPQVYFCCCEGNMCNEKFSYFPEMEVTQPTS |
| 152 | EAETRECIYYNANWELERTNQSGLERCEGEQDKRRHCFATWKNISGSIEIVKQGC |
| | WLDDFNCYDRTDCVEKKDSPQVYFCCCEGNMCNEKFSYFPEMEVTQPTS |
| 153 | EAETRECIYYNANWELERTNQSGLERCEGEQDKRLHCYASWRNSSGTIEIVKQGC |
| | WLDDETCYDRTDCVEKKDSPQVYFCCCEGNMCNEKFSYFPEMEVTQPTS |
| 154 | GRAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 155 | GRSETQECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTS |
| 156 | EAETRECIYYNANWELERTNQSGLERCEGEQDKRLHCYASWRNSSGTIELVKKGC |
| | WLDDFNCYDRQECVATKDSPEVYFCCCEGNMCNEKFSYFPEMEVTQPTS |
| 157 | EAETRECIYYNANWELERTNQSGLERCEGEQDKRLHCYASWRNSSGTIELVKKGC |
| | WLDDFNCYDRQECVATKENPQVYFCCCEGNMCNEKFSYFPEMEVTQPTS |
| 158 | AETRECIYYNANWELERTNQSGLERCEGEQDKRRHCFATWKNISGSIEIVKQGCW |
| | LDDFNCYDRTDCVEKKDSPQVYFCCCEGNMCNEKFSYFPEMEVTQPTS |
| 159 | AETRECIYYNANWELERTNQSGLERCEGEQDKRLHCYASWRNSSGTIEIVKQGCW |
| | LDDETCYDRTDCVEKKDSPQVYFCCCEGNMCNEKFSYFPEMEVTQPTS |
| 160 | RAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 161 | RSETQECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTS |
| 162 | AETRECIYYNANWELERTNQSGLERCEGEQDKRLHCYASWRNSSGTIELVKKGCW |
| | LDDFNCYDRQECVATKDSPEVYFCCCEGNMCNEKFSYFPEMEVTQPTS |
| 163 | AETRECIYYNANWELERTNQSGLERCEGEQDKRLHCYASWRNSSGTIELVKKGCW |
| | LDDFNCYDRQECVATKENPQVYFCCCEGNMCNEKFSYFPEMEVTQPTS |
| 164 | ETRECIYYNANWELERTNQSGLERCEGEQDKRRHCFATWKNISGSIEIVKQGCWL |
| | DDFNCYDRTDCVEKKDSPQVYFCCCEGNMCNEKFSYFPEMEVTQPTS |
| 165 | ETRECIYYNANWELERTNQSGLERCEGEQDKRLHCYASWRNSSGTIEIVKQGCWL |
| | DDETCYDRTDCVEKKDSPQVYFCCCEGNMCNEKFSYFPEMEVTQPTS |
| 166 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 167 | SETQECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTS |
| 168 | ETRECIYYNANWELERTNQSGLERCEGEQDKRLHCYASWRNSSGTIELVKKGCWL |
| | DDFNCYDRQECVATKDSPEVYFCCCEGNMCNEKFSYFPEMEVTQPTS |
| 169 | ETRECIYYNANWELERTNQSGLERCEGEQDKRLHCYASWRNSSGTIELVKKGCWL |
| | DDFNCYDRQECVATKENPQVYFCCCEGNMCNEKFSYFPEMEVTQPTS |
| 170 | TRECIYYNANWELERTNQSGLERCEGEQDKRRHCFATWKNISGSIEIVKQGCWLD |
| | DFNCYDRTDCVEKKDSPQVYFCCCEGNMCNEKFSYFPEMEVTQPTS |
| 171 | TRECIYYNANWELERTNQSGLERCEGEQDKRLHCYASWRNSSGTIEIVKQGCWLD |
| | DETCYDRTDCVEKKDSPQVYFCCCEGNMCNEKFSYFPEMEVTQPTS |
| 172 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 173 | ETQECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTS |
| 174 | TRECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTS |
| 175 | TRECIYYNANWELERTNQSGLERCEGEQDKRLHCYASWRNSSGTIELVKKGCWLD |
| | DENCYDRQECVATKENPQVYFCCCEGNMCNEKFSYFPEMEVTQPTS |
| 176 | RECIYYNANWELERTNQSGLERCEGEQDKRRHCFATWKNISGSIEIVKQGCWLDD |
| | FNCYDRTDCVEKKDSPQVYFCCCEGNMCNEKFSYFPEMEVTQPTS |
| 177 | RECIYYNANWELERTNQSGLERCEGEQDKRLHCYASWRNSSGTIEIVKQGCWLDD |
| | ETCYDRTDCVEKKDSPQVYFCCCEGNMCNEKFSYFPEMEVTQPTS |
| 178 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 179 | TQECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTS |
| 180 | RECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTS |
| 181 | RECIYYNANWELERTNQSGLERCEGEQDKRLHCYASWRNSSGTIELVKKGCWLD |
| | DFNCYDRQECVATKENPQVYFCCCEGNMCNEKFSYFPEMEVTQPTS |
| 182 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |
| 183 | QECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTS |

In some embodiments, a polypeptide including an extracellular ActRII chimera may further include an Fc domain monomer, which may be fused to the N- or C-terminus (e.g., C-terminus) of the extracellular ActRII chimera by way of a linker or other covalent bonds. A polypeptide including an extracellular ActRII chimera fused to an Fc domain monomer may form a dimer (e.g., homodimer or heterodimer) through the interaction between two Fc domain monomers, which combine to form an Fc domain in the dimer. Exemplary polypeptides containing an ActRII chimera, an Fc domain monomer, and a linker are provided in Table 4, below. In some embodiments, the terminal lysine is absent from the Fc domain monomer amino acid sequence included in the polypeptides of Table 4.

TABLE 4
Polypeptides containing an extracellular ActRII chimera fused
to an Fc domain monomer by way of a linker

| SEQ ID NO: | Amino Acid Sequence |
|---|---|
| 107 | GAILGRAETRECIYYNANWELERTNQSGLERCEGEQDKRLHCYASWRNSSGTIEI |
| | VKQGCWLDDINCYDRTDCVATEENPQVYFCCCEGNMCNEKFSYFPEMEVTQPTSG |
| | GGDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQ |
| | KSLSLSPGK |
| 108 | GRGEAETRECIYYNANWELERTNQSGLERCEGEQDKRLHCYASWRNSSGTIEIVK |
| | QGCWLDDINCYDRTDCVEKKDSPEVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 109 | GRGEAETRECIYYNANWELERTNQSGLERCEGEQDKRLHCYASWRNSSGTIEIVK |
| | QGCWLDDINCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 110 | GRGEAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 184 | ILGRAETRECIYYNANWELERTNQSGLERCEGEQDKRLHCYASWRNSSGTIEIVK |
| | QGCWLDDINCYDRTDCVATEENPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 185 | RGEAETRECIYYNANWELERTNQSGLERCEGEQDKRLHCYASWRNSSGTIEIVKQ |
| | GCWLDDINCYDRTDCVEKKDSPEVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 186 | RGEAETRECIYYNANWELERTNQSGLERCEGEQDKRLHCYASWRNSSGTIEIVKQ |
| | GCWLDDINCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 187 | RGEAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 188 | LGRAETRECIYYNANWELERTNQSGLERCEGEQDKRLHCYASWRNSSGTIEIVKQ |
| | GCWLDDINCYDRTDCVATEENPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 189 | GEAETRECIYYNANWELERTNQSGLERCEGEQDKRLHCYASWRNSSGTIEIVKQG |
| | CWLDDINCYDRTDCVEKKDSPEVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 190 | GEAETRECIYYNANWELERTNQSGLERCEGEQDKRLHCYASWRNSSGTIEIVKQG |
| | CWLDDINCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 191 | GEAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 192 | GRAETRECIYYNANWELERTNQSGLERCEGEQDKRLHCYASWRNSSGTIEIVKQG |
| | CWLDDINCYDRTDCVATEENPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 193 | EAETRECIYYNANWELERTNQSGLERCEGEQDKRLHCYASWRNSSGTIEIVKQGC |
| | WLDDINCYDRTDCVEKKDSPEVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 194 | EAETRECIYYNANWELERTNQSGLERCEGEQDKRLHCYASWRNSSGTIEIVKQGC |
| | WLDDINCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 195 | EAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 196 | RAETRECIYYNANWELERTNQSGLERCEGEQDKRLHCYASWRNSSGTIEIVKQGC |
| | WLDDINCYDRTDCVATEENPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 197 | AETRECIYYNANWELERTNQSGLERCEGEQDKRLHCYASWRNSSGTIEIVKQGCW |
| | LDDINCYDRTDCVEKKDSPEVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 198 | AETRECIYYNANWELERTNQSGLERCEGEQDKRLHCYASWRNSSGTIEIVKQGCW |
| | LDDINCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 199 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 200 | AETRECIYYNANWELERTNQSGLERCEGEQDKRLHCYASWRNSSGTIEIVKQGCW |
| | LDDINCYDRTDCVATEENPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 201 | ETRECIYYNANWELERTNQSGLERCEGEQDKRLHCYASWRNSSGTIEIVKQGCWL |
| | DDINCYDRTDCVEKKDSPEVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 202 | ETRECIYYNANWELERTNQSGLERCEGEQDKRLHCYASWRNSSGTIEIVKQGCWL |
| | DDINCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 203 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 204 | ETRECIYYNANWELERTNQSGLERCEGEQDKRLHCYASWRNSSGTIEIVKQGCWL |
| | DDINCYDRTDCVATEENPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 205 | TRECIYYNANWELERTNQSGLERCEGEQDKRLHCYASWRNSSGTIEIVKQGCWLD |
| | DINCYDRTDCVEKKDSPEVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 206 | TRECIYYNANWELERTNQSGLERCEGEQDKRLHCYASWRNSSGTIEIVKQGCWLD |
| | DINCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 207 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 208 | TRECIYYNANWELERTNQSGLERCEGEQDKRLHCYASWRNSSGTIEIVKQGCWLD |
| | DINCYDRTDCVATEENPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 209 | RECIYYNANWELERTNQSGLERCEGEQDKRLHCYASWRNSSGTIEIVKQGCWLDD |
| | INCYDRTDCVEKKDSPEVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 210 | RECIYYNANWELERTNQSGLERCEGEQDKRLHCYASWRNSSGTIEIVKQGCWLDD |
| | INCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 211 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 212 | RECIYYNANWELERTNQSGLERCEGEQDKRLHCYASWRNSSGTIEIVKQGCWLDD |
| | INCYDRTDCVATEENPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 213 | GRGEAETRECIYYNANWELERTNQSGLERCEGEQDKRRHCFATWKNISGSIEIVK |
| | QGCWLDDFNCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 214 | GRGEAETRECIYYNANWELERTNQSGLERCEGEQDKRLHCYASWRNSSGTIEIVK |
| | QGCWLDDETCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 215 | GAILGRAETRECIYYNANWELERTNQSGLERCEGEQDKRLHCYASWRNSSGTIEI |
| | VKQGCWLDDFNCYDRTDCVEKKDSPQVYFCCCEGNMCNEKFSYFPEMEVTQPTSG |
| | GGDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQ |
| | KSLSLSPGK |
| 216 | GAILGRSETQECIYYNANWELERTNQSGLERCEGEQDKRLHCYASWRNSSGTIEI |
| | VKQGCWLDDFNCYDRTDCVEKKDSPQVYFCCCEGNMCNEKFSYFPEMEVTQPTSG |
| | GGDKTHTCPPCPAPELLGGPSVFLFPPKPKDTLMISRTPEVTCVVVDVSHEDPEV |
| | KFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQ |
| | KSLSLSPGK |
| 217 | GRGEAETRECIYYNANWELERTNQSGLERCEGEQDKRLHCYASWRNSSGTIELVK |
| | KGCWLDDFNCYDRQECVATKDSPEVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 218 | GRGEAETRECIYYNANWELERTNQSGLERCEGEQDKRLHCYASWRNSSGTIELVK |
| | KGCWLDDFNCYDRQECVATKENPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 219 | RGEAETRECIYYNANWELERTNQSGLERCEGEQDKRRHCFATWKNISGSIEIVKQ |
| | GCWLDDFNCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKEN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 220 | RGEAETRECIYYNANWELERTNQSGLERCEGEQDKRLHCYASWRNSSGTIEIVKQ |
| | GCWLDDETCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 221 | ILGRAETRECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 222 | ILGRSETQECIYYNANWELERTNQSGLERCEGEQDKRLHCYASWRNSSGTIEIVK |
| | QGCWLDDFNCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |
| 223 | RGEAETRECIYYNANWELERTNQSGLERCEGEQDKRLHCYASWRNSSGTIELVKK |
| | GCWLDDFNCYDRQECVATKDSPEVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 224 | RGEAETRECIYYNANWELERTNQSGLERCEGEQDKRLHCYASWRNSSGTIELVKK |
| | GCWLDDFNCYDRQECVATKENPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 225 | GEAETRECIYYNANWELERTNQSGLERCEGEQDKRRHCFATWKNISGSIEIVKQG |
| | CWLDDFNCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 226 | GEAETRECIYYNANWELERTNQSGLERCEGEQDKRLHCYASWRNSSGTIEIVKQG |
| | CWLDDETCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 227 | LGRAETRECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 228 | LGRSETQECIYYNANWELERTNQSGLERCEGEQDKRLHCYASWRNSSGTIEIVKQ |
| | GCWLDDFNCYDRTDCVEKKDSPQVYFCCCEGNMCNEKFSYFPEMEVTQPTSGGGD |
| | KTHTCPPCPAPELLGGPSVFLFPPKPKDTLMISRTPEVTCVVVDVSHEDPEVKFN |
| | WYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSL |
| | SLSPGK |
| 229 | GEAETRECIYYNANWELERTNQSGLERCEGEQDKRLHCYASWRNSSGTIELVKKG |
| | CWLDDFNCYDRQECVATKDSPEVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 230 | GEAETRECIYYNANWELERTNQSGLERCEGEQDKRLHCYASWRNSSGTIELVKKG |
| | CWLDDFNCYDRQECVATKENPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 231 | EAETRECIYYNANWELERTNQSGLERCEGEQDKRRHCFATWKNISGSIEIVKQGC |
| | WLDDFNCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 232 | EAETRECIYYNANWELERTNQSGLERCEGEQDKRLHCYASWRNSSGTIEIVKQGC |
| | WLDDETCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 233 | GRAETRECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 234 | GRSETQECIYYNANWELERTNQSGLERCEGEQDKRLHCYASWRNSSGTIEIVKQG |
| | CWLDDFNCYDRTDCVEKKDSPQVYFCCCEGNMCNEKFSYFPEMEVTQPTSGGGDK |
| | THTCPPCPAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNKALPAPI |
| | EKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLS |
| | LSPGK |
| 235 | EAETRECIYYNANWELERTNQSGLERCEGEQDKRLHCYASWRNSSGTIELVKKGC |
| | WLDDFNCYDRQECVATKDSPEVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 236 | EAETRECIYYNANWELERTNQSGLERCEGEQDKRLHCYASWRNSSGTIELVKKGC |
| | WLDDFNCYDRQECVATKENPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 237 | AETRECIYYNANWELERTNQSGLERCEGEQDKRRHCFATWKNISGSIEIVKQGCW |
| | LDDFNCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 238 | AETRECIYYNANWELERTNQSGLERCEGEQDKRLHCYASWRNSSGTIEIVKQGCW |
| | LDDETCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 239 | RAETRECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 240 | RSETQECIYYNANWELERTNQSGLERCEGEQDKRLHCYASWRNSSGTIEIVKQGC |
| | WLDDFNCYDRTDCVEKKDSPQVYFCCCEGNMCNEKFSYFPEMEVTQPTSGGGDKT |
| | HTCPPCPAPELLGGPSVFLFPPKPKDTLMISRTPEVTCVVVDVSHEDPEVKFNWY |
| | VDGVEVHNAKTKPREEQYNSTYRVVSVLTVLHQDWLNGKEYKCKVSNKALPAPIE |
| | KTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSL |
| | SPGK |
| 241 | AETRECIYYNANWELERTNQSGLERCEGEQDKRLHCYASWRNSSGTIELVKKGCW |
| | LDDFNCYDRQECVATKDSPEVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 242 | AETRECIYYNANWELERTNQSGLERCEGEQDKRLHCYASWRNSSGTIELVKKGCW |
| | LDDFNCYDRQECVATKENPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 243 | ETRECIYYNANWELERTNQSGLERCEGEQDKRRHCFATWKNISGSIEIVKQGCWL |
| | DDFNCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 244 | ETRECIYYNANWELERTNQSGLERCEGEQDKRLHCYASWRNSSGTIEIVKQGCWL |
| | DDETCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 245 | AETRECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 246 | SETQECIYYNANWELERTNQSGLERCEGEQDKRLHCYASWRNSSGTIEIVKQGCW |
| | LDDFNCYDRTDCVEKKDSPQVYFCCCEGNMCNEKFSYFPEMEVTQPTSGGGDKTH |
| | TCPPCPAPELLGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALPAPIEK |
| | TISKAKGQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQKSLSLS |
| | PGK |
| 247 | ETRECIYYNANWELERTNQSGLERCEGEQDKRLHCYASWRNSSGTIELVKKGCWL |
| | DDFNCYDRQECVATKDSPEVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 248 | ETRECIYYNANWELERTNQSGLERCEGEQDKRLHCYASWRNSSGTIELVKKGCWL |
| | DDFNCYDRQECVATKENPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 249 | TRECIYYNANWELERTNQSGLERCEGEQDKRRHCFATWKNISGSIEIVKQGCWLD |
| | DFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 250 | TRECIYYNANWELERTNQSGLERCEGEQDKRLHCYASWRNSSGTIEIVKQGCWLD |
| | DETCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 251 | QECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 252 | ETRECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 253 | ETQECIYYNANWELERTNQSGLERCEGEQDKRLHCYASWRNSSGTIEIVKQGCWL |
| | DDFNCYDRTDCVEKKDSPQVYFCCCEGNMCNEKFSYFPEMEVTQPTSGGGDKTHT |
| | CPPCPAPELLGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALPAPIEKT |
| | ISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSP |
| | GK |
| 254 | TRECIYYNANWELERTNQSGLERCEGEQDKRLHCYASWRNSSGTIELVKKGCWLD |
| | DFNCYDRQECVATKDSPEVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 255 | TRECIYYNANWELERTNQSGLERCEGEQDKRLHCYASWRNSSGTIELVKKGCWLD |
| | DFNCYDRQECVATKENPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 256 | RECIYYNANWELERTNQSGLERCEGEQDKRRHCFATWKNISGSIEIVKQGCWLDD |
| | FNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 257 | RECIYYNANWELERTNQSGLERCEGEQDKRLHCYASWRNSSGTIEIVKQGCWLDD |
| | ETCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 258 | TRECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 259 | TQECIYYNANWELERTNQSGLERCEGEQDKRLHCYASWRNSSGTIEIVKQGCWLD |
| | DFNCYDRTDCVEKKDSPQVYFCCCEGNMCNEKFSYFPEMEVTQPTSGGGDKTHTC |
| | PPCPAPELLGGPSVFLFPPKPKDTLMISRTPEVTCVVVDVSHEDPEVKFNWYVDG |
| | VEVHNAKTKPREEQYNSTYRVVSVLTVLHQDWLNGKEYKCKVSNKALPAPIEKTI |
| | SKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPG |
| | K |
| 260 | RECIYYNANWELERTNQSGLERCEGEQDKRLHCYASWRNSSGTIELVKKGCWLDD |
| | FNCYDRQECVATKDSPEVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 261 | RECIYYNANWELERTNQSGLERCEGEQDKRLHCYASWRNSSGTIELVKKGCWLDD |
| | FNCYDRQECVATKENPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 262 | RECIYYNANWELERTNQSGLERCEGEQDKRLHCYASWRNSSGTIEIVKQGCWLDD |
| | FNCYDRTDCVEKKDSPQVYFCCCEGNMCNEKFSYFPEMEVTQPTSGGGDKTHTCP |
| | PCPAPELLGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALPAPIEKTIS |
| | KAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK |
| 263 | GRGEAETRECIYYNANWELERTNQSGLERCEGEQRKRLHCYASWRNSSGTIEIVK |
| | QGCWLDDFNCYDRTDCVEKKDSPQVYFCCCEGNMCNEKFSYFPEMEVTQPTSGGG |
| | DKTHTCPPCPAPELLGGPSVFLFPPKPKDTLMISRTPEVTCVVVDVSHEDPEVKF |
| | NWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKS |
| | LSLSPGK |

Furthermore, in some embodiments, a polypeptide described herein (e.g., an ActRII chimera-Fc fusion protein) has a serum half-life of at least 7 days in humans. The polypeptide may bind to activin A with a K_{D} of 10 pM or higher. In some embodiments, the polypeptide binds to activin A, activin B, and/or myostatin and exhibits reduced (e.g., weak) binding to BMP9 (e.g., compared to wild-type extracellular ActRIIB). In some embodiments, the polypeptide that has reduced or weak binding to BMP9 has the sequence TEEN (SEQ ID NO: 265) or TKEN (SEQ ID NO: 266) at positions X₃, X₄, X₅, and X₆. In some embodiments, the polypeptide that has reduced or weak binding to BMP9 has the sequence KKDS (SEQ ID NO: 267) or TKDS (SEQ ID NO: 268) at positions X₃, X₄, X₅, and X₆. In some embodiments, the polypeptide does not substantially bind to human BMP9.

In some embodiments, the polypeptide including an extracellular ActRII chimera may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 30 pM). In some embodiments, the polypeptide may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 5 pM). The polypeptide may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

### ActRIIB Variants

In some embodiments, the ActRII variant for use according to the methods described herein is an extracellular ActRIIB variant constructed by introducing amino acid residues of ActRIIA into ActRIIB, or by introducing novel amino acid substitutions into ActRIIB, with the goal of reducing BMP9 binding to prevent or reduce disruption of endogenous BMP9 signaling. The amino acid substitutions may also impart beneficial physiological and pharmacokinetic properties of ActRIIA, such as longer half-life as an Fc fusion protein. The preferred ActRIIB variants also exhibit similar or improved binding to activins and/or myostatin compared to wild-type ActRIIB, which allows them to compete with endogenous activin receptors for ligand binding and reduce or inhibit endogenous activin receptor signaling. In some embodiments, amino acid substitutions may be introduced into an extracellular ActRIIB variant to reduce or remove the binding affinity of the variant to BMP9.

Polypeptides described herein include an extracellular ActRIIB variant having at least one amino acid substitution relative to the wild-type extracellular ActRIIB having the sequence of SEQ ID NO: 45. Possible amino acid substitutions at 28 different positions may be introduced to an extracellular ActRIIB variant (Table 5). An extracellular ActRIIB variant may have one or more (e.g., 1-28, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, 27, or 28) amino acid substitutions relative the sequence of a wild-type extracellular ActRIIB (SEQ ID NO: 45). In some embodiments, an extracellular ActRIIB variant (e.g., an extracellular ActRIIB variant having a sequence of SEQ ID NO: 269) may include amino acid substitutions at all of the 28 positions as listed in Table 5. In some embodiments, an extracellular ActRIIB variant may include amino acid substitutions at a number of positions, e.g., at 3, 4, 5, 6, 7, 8, 9, 10, 12, 15, 16, 18, 20, 22, 24, 26, or 27 out of the 28 positions, as listed in Table 5. In some embodiments, the substitutions are substitutions of an amino acid from ActRIIA into the same position in ActRIIB. In some embodiments, the substitutions are novel changes (e.g., substitutions of amino acids that are not in the corresponding position of ActRIIA, e.g., S48T, I51L, Q69D, or E70T).

Amino acid substitutions can worsen or improve the activity and/or binding affinity of the ActRIIB variants of the invention (e.g., an extracellular ActRIIB variant having the sequence of any one of SEQ ID NOs: 269-283 (e.g., SEQ ID NOs: 270-283)). In some embodiments, the amino acid substitutions worsen the binding affinity of the ActRIIB variants to BMP9 (e.g., the variants have reduced binding to BMP9 relative to wild-type extracellular ActRIIB or have lower binding to BMP9 than to other ActRIIB ligands (e.g., activin A or B, myostatin, or GDF-11)). In some embodiments, the ActRIIB variants have reduced, weak, or no substantial binding to BMP9. In some embodiments, the amino acid substitutions improve the binding affinity of ActRIIB to myostatin, activin A or B, and/or GDF-11 (e.g., the variants have improved binding affinity relative to wild-type extracellular ActRIIB or bind more strongly to myostatin, activin A or B, or GDF-11 than to BMP9). In some embodiments, the amino acid substitutions reduce the binding affinity of ActRIIB to myostatin, activin A or B, and/or GDF-11 (e.g., the variants have decreased binding affinity relative to wild-type extracellular ActRIIB or bind more weakly to myostatin, activin A or B, or GDF-11 than to BMP9). In some embodiments, the amino acid substitutions do not substantially change extracellular ActRIIB function (e.g., the ActRIIB variants are functionally equivalent to the wild-type extracellular ActRIIB). In some embodiments, the amino acid substitutions confer an ActRIIA property or activity on the ActRIIB variant (e.g., the ActRIIB variant has a longer half-life as an Fc fusion protein than WT extracellular ActRIIB-Fc). Preferably, the ActRIIB variants have one or more, two or more, or three or more of the above properties (e.g., reduced BMP9 binding and improved binding to activin A or B, myostatin, and/or GDF-11, or reduced BMP9 binding and functional equivalence to wild-type ActRIIB).

The ActRIIB variants (e.g., an extracellular ActRIIB variant having the sequence of any one of SEQ ID NOs: 269-283 (e.g., SEQ ID NOs: 270-283)) preferably have one or more amino acid substitutions that reduce BMP9 binding. In some embodiments, the amino acid substitution that reduces BMP9 binding is E75K (e.g., X₂₄ is K in SEQ ID NO: 269). In some embodiments, the amino acid substitutions that reduce BMP9 binding are Q69T and E70D (e.g., X₂₁ is T and X₂₂ is D in SEQ ID NO: 269). In some embodiments, the amino acid substitutions that reduce BMP9 binding are Q69D and E70T (e.g., X₂₁ is D and X₂₂ is T in SEQ ID NO: 269). In some embodiments, the amino acid substitutions that reduce BMP9 binding are T74K, E75K, E76D, N77S, and Q79E (e.g., X₂₃, X₂₄, X₂₅, X₂₆, and X₂₈ are K, K, D, S, and E, respectively, in SEQ ID NO: 269). In some embodiments, the ActRIIB variants have more than one of the aforementioned amino acid substitutions that reduce BMP9 binding (e.g., substitution E75K and substitutions Q69D and E70T, or substitution E75K and substitutions Q69T and E70D). In some embodiments, the ActRIIB variants have one or more amino acid substitutions that reduce BMP9 binding, and one or more additional amino acid substitutions. The additional amino acid substitutions may confer other beneficial properties, such as altered binding to activins or myostatin or improved activity. For example, amino acid substitutions T74K, E75K, E76D, N77S, and Q79E lead to a reduction in ActRIIB variant activity (e.g., compared to wild-type extracellular ActRIIB), but including additional substitutions S25T and S471; E31Y, E33D, and Q34K; or Y41F, R45K, and K56Q improves the effect of the ActRIIB variant. The additional amino acid substitutions may include one or more of substitutions I11L, Y12F, L19K, E20D, S25T, L27V, R29P, E31Y, E33D, Q34K, L38R, Y41F, R45K, S471, S48T, T50S, I51L, L531, K56Q and F631, T74K, E76D, N77S, Q79E, or F89M.

In some embodiments, a polypeptide described herein includes an extracellular ActRIIB variant having the sequence of SEQ ID NO: 269.

TABLE 5
Amino acid substitutions in an extracellular
ActRIIB variant having a sequence of
SEQ ID NO: 269

GRGEAETRECX₁X₂YNANWEX₃X₄RTNQX₅GX₆EX₇CX₈GX₉X₁₀DKRX₁₁H
CX₁₂ASWX₁₃NX₁₄X₁₅GX₁₆X₁₇EX₁₈VKX₁₉GCWLDDX₂₀NCYDRX₂₁X₂₂CV
AX₂₃X₂₄X₂₅X₂₆PX₂₇VYFCCCEGNX₂₈CNERFTHLPEAGGPEVTYEPPP
TAPT (SEQ ID NO: 269)

| | | | |
|---|---|---|---|
| X₁ | I or L | X₁₅ | S or T |
| X₂ | F or Y | X₁₆ | S or T |
| X₃ | L or K | X₁₇ | I or L |
| X₄ | D or E | X₁₈ | I or L |
| X₅ | T or S | X₁₉ | K or Q |
| X₆ | L or V | X₂₀ | F or I |
| X₇ | P or R | X₂₁ | Q, T, or D |
| X₈ | Y or E | X₂₂ | E, D, or T |
| X₉ | D or E | X₂₃ | K or T |
| X₁₀ | K or Q | X₂₄ | K or E |
| X₁₁ | R or L | X₂₅ | D or E |
| X₁ | Y or F | X₂₆ | S or N |
| X₁₃ | R or K | X₂₇ | E or Q |
| X₁₄ | S or I | X₂₈ | F or M |

In same embodiments, a polypeptide far use in a method described herein includes an extracellular ActRIIB variant having a sequence of any one of SEQ ID NOs: 270-283 (Table 6).

TABLE 6
Extracellular ActRIIB variants having the sequences of SEQ ID NOs: 270-283

| SEQ ID NO: | Amino Acid Sequence |
|---|---|
| 270 | GRGEAETRECIFYNANWEKDRTNQSGLEPCYGDQDKRRHCFASWKNSSGTIELVK |
| | QGCWLDDINCYDRQECVAKKDSPEVYFCCCEGNFCNERFTHLPEAGGPEVTYEP |
| | PPTAPT |
| 271 | GRGEAETRECIYYNANWELDRTNQSGLERCEGEQDKRLHCYASWRNSSGTIELVK |
| | KGCWLDDINCYDRQECVATKENPQVYFCCCEGNFCNERFTHLPEAGGPEVTYEPP |
| | PTAPT |
| 272 | GRGEAETRECIYYNANWELERTNQSGLERCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 273 | GRGEAETRECIYYNANWELERTNQTGLERCEGEQDKRLHCYASWRNISGTIELVK |
| | KGCWLDDFNCYDRQECVAKKDSPEVYFCCCEGNFCNERFTHLPEAGGPEVTYEP |
| | PPTAPT |
| 274 | GRGEAETRECIYYNANWELERTNQTGLERCEGEQDKRLHCYASWRNITGTIELVK |
| | KGCWLDDFNCYDRQECVAKKDSPEVYFCCCEGNFCNERFTHLPEAGGPEVTYEPP |
| | PTAPT |
| 275 | GRGEAETRECIYYNANWELERTNQSGLEPCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 276 | GRGEAETRECIYYNANWELERTNQSGLERCYGDKDKRLHCYASWRNSSGTIELVK |
| | KGCWLDDFNCYDRQECVAKKDSPEVYFCCCEGNFCNERFTHLPEAGGPEVTYEP |
| | PPTAPT |
| 277 | GRGEAETRECIYYNANWELERTNQSGLERCEGEQDKRLHCFASWKNSSGTIELVK |
| | QGCWLDDFNCYDRQECVAKKDSPEVYFCCCEGNFCNERFTHLPEAGGPEVTYEP |
| | PPTAPT |
| 278 | GRGEAETRECIFYNANWEKDRTNQSGLERCEGEQDKRLHCYASWRNSSGTIELVK |
| | KGCWLDDFNCYDRQECVAKKDSPEVYFCCCEGNFCNERFTHLPEAGGPEVTYEP |
| | PPTAPT |
| 279 | GRGEAETRECIYYNANWELERTNQSGLERCYGDQDKRRHCYASWRNSSGTIELV |
| | KKGCWLDDFNCYDRQECVAKKDSPEVYFCCCEGNFCNERFTHLPEAGGPEVTYE |
| | PPPTAPT |
| 280 | GRGEAETRECLYYNANWELERTNQSGVERCEGEKDKRLHCYASWRNSSGSLEIV |
| | KKGCWLDDFNCYDRTDCVATEENPQVYFCCCEGNMCNERFTHLPEAGGPEVTYE |
| | PPPTAPT |
| 281 | GRGEAETRECLYYNANWELERTNQSGVERCEGEKDKRLHCYASWRNSSGSLEIV |
| | KKGCWLDDFNCYDRDTCVATEENPQVYFCCCEGNMCNERFTHLPEAGGPEVTYE |
| | PPPTAPT |
| 282 | GRGEAETRECLYYNANWELERTNQSGVERCEGEKDKRLHCYASWRNSSGSLEIV |
| | KKGCWLDDFNCYDRTDCVATKENPQVYFCCCEGNMCNERFTHLPEAGGPEVTYE |
| | PPPTAPT |
| 283 | GRGEAETRECLYYNANWELERTNQSGVERCEGEKDKRLHCYASWRNSSGSLEIV |
| | KKGCWLDDFNCYDRDTCVATKENPQVYFCCCEGNMCNERFTHLPEAGGPEVTYE |
| | PPPTAPT |

In some embodiments, the extracellular ActRIIB variant has an N-terminal truncation of 1-7 amino acids (e.g., 1, 2, 3, 4, 5, 6, or 7 amino acids). The N-terminal truncation can involve the removal of 1-7 amino acids from the N-terminus of any of the ActRIIB variants shown in Tables 5 and 6. The N-terminal truncation can remove amino acids up to two amino acids before the first cysteine (e.g., the two amino acids before the first cysteine (RE) are retained in the N-terminally truncated ActRIIB variants).

In some embodiments, a polypeptide including an extracellular ActRIIB variant may further include an Fc domain monomer, which may be fused to the N- or C-terminus (e.g., C-terminus) of the extracellular ActRIIB variant by way of a linker or other covalent bonds. A polypeptide including an extracellular ActRIIB variant fused to an Fc domain monomer may form a dimer (e.g., homodimer or heterodimer) through the interaction between two Fc domain monomers, which combine to form an Fc domain in the dimer.

Furthermore, in some embodiments, a polypeptide including an ActRIIB variant described herein (e.g., an ActRIIB variant-Fc fusion protein) has a serum half-life of at least 7 days in humans. The polypeptide including an extracellular ActRIIB variant may bind to activin A with a K_{D} of 10 pM or higher.

In some embodiments, the polypeptide including an extracellular ActRIIB variant does not bind to BMP9 or activin A. In some embodiments, the polypeptide including an extracellular ActRIIB variant binds to activin A, activin B, and/or myostatin and exhibits reduced (e.g., weak) binding to BMP9. In some embodiments, the polypeptide including an extracellular ActRIIB variant does not substantially bind to human BMP9.

In some embodiments, the polypeptide including an extracellular ActRIIB variant may bind to human activin A with a K_{D} of about 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide including an extracellular ActRIIB variant may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). The polypeptide including an extracellular ActRIIB variant may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

### Fc Domains

In some embodiments, a polypeptide described herein may include an extracellular ActRII variant (e.g., an extracellular ActRII chimera or an extracellular ActRIIB variant) fused to an Fc domain monomer of an immunoglobulin or a fragment of an Fc domain to increase the serum half-life of the polypeptide. A polypeptide including an extracellular ActRII variant fused to an Fc domain monomer may form a dimer (e.g., homodimer or heterodimer) through the interaction between two Fc domain monomers, which form an Fc domain in the dimer. As conventionally known in the art, an Fc domain is the protein structure that is found at the C-terminus of an immunoglobulin. An Fc domain includes two Fc domain monomers that are dimerized by the interaction between the C_{H}3 antibody constant domains. A wild-type Fc domain forms the minimum structure that binds to an Fc receptor, e.g., FcγRI, FcγRIIa, FcγRIIb, FcγRIIIa, FcγRIIIb, FcγRIV. In some embodiments, an Fc domain may be mutated to lack effector functions, typical of a “dead” Fc domain. For example, an Fc domain may include specific amino acid substitutions that are known to minimize the interaction between the Fc domain and an Fcγ receptor. In some embodiments, an Fc domain is from an IgG1 antibody and includes amino acid substitutions L234A, L235A, and G237A. In some embodiments, an Fc domain is from an IgG1 antibody and includes amino acid substitutions D265A, K322A, and N434A. The aforementioned amino acid positions are defined according to Kabat (Sequences of Proteins of Immunological Interest, 5th Ed. Public Health Service, National Institutes of Health, Bethesda, MD. (1991)). The Kabat numbering of amino acid residues may be determined for a given antibody by alignment at regions of homology of the sequence of the antibody with a “standard” Kabat numbered sequence. Furthermore, in some embodiments, an Fc domain does not induce any immune system-related response. For example, the Fc domain in a dimer of a polypeptide including an extracellular ActRII variant fused to an Fc domain monomer may be modified to reduce the interaction or binding between the Fc domain and an Fcγ receptor. The sequence of an Fc domain monomer that may be fused to an extracellular ActRII variant (e.g., an extracellular ActRII chimera or an extracellular ActRIIB variant) is shown below (SEQ ID NO: 48):

THTCPPCPAPELLGGPSVFLFPPKPKDTLMISRTPEVTCVVVDVSHE
DPEVKFNWYVDGVEVHNAKTKPREEQYNSTYRVVSVLTVLHQDWLNG
KEYKCKVSNKALPVPIEKTISKAKGQPREPQVYTLPPSREEMTKNQV
SLTCLVKGFYPSDIAVEWESNGQPENNYKTTPPVLDSDGPFFLYSKL
TVDKSRWQQGNVFSCSVMHEALHNHYTQKSLSLSPGK

The sequence of a wild-type Fc domain that may be fused to an extracellular ActRII variant (e.g., an extracellular ActRII chimera or an extracellular ActRIIB variant) is shown below is shown below in SEQ ID NO: 100:

DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWL
NGKEYKCKVSNKALPAPIEKTISKAKGQPREPQVYTLPPSRDELTKN
QVSLTCLVKGFYPSDIAVEWESNGQPENNYKTTPPVLDSDGSFFLYS
KLTVDKSRWQQGNVFSCSVMHEALHNHYTQKSLSLSPGK

In some embodiments, the Fc domain monomer fused to an extracellular ActRII variant lacks a terminal lysine. An exemplary sequence for a wild-type Fc domain lacking a terminal lysine is provided below (SEQ ID NO: 264):

DKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWL
NGKEYKCKVSNKALPAPIEKTISKAKGQPREPQVYTLPPSRDELTKN
QVSLTCLVKGFYPSDIAVEWESNGQPENNYKTTPPVLDSDGSFFLYS
KLTVDKSRWQQGNVFSCSVMHEALHNHYTQKSLSLSPG

In some embodiments, an Fc domain is from an IgG1 antibody and includes amino acid substitutions L12A, L13A, and G15A, relative to the sequence of SEQ ID NO: 48. In some embodiments, an Fc domain is from an IgG1 antibody and includes amino acid substitutions D43A, K100A, and N212A, relative to the sequence of SEQ ID NO: 48. In some embodiments, the terminal lysine is absent from the Fc domain monomer having the sequence of SEQ ID NO: 48 or SEQ ID NO: 100. In some embodiments, an extracellular ActRII variant described herein (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) may be fused to the N- or C-terminus of an Fc domain monomer (e.g., SEQ ID NO: 48, SEQ ID NO: 100, or SEQ ID NO: 264) through conventional genetic or chemical means, e.g., chemical conjugation. If desired, a linker (e.g., a spacer) can be inserted between the extracellular ActRII variant and the Fc domain monomer. The Fc domain monomer can be fused to the N- or C-terminus (e.g., C-terminus) of the extracellular ActRII variant. The Fc domain monomer can be of immunoglobulin antibody isotype IgG, IgE, IgM, IgA, or IgD. Additionally, the Fc domain monomer can be an IgG subtype (e.g., IgG1, IgG2a, IgG2b, IgG3, or IgG4). In some embodiments, the Fc domain monomer is an IgG1 Fc domain monomer (e.g., a human IgG1 Fc domain monomer).

In some embodiments, the Fc domain contains one or more amino acid substitutions that reduce or inhibit Fc domain dimerization. In some embodiments, the Fc domain contains a hinge domain. The Fc domain can be of immunoglobulin antibody isotype IgG, IgE, IgM, IgA, or IgD. Additionally, the Fc domain can be an IgG subtype (e.g., IgG1, IgG2a, IgG2b, IgG3, or IgG4). The Fc domain can also be a non-naturally occurring Fc domain, e.g., a recombinant Fc domain.

Methods of engineering Fc domains that have reduced dimerization are known in the art. In some embodiments, one or more amino acids with large sidechains (e.g., tyrosine or tryptophan) may be introduced to the C_{H}3-C_{H}3 dimer interface to hinder dimer formation due to steric clash. In other embodiments, one or more amino acids with small sidechains (e.g., alanine, valine, or threonine) may be introduced to the C_{H}3-C_{H}3 dimer interface to remove favorable interactions. Methods of introducing amino acids with large or small sidechains in the C_{H}3 domain are described in, e.g., Ying et al. (J Biol Chem. 287:19399-19408, 2012), U.S. Patent Publication No. 2006/0074225, U.S. Pat. Nos. 8,216,805 and 5,731,168, Ridgway et al. (Protein Eng. 9:617-612, 1996), Atwell et al. (J Mol Biol. 270:26-35, 1997), and Merchant et al. (Nat Biotechnol. 16:677-681, 1998), all of which are incorporated herein by reference in their entireties.

In yet other embodiments, one or more amino acid residues in the C_{H}3 domain that make up the C_{H}3-C_{H}3 interface between two Fc domains are replaced with positively charged amino acid residues (e.g., lysine, arginine, or histidine) or negatively charged amino acid residues (e.g., aspartic acid or glutamic acid) such that the interaction becomes electrostatically unfavorable depending on the specific charged amino acids introduced. Methods of introducing charged amino acids in the C_{H}3 domain to disfavor or prevent dimer formation are described in, e.g., Ying et al. (J Biol Chem. 287:19399-19408, 2012), U.S. Patent Publication Nos. 2006/0074225, 2012/0244578, and 2014/0024111, all of which are incorporated herein by reference in their entireties.

In some embodiments of the invention, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y407I, K409E, K409D, K409T, and K409I, relative to the sequence of human IgG1. In some embodiments, the terminal lysine is absent from the Fc domain amino acid sequence. In one particular embodiment, an Fc domain includes the amino acid substitution T366W, relative to the sequence of human IgG1.

### Linkers

A polypeptide described herein may include an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) fused to an Fc domain monomer by way of a linker, which can increase stability of the polypeptide. In the present invention, a linker between an Fc domain monomer (e.g., the sequence of SEQ ID NO: 48, SEQ ID NO: 100, or SEQ ID NO: 264) and an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) can be an amino acid spacer including 1-200 amino acids. Suitable peptide spacers are known in the art, and include, for example, peptide linkers containing flexible amino acid residues such as glycine, alanine, and serine. In some embodiments, a spacer can contain motifs, e.g., multiple or repeating motifs, of GA, GS, GG, GGA, GGS, GGG, GGGA (SEQ ID NO: 49), GGGS (SEQ ID NO: 50), GGGG (SEQ ID NO: 51), GGGGA (SEQ ID NO: 52), GGGGS (SEQ ID NO: 53), GGGGG (SEQ ID NO: 54), GGAG (SEQ ID NO: 55), GGSG (SEQ ID NO: 56), AGGG (SEQ ID NO: 57), or SGGG (SEQ ID NO: 58). In some embodiments, a spacer can contain 2 to 12 amino acids including motifs of GA or GS, e.g., GA, GS, GAGA (SEQ ID NO: 59), GSGS (SEQ ID NO: 60), GAGAGA (SEQ ID NO: 61), GSGSGS (SEQ ID NO: 62), GAGAGAGA (SEQ ID NO: 63), GSGSGSGS (SEQ ID NO: 64), GAGAGAGAGA (SEQ ID NO: 65), GSGSGSGSGS (SEQ ID NO: 66), GAGAGAGAGAGA (SEQ ID NO: 67), and GSGSGSGSGSGS (SEQ ID NO: 68). In some embodiments, a spacer can contain 3 to 12 amino acids including motifs of GGA or GGS, e.g., GGA, GGS, GGAGGA (SEQ ID NO: 69), GGSGGS (SEQ ID NO: 70), GGAGGAGGA (SEQ ID NO: 71), GGSGGSGGS (SEQ ID NO: 72), GGAGGAGGAGGA (SEQ ID NO: 73), and GGSGGSGGSGGS (SEQ ID NO: 74). In yet some embodiments, a spacer can contain 4 to 12 amino acids including motifs of GGAG (SEQ ID NO: 55), GGSG (SEQ ID NO: 56), e.g., GGAG (SEQ ID NO: 55), GGSG (SEQ ID NO: 56), GGAGGGAG (SEQ ID NO: 75), GGSGGGSG (SEQ ID NO: 76), GGAGGGAGGGAG (SEQ ID NO: 77), and GGSGGGSGGGSG (SEQ ID NO: 78). In some embodiments, a spacer can contain motifs of GGGGA (SEQ ID NO: 52) or GGGGS (SEQ ID NO: 53), e.g., GGGGAGGGGAGGGGA (SEQ ID NO: 79) and GGGGSGGGGSGGGGS (SEQ ID NO: 80). In some embodiments of the invention, an amino acid spacer between an Fc domain monomer and an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) may be GGG, GGGA (SEQ ID NO: 49), GGGG (SEQ ID NO: 51), GGGAG (SEQ ID NO: 81), GGGAGG (SEQ ID NO: 82), or GGGAGGG (SEQ ID NO: 83).

In some embodiments, a spacer can also contain amino acids other than glycine, alanine, and serine, e.g., AAAL (SEQ ID NO: 84), AAAK (SEQ ID NO: 85), AAAR (SEQ ID NO: 86), EGKSSGSGSESKST (SEQ ID NO: 87), GSAGSAAGSGEF (SEQ ID NO: 88), AEAAAKEAAAKA (SEQ ID NO: 89), KESGSVSSEQLAQFRSLD (SEQ ID NO: 90), GENLYFQSGG (SEQ ID NO: 91), SACYCELS (SEQ ID NO: 92), RSIAT (SEQ ID NO: 93), RPACKIPNDLKQKVMNH (SEQ ID NO: 94), GGSAGGSGSGSSGGSSGASGTGTAGGTGSGSGTGSG (SEQ ID NO: 95), AAANSSIDLISVPVDSR (SEQ ID NO: 96), or GGSGGGSEGGGSEGGGSEGGGSEGGGSEGGGSGGGS (SEQ ID NO: 97). In some embodiments, a spacer can contain motifs, e.g., multiple or repeating motifs, of EAAAK (SEQ ID NO: 98). In some embodiments, a spacer can contain motifs, e.g., multiple or repeating motifs, of proline-rich sequences such as (XP)ₙ, in which X may be any amino acid (e.g., A, K, or E) and n is from 1-5, and PAPAP (SEQ ID NO: 99).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, the linker between an Fc domain monomer (e.g., the sequence of SEQ ID NO: 48, SEQ ID NO: 100, or SEQ ID NO: 264) and an extracellular ActRII variant described herein (e.g., or an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) is an amino acid spacer having the sequence GGG. For example, a polypeptide of the invention can contain an extracellular ActRII chimera (e.g., any one of SEQ ID NOs: 22-43) fused to an Fc domain monomer (e.g., SEQ ID NO: 100) by a GGG linker. An exemplary polypeptide containing an ActRII chimera of SEQ ID NO: 41, a GGG linker, and an Fc domain monomer (SEQ ID NO: 100) is provided below (SEQ ID NO: 216):

GAILGRSETQECIYYNANWELERTNQSGLERCEGEQDKRLHCYASWR
NSSGTIEIVKQGCWLDDFNCYDRTDCVEKKDSPQVYFCCCEGNMCNE
KFSYFPEMEVTQPTSGGGDKTHTCPPCPAPELLGGPSVFLFPPKPKD
TLMISRTPEVTCVVVDVSHEDPEVKFNWYVDGVEVHNAKTKPREEQY
NSTYRVVSVLTVLHQDWLNGKEYKCKVSNKALPAPIEKTISKAKGQP
REPQVYTLPPSRDELTKNQVSLTCLVKGFYPSDIAVEWESNGQPENN
YKTTPPVLDSDGSFFLYSKLTVDKSRWQQGNVFSCSVMHEALHNHYT
QKSLSLSPGK

In another example, a polypeptide of the invention can contain an extracellular ActRIIB variant (e.g., any one of SEQ ID NOs: 270-283) fused to an Fc domain monomer (e.g., SEQ ID NO: 100 or SEQ ID NO: 264) by a GGG linker. An exemplary polypeptide containing an ActRIIB variant of SEQ ID NO: 283, a GGG linker, and an Fc domain monomer (SEQ ID NO: 100) is provided below (SEQ ID NO: 284):

GRGEAETRECLYYNANWELERTNQSGVERCEGEKDKRLHCYASWRNS
SGSLEIVKKGCWLDDFNCYDRDTCVATKENPQVYFCCCEGNMCNERF
THLPEAGGPEVTYEPPPTAPTGGGDKTHTCPPCPAPELLGGPSVFLF
PPKPKDTLMISRTPEVTCVVVDVSHEDPEVKFNWYVDGVEVHNAKTK
PREEQYNSTYRVVSVLTVLHQDWLNGKEYKCKVSNKALPAPIEKTIS
KAKGQPREPQVYTLPPSRDELTKNQVSLTCLVKGFYPSDIAVEWESN
GQPENNYKTTPPVLDSDGSFFLYSKLTVDKSRWQQGNVFSCSVMHEA
LHNHYTQKSLSLSPGK

The C-terminal Lys339 of the polypeptide of SEQ ID NO: 216 (the C-terminal Lys in the Fc region of SEQ ID NO: 216) and the C-terminal Lys345 of the polypeptide of SEQ ID NO: 284 (the C-terminal Lys in the Fc region of SEQ ID NO: 284) may or may not be present, without affecting the structure or stability of the polypeptide. The disclosure specifically contemplates SEQ ID NO: 216 that does not include the C-terminal Lys corresponding to Lys339 and SEQ ID NO: 284 that does not include the C-terminal Lys corresponding to Lys345. The polypeptide of SEQ ID NO: 216 may be expressed including a C-terminal Lys339 which then may be proteolytically cleaved upon expression of the polypeptide, and the polypeptide of SEQ ID NO: 284 may be expressed including a C-terminal Lys345 which then may be proteolytically cleaved upon expression of the polypeptide (e.g., the polypeptides of SEQ ID NO: 216 and SEQ ID NO: 284 are expressed using nucleic acid constructs encoding the polypeptide including a C-terminal lysine residue). The polypeptides of SEQ ID NO: 216 and SEQ ID NO: 284 may also be expressed without including the C-terminal Lys339 and the C-terminal Lys345, respectively.

### Vectors, Host Cells, and Protein Production

The polypeptides described herein can be produced from a host cell. A host cell refers to a vehicle that includes the necessary cellular components, e.g., organelles, needed to express the polypeptides and fusion polypeptides described herein from their corresponding nucleic acids. The nucleic acids may be included in nucleic acid vectors that can be introduced into the host cell by conventional techniques known in the art (e.g., transformation, transfection, electroporation, calcium phosphate precipitation, direct microinjection, infection, or the like). The choice of nucleic acid vectors depends in part on the host cells to be used. Generally, preferred host cells are of either eukaryotic (e.g., mammalian) or prokaryotic (e.g., bacterial) origin.

### Nucleic Acid Vector Construction and Host Cells

A nucleic acid sequence encoding the amino acid sequence of a polypeptide described herein may be prepared by a variety of methods known in the art. These methods include, but are not limited to, oligonucleotide-mediated (or site-directed) mutagenesis, PCR mutagenesis, ligation, and overlap extension PCR. A nucleic acid molecule encoding a polypeptide described herein may be obtained using standard techniques, e.g., gene synthesis. Alternatively, a nucleic acid molecule encoding a wild-type extracellular ActRIIA or ActRIIB may be mutated to include specific amino acid substitutions using standard techniques in the art, e.g., QuikChange™ mutagenesis. Nucleic acid molecules can be synthesized using a nucleotide synthesizer or PCR techniques.

A nucleic acid sequence encoding a polypeptide described herein may be inserted into a vector capable of replicating and expressing the nucleic acid molecule in prokaryotic or eukaryotic host cells. Many vectors are available in the art and can be used for the purpose of the invention. Each vector may include various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence.

In some embodiments, mammalian cells may be used as host cells for the invention. Examples of mammalian cell types include, but are not limited to, human embryonic kidney (HEK) (e.g., HEK293, HEK 293F), Chinese hamster ovary (CHO), HeLa, COS, PC3, Vero, MC3T3, NSO, Sp2/0, VERY, BHK, MDCK, W138, BT483, Hs578T, HTB2, BT20, T47D, NSO (a murine myeloma cell line that does not endogenously produce any immunoglobulin chains), CRL7O3O, and HsS78Bst cells. In some embodiments, E. coli cells may also be used as host cells for the invention. Examples of E. coli strains include, but are not limited to, E. coli 294 (ATCC® 31,446), E. coli A 1776 (ATCC® 31,537, E. coli BL21 (DE3) (ATCC® BAA-1025), and E. coli RV308 (ATCC® 31,608). Different host cells have characteristic and specific mechanisms for the posttranslational processing and modification of protein products (e.g., glycosylation). Appropriate cell lines or host systems may be chosen to ensure the correct modification and processing of the polypeptide expressed. The above-described expression vectors may be introduced into appropriate host cells using conventional techniques in the art, e.g., transformation, transfection, electroporation, calcium phosphate precipitation, and direct microinjection. Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 and Vladimir Voynov and Justin A. Caravella (eds.) Therapeutic Proteins: Methods and Protocols (Methods in Molecular Biology) Humana Press; 2nd ed. 2012.

### Protein Production, Recovery, and Purification

Host cells used to produce the polypeptides described herein may be grown in media known in the art and suitable for culturing of the selected host cells. Examples of suitable media for mammalian host cells include Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO2 levels, such as 5 to 10%. The pH of the medium is generally from about 6.8 to 7.4, e.g., 7.0, depending mainly on the host organism. If an inducible promoter is used in the expression vector of the invention, protein expression is induced under conditions suitable for the activation of the promoter.

In some embodiments, depending on the expression vector and the host cells used, the expressed protein may be secreted from the host cells (e.g., mammalian host cells) into the cell culture media. Protein recovery may involve filtering the cell culture media to remove cell debris. The proteins may be further purified. A polypeptide described herein may be purified by any method known in the art of protein purification, for example, by chromatography (e.g., ion exchange, affinity, and size-exclusion column chromatography), centrifugation, differential solubility, or by any other standard technique for the purification of proteins. For example, the protein can be isolated and purified by appropriately selecting and combining affinity columns such as Protein A column (e.g., POROS Protein A chromatography) with chromatography columns (e.g., POROS HS-50 cation exchange chromatography), filtration, ultra filtration, salting-out and dialysis procedures.

In other embodiments, host cells may be disrupted, e.g., by osmotic shock, sonication, or lysis, to recover the expressed protein. Once the cells are disrupted, cell debris may be removed by centrifugation or filtration. In some instances, a polypeptide can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from influenza hemagglutinin protein (Wilson et al., Cell 37:767, 1984).

Alternatively, the polypeptides described herein can be produced by the cells of a subject (e.g., a human), e.g., in the context of gene therapy, by administering a vector (such as a viral vector (e.g., a retroviral vector, adenoviral vector, poxviral vector (e.g., vaccinia viral vector, such as Modified Vaccinia Ankara (MVA)), adeno-associated viral vector, and alphaviral vector)) containing a nucleic acid molecule encoding the polypeptide described herein. The vector, once inside a cell of the subject (e.g., by transformation, transfection, electroporation, calcium phosphate precipitation, direct microinjection, infection, etc.) will promote expression of the polypeptide, which is then secreted from the cell. If treatment of a disease or disorder is the desired outcome, no further action may be required. If collection of the protein is desired, blood may be collected from the subject and the protein purified from the blood by methods known in the art.

### Pharmaceutical Compositions and Preparations

The invention features pharmaceutical compositions that include the polypeptides described herein (e.g., a polypeptide including an extracellular ActRII chimera (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)). In some embodiments, a pharmaceutical composition of the invention includes a polypeptide including an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) fused to an Fc domain monomer as the therapeutic protein. In some embodiments, a pharmaceutical composition including a polypeptide described herein may be used in combination with other agents (e.g., therapeutic biologics and/or small molecules) or compositions in a therapy. In addition to a therapeutically effective amount of the polypeptide, the pharmaceutical composition may include one or more pharmaceutically acceptable carriers or excipients, which can be formulated by methods known to those skilled in the art. In some embodiments, a pharmaceutical composition of the invention includes a nucleic acid molecule (DNA or RNA, e.g., mRNA) encoding a polypeptide of the invention, or a vector containing such a nucleic acid molecule.

Acceptable carriers and excipients in the pharmaceutical compositions are nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, arginine, and lysine, and carbohydrates such as glucose, mannose, sucrose, and sorbitol. Pharmaceutical compositions of the invention can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water, physiological saline, and cell culture media (e.g., Dulbecco's Modified Eagle Medium (DMEM), α-Modified Eagles Medium (α-MEM), F-12 medium). Formulation methods are known in the art, see e.g., Banga (ed.) Therapeutic Peptides and Proteins: Formulation, Processing and Delivery Systems (3rd ed.) Taylor & Francis Group, CRC Press (2015).

The pharmaceutical compositions may be prepared in microcapsules, such as hydroxylmethylcellulose or gelatin-microcapsule and poly-(methylmethacrylate) microcapsule. The pharmaceutical compositions may also be prepared in other drug delivery systems such as liposomes, albumin microspheres, microemulsions, nanoparticles, and nanocapsules. Such techniques are described in Remington: The Science and Practice of Pharmacy 22^{nd} edition (2012). The pharmaceutical compositions to be used for in vivo administration must be sterile. This is readily accomplished by filtration through sterile filtration membranes.

The pharmaceutical compositions may also be prepared as a sustained-release formulation. Suitable examples of sustained-release preparations include semipermeable matrices of solid hydrophobic polymers containing the polypeptides of the invention. Examples of sustained release matrices include polyesters, hydrogels, polylactides, copolymers of L-glutamic acid and γ ethyl-L-glutamate, non-degradable ethylene-vinyl acetate, degradable lactic acid-glycolic acid copolymers such as LUPRON DEPOT™, and poly-D-(−)-3-hydroxybutyric acid. Some sustained-release formulations enable release of molecules over a few months, e.g., one to six months, while other formulations release pharmaceutical compositions of the invention for shorter time periods, e.g., days to weeks.

The pharmaceutical composition may be formed in a unit dose form as needed. The amount of active component, e.g., a polypeptide of the invention, included in the pharmaceutical preparations is such that a suitable dose within the designated range is provided (e.g., a dose within the range of 0.01-100 mg/kg of body weight).

The pharmaceutical composition for gene therapy can be in an acceptable diluent or can include a slow-release matrix in which the gene delivery vehicle is imbedded. If hydrodynamic injection is used as the delivery method, the pharmaceutical composition containing a nucleic acid molecule encoding a polypeptide described herein or a vector (e.g., a viral vector) containing the nucleic acid molecule is delivered rapidly in a large fluid volume intravenously. Vectors that may be used as in vivo gene delivery vehicle include, but are not limited to, retroviral vectors, adenoviral vectors, poxviral vectors (e.g., vaccinia viral vectors, such as Modified Vaccinia Ankara), adeno-associated viral vectors, and alphaviral vectors.

### Routes, Dosage, and Administration

Pharmaceutical compositions that include the polypeptides described herein as the therapeutic proteins may be formulated for, e.g., intravenous administration, parenteral administration, subcutaneous administration, intramuscular administration, intra-arterial administration, intrathecal administration, or intraperitoneal administration. The pharmaceutical composition may also be formulated for, or administered via, oral, nasal, spray, aerosol, rectal, or vaginal administration. For injectable formulations, various effective pharmaceutical carriers are known in the art. See, e.g., ASHP Handbook on Injectable Drugs, Toissel, 18th ed. (2014).

In some embodiments, a pharmaceutical composition that includes a nucleic acid molecule encoding a polypeptide described herein or a vector containing such nucleic acid molecule may be administered by way of gene delivery. Methods of gene delivery are well-known to one of skill in the art. Vectors that may be used for in vivo gene delivery and expression include, but are not limited to, retroviral vectors, adenoviral vectors, poxviral vectors (e.g., vaccinia viral vectors, such as Modified Vaccinia Ankara (MVA)), adeno-associated viral vectors, and alphaviral vectors. In some embodiments, mRNA molecules encoding polypeptides described herein may be administered directly to a subject.

In some embodiments of the present invention, nucleic acid molecules encoding a polypeptide described herein or vectors containing such nucleic acid molecules may be administered using a hydrodynamic injection platform. In the hydrodynamic injection method, a nucleic acid molecule encoding a polypeptide described herein is put under the control of a strong promoter in an engineered plasmid (e.g., a viral plasmid). The plasmid is often delivered rapidly in a large fluid volume intravenously. Hydrodynamic injection uses controlled hydrodynamic pressure in veins to enhance cell permeability such that the elevated pressure from the rapid injection of the large fluid volume results in fluid and plasmid extravasation from the vein. The expression of the nucleic acid molecule is driven primarily by the liver. In mice, hydrodynamic injection is often performed by injection of the plasmid into the tail vein. In certain embodiments, mRNA molecules encoding a polypeptide described herein may be administered using hydrodynamic injection.

The dosage of the pharmaceutical compositions depends on factors including the route of administration, the disease to be treated, and physical characteristics, e.g., age, weight, general health, of the subject. A pharmaceutical composition may include a dosage of a polypeptide described herein ranging from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg) and, in a more specific embodiment, about 0.1 to about 30 mg/kg and, in a more specific embodiment, about 0.3 to about 30 mg/kg. The dosage may be adapted by the physician in accordance with conventional factors such as the extent of the disease and different parameters of the subject.

The pharmaceutical compositions are administered in a manner compatible with the dosage formulation and in such amount as is therapeutically effective to result in an improvement or remediation of the symptoms. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 0.1-100 mg/kg, e.g., 0.5-50 mg/kg. Pharmaceutical compositions that include a polypeptide of the invention may be administered to a subject in need thereof, for example, one or more times (e.g., 1-10 times or more) daily, weekly, biweekly, every four weeks, monthly, every eight weeks, bimonthly, every 12 weeks, quarterly, every sixteen weeks biannually, annually, or as medically necessary. In some embodiments, pharmaceutical compositions that include a polypeptide described herein may be administered to a subject in need thereof weekly, biweekly, every four weeks, monthly, every eight weeks, bimonthly, every twelve weeks, quarterly, or every sixteen weeks. Dosages may be provided in either a single or multiple dosage regimens. The timing between administrations may increase as the medical condition improves or decrease as the health of the patient declines.

### Methods of Treatment

The ActRII variants described herein have improved properties compared to endogenous ActRIIA and ActRIIB. For example, the ActRIIB variants generated by introducing residues from ActRIIA into ActRIIB may retain the beneficial properties of ActRIIB, such high binding affinity to activins A and B, and gain some of the beneficial properties of ActRIIA, such as reduced binding affinity to BMP9 and longer serum half-life as an Fc fusion protein. In addition, the ActRII chimeras generated by combining extracellular portions of ActRIIA and ActRIIB may possess beneficial properties of both ActRIIB (e.g., strong binding affinity to activins A and B) and ActRIIA (e.g., reduced binding affinity to BMP9 and/or longer serum half-life as an Fc fusion protein (e.g., compared to ActRIIB-Fc)). These ActRIIB variant and ActRII chimera properties make for a useful therapeutic that can compete with endogenous activin receptors for ligand binding. As the ActRIIB variants and ActRII chimeras contain the extracellular portion of the receptor, they will be soluble and able to bind to and sequester ligands (e.g., activins A and B, myostatin, GDF11) without activating intracellular signaling pathways. Based on the discovery that administration of a polypeptide containing an ActRIIB variant described herein to human subjects led to a reduction in markers of fibrosis and inflammation, polypeptides containing the ActRII variants described herein (e.g., ActRIIB variants or ActRII chimeras) can be used therapeutically to treat subjects with Alport syndrome, which features kidney fibrosis and kidney inflammation. Glomerular filtration rate (GFR), as estimated by creatinine clearance, is inversely correlated with cortical interstitial volume fraction, a measure of fibrosis of the renal interstitium. Creatinine clearance is maintained in the normal range until cortical interstitial fibrosis increases above the upper limit of the normal range, suggesting that suppression of renal interstitial fibrosis will protect kidney function in Alport patients.

The polypeptides described herein (e.g., a polypeptide including an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283))) can be used to treat a subject having Alport syndrome. The subject may have X-linked Alport syndrome, autosomal recessive Alport syndrome, or autosomal dominant Alport syndrome. In some embodiments, the subject has a mutation in the gene encoding the alpha 3 chain of type IV collagen (COL4A3), a mutation in the gene encoding the alpha 4 chain of type IV collagen (COL4A4), a mutation in the gene encoding the alpha 5 chain of type IV collagen (COL4A5), or a mutation in both COL4A3 and COL4A4. In some embodiments, the subject is male. In some embodiments, the subject is female. In some embodiments, the subject is an adult (e.g., 18 years of age or older, such as 20, 30, 40, 50, 60, or 70 years of age or older). In some embodiments, the subject is a pediatric subject (e.g., younger than 18 years of age, such as 15 years of age, 12 years of age, 10 years of age, or younger). In some embodiments, the subject is identified as having Alport syndrome prior to treatment with an ActRII variant described herein. In some embodiments, the method includes a step of identifying the subject as having Alport syndrome prior to treatment with an ActRII variant described herein. The subject may be identified as having Alport syndrome based on one or more of genetic testing (e.g., for a mutation in COL4A3, COL4A4, or COL4A5), tissue biopsy (e.g., of kidney or skin, e.g., to evaluate ultrastructural abnormalities), family history, urinalysis (for detecting hematuria and proteinuria), hematologic studies (for assessing renal insufficiency), audiometry (for detecting sensorineural hearing loss), ophthalmic examination (for detecting ocular abnormalities), and renal ultrasonography. Subjects with Alport syndrome have also been found to show aberrant urine levels of ADAM8, fibronectin, myosin 10, MMP-2, MMP-9, and podocin compared to normal control subjects, and levels of various biomarker combinations in urine (e.g., combinations of three (fibronectin, myosin 10, and MMP-2 or MMP-9) or two (myosin 10, and MMP-2 or MMP-9) biomarkers) can be used in the diagnosis of Alport syndrome.

A subject that may be treated as described herein may have mild renal impairment (e.g., a GFR from 60 to 89 mL/min/1.73 m²), moderate renal impairment, (e.g., a GFR from 30 to 59 mL/min/1.73 m²), or severe renal impairment (e.g., a GFR from 15 to 29 mL/min/1.73 m²) at the time of treatment initiation. The subject may have an elevated level of proteinuria (e.g., greater than or equal to 200 mg protein per day (24 h urine protein)), an elevated protein-to-creatinine ratio (PCR) (e.g., greater than or equal to 300 mg/g creatinine), and/or an elevated albumin-to-creatinine ratio (ACR) (e.g., greater than or equal to 200 mg/g creatinine). The subject may also have elevated levels of certain proteins in serum or urine, such as N-acetyl-β-D-glucosaminidase (NAG), neutrophil gelatinase-associated lipocalin (NGAL), kidney injury molecule-1 (KIM-1), interleukin-18 (IL-18), connective tissue growth factor (CTGF), monocyte chemoattractant protein 1 (MCP1), collagen IV (Col IV) fragments, collagen III (Col III) fragments, or podocyte proteins (e.g., markers of podocyte injury, such as nephrin and podocin), and/or elevated levels of certain proteins in blood, such as cystatin C, β-trace protein (BTP), or beta-2-microglobulin (B2M). In some embodiments, the subject has elevated blood urea nitrogen or elevated creatinine in blood.

The methods described herein may also include a step of assessing GFR or eGFR, kidney fibrosis, kidney inflammation, PCR, ACR, UACR (urinary albumin-creatinine-ratio), hematuria, proteinuria, albuminuria, the level of certain proteins in the urine, serum, or blood (e.g., the proteins described hereinabove), blood urea nitrogen, or creatinine in blood prior to treatment with or administration of the compositions described herein. The estimated GFR may be calculated based on serum creatinine values, e.g., using the Chronic Kidney Disease Epidemiology Collaboration (CKD-EPI) equation, the Cockcroft-Gault formula, or the Modification of Diet in Renal Disease (MDRD) formula, which are all known in the art. These assessments can also be performed after treatment with or administration of the compositions described herein (e.g., after the administration of one or more doses of a composition described herein, or one month, two months, three months, four months, six months, one year or longer after treatment initiation with a composition described herein.

The compositions described herein are administered in an amount sufficient to treat Alport syndrome, improve kidney function, improve GFR or eGFR, reduce kidney fibrosis (e.g., tubulointerstitial fibrosis), reduce glomerulosclerosis, or reduce kidney inflammation (i.e., nephritis, such as interstitial nephritis or glomerulonephritis). In some embodiments, the methods described herein slow or inhibit the progression of kidney fibrosis, delay the development of kidney fibrosis, slow or inhibit the progression of glomerulosclerosis, delay the development of glomerulosclerosis, slow or inhibit the progression of kidney inflammation, slow or inhibit a decline in kidney function, delay or prevent the onset of end stage renal disease, delay or prevent the need for dialysis, delay or prevent the need for continuous renal replacement therapy, or delay or prevent the need for a kidney transplant. Kidney function may be assessed by measuring GFR and improving kidney function or slowing or inhibiting a decline in kidney function may result in improving GFR or slowing or stopping a progressive decline in GFR. Kidney function may also be assessed by determining PCR, ACR, or UACR, and improving kidney function or slowing or inhibiting a decline in kidney function may result in improving (i.e., reducing) the PCR, ACR or UACR or slowing or stopping a progressive increase in PCR, ACR or UACR. Kidney function may also be assessed by measuring proteins present in the urine, serum, or blood. For example, kidney function may be assessed by measuring NAG, NGAL, KIM-1, IL-18, CTGF, MCP1, Col IV fragments, Col III fragment levels, nephrin, and/or podocin in the urine or serum of a subject, or measuring cystatin C, BTP, or B2M in the blood of a subject, and an improvement in kidney function may lead to reduced levels of one or more of these proteins in urine, serum, or blood. The proteins may be quantified, for example, by enzyme-linked immunosorbent assay (ELISA), or radioimmunoassay (RIA) using commercially available kits. Kidney function may also be assessed by measuring blood urea nitrogen, creatinine in the blood, creatinine clearance, or proteinuria, and an improvement in kidney function may be indicated by reduced blood urea nitrogen, reduced creatinine in the blood, improved creatinine clearance, or reduced proteinuria. In some embodiments, the methods described herein lead to normalization of one or more of ADAM8, fibronectin, myosin 10, MMP-2, MMP-9, and podocin urine levels, such as normalization by at least 10% compared to untreated subjects with Alport syndrome. In some embodiments, the method treats, prevents, delays the onset of, slows the progression of, or reduces the risk of hematuria, proteinuria, albuminuria, or a decline in GFR. In some embodiments, the methods described herein improve life expectancy for a subject with Alport syndrome. These outcomes can be assessed by comparing measurements obtained after treatment to measurements taken prior to treatment or by comparing measurements obtained after treatment to measurements from subjects with Alport syndrome who were not treated with a composition described herein. These effects may occur, for example, within 1 week, 2 weeks, 3 weeks, 4 weeks, 5 weeks, 6 weeks, 7 weeks, 8 weeks, 9 weeks, 10 weeks, 15 weeks, 20 weeks, 25 weeks, or more, following administration of the compositions described herein. The patient may be evaluated 1 month, 2 months, 3 months, 4 months, 5 months, 6 months, or more following administration of the composition depending on the dose and route of administration used for treatment. Depending on the outcome of the evaluation, the patient may receive additional treatments.

In some embodiments, the methods described herein (e.g., the methods of treating a subject with Alport syndrome) do not cause any vascular complications in the subject, such as increased vascular permeability or leakage.

In any of the methods described herein, a polypeptide including an extracellular ActRII chimera (e.g., an extracellular ActRII chimera having the sequence of any one of SEQ ID NOs: 1-43 (e.g., SEQ ID NOs: 22-43)) that further includes a C-terminal extension of one to six amino acids (e.g., 1, 2, 3, 4, 5, 6 or more amino acids from extracellular ActRIIA or ActRIIB) may be used as the therapeutic protein. In any of the methods described herein, a dimer (e.g., homodimer or heterodimer) formed by the interaction of two Fc domain monomers that are each fused to a polypeptide including an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) may be used as the therapeutic protein. In any of the methods described herein, an extracellular ActRII variant (e.g., an extracellular ActRII chimera having the sequence of any one of SEQ ID NOs: 1-43 (e.g., SEQ ID NOs: 22-43) or an extracellular ActRIIB variant having the sequence of any one of SEQ ID NOs: 269-283 (e.g., SEQ ID NOs: 270-283)) fused to an Fc domain monomer may be used as the therapeutic protein. Nucleic acids encoding the polypeptides described herein, or vectors containing said nucleic acids can also be administered according to any of the methods described herein. In any of the methods described herein, the polypeptide, nucleic acid, or vector can be administered as part of a pharmaceutical composition.

Compositions that can be administered to a subject according to the methods described herein are provided in Table 7 and Table 8, below.

### Combination Therapy

A polypeptide including an ActRII variant described herein can be administered to the subject in combination with an additional therapeutic agent. In some embodiments, the additional therapeutic agent is an angiotensin II converting enzyme inhibitor (ACE inhibitor), angiotensin II receptor blocker (ARB), beta-blocker, diuretic, angiotensin receptor-neprilysin inhibitor (ARNi, e.g., a combination of valsartan and sacubitril), calcium channel blocker, SGLT2 inhibitor (e.g., dapagliflozin and empagliflozin), ivabradine, HMG-CoA reductase inhibitor, aldosterone inhibitor (e.g., spironolactone, eplerenone, canrenone, prorenone, fineronone, and mexrenone), aliskiren, calcineurin inhibitor (e.g., cyclosporine A and tacrolimus), endothelin receptor antagonist (e.g., sitaxentan, ambrisentan, atrasentan, BQ-123, zibotentan, bosentan, macitentan, tezosentan, BQ-788, and A192621), sulodexide, vasopeptidase inhibitor (e.g., AVE7688), anti-transforming growth factor-β1 antibody, chemokine receptor 1 blocker, bone morphogenetic protein-7, PPARy agonist (e.g., rosiglitazone, pioglitazone, MRL24, Fmoc-L-Leu, SRI 664, SRI 824, GW0072, MCC555, CLX-0921, PAT5A, L-764406, nTZDpa, CDDO (2-cyano-3, 12-dioxooleana-1,9-dien-28-oic acid), ragaglitazar, an O-arylmandelic acid, and an NSAID), or matrix metalloproteinase inhibitor (e.g., BAY-12-9566). In some embodiments, the ACE inhibitor is captopril, alacepril, lisinopril, imidapril, quinapril, temocapril, delapril, benazepril, cilazapril, trandolapril, enalapril, ceronapril, fosinopril, imadapril, mobertpril, moexipril perindopril, ramipril, spirapril, or randolapril or a pharmaceutically acceptable salt thereof. In some embodiments, the ARB is losartan, candesartan, valsartan, eprosartan, telmisartan, olmesartan, azilsartan, or irbesartan. In some embodiments, the HMG-CoA reductase inhibitor is lovastatin, atorvastatin, rosuvastatin and/or rosuvastatin calcium, simvastatin, fluvastatin and/or fluvastatin sodium, pitavastatin, pravastatin, or pravastatin sodium. In some embodiments, the beta-blocker is acebutolol, atenolol, betaxolol, bisoprolol, celiprolol, metoprolol, nebivolol, propranolol, timolol, or carvedilol. In some embodiments, the diuretic is bumetanide, hydrochlorothiazide, chlortalidon, chlorothiazide, hydrochlorothiazide, xipamide, indapamide, furosemide, piretanide, torasemide, spironolactone, eplerenone, amiloride or triamterene. In some embopdiments, the calcium channel blocker is amlodipine, nifedipine, nitrendipine, nisoldipine, nicardipine, felodipine, lacidipine, lercanipidine, manidipine, isradipine, nilvadipine, verapamil, gallopamil or diltiazem.

The additional therapeutic agent may be administered at the same time (e.g., administration of all agents occurs within 15 minutes, 10 minutes, 5 minutes, 2 minutes or less) as the polypeptide including an ActRII variant described herein. The agents can also be administered simultaneously via co-formulation. The polypeptide including an ActRII variant described herein and the additional therapeutic agent can also be administered sequentially, such that the action of the two overlaps and their combined effect is such that the reduction in a symptom, or other parameter related to the disorder is greater than what would be observed with one agent or treatment delivered alone or in the absence of the other. The effect of the polypeptide including an ActRII variant described herein and the additional therapeutic agent can be partially additive, wholly additive, or greater than additive (e.g., synergistic). Sequential or substantially simultaneous administration of each of the polypeptide including an ActRII variant described herein and the additional therapeutic agent can be performed by any appropriate route including, but not limited to, oral routes, intravenous routes, intramuscular routes, local routes, and direct absorption through mucous membrane tissues. The polypeptide including an ActRII variant described herein and the additional therapeutic agent can be administered by the same route or by different routes. For example, the polypeptide including an ActRII variant described herein may be administered by subcutaneous injection while the additional therapeutic agent can be administered orally or by intravenous injection or infusion. The polypeptide including an ActRII variant described herein may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours or up to 1-7, 1-14, 1-21 or 1-30 days before or after the additional therapeutic agent. In some embodiments, the polypeptide including an ActRII variant described herein and the additional therapeutic agent are administered at different frequencies. For example, the polypeptide including an ActRII variant described herein can be administered once a week, once every two weeks, once every four weeks, once a month, once bimonthly, once every three months, once every four months, or once every six months and the additional therapeutic agent can be administered once or twice daily. In some embodiments, the polypeptide including an ActRII variant described herein and the additional therapeutic agent are administered at the same or at similar frequencies. For example, both the polypeptide including an ActRII variant described herein and the additional therapeutic agent can be administered once a week, once every two weeks, once every four weeks, once a month, once bimonthly, once every three months, once every four months, or once every six months.

TABLE 7

| Row | Composition |
|---|---|
| 1 | A polypeptide comprising an extracellular activin receptor type II |
| | (ActRII) chimera, the chimera having a sequence of any one of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 1), |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 2), |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 3), |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 4), |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 5), |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 6), |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 7), |
| | GRGEAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 8), |
| | GRGEAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 9), |
| | GRGEAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 10), |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 11), |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 12), |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 13), |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 14), |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 15), |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 16), |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 17), |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 18), |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 19), |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 20), |
| | and |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 21), |
| | wherein |
| | X₁ is D or R, |
| | X₂ is I, F, E, D, Y, S, N, Q, or T, |
| | X₃ is N or T, |
| | X₄ is A or E, |
| | X₅ is T or K, |
| | X₆ is E or K, |
| | X₇ is E or D, |
| | X₈ is N or S, and |
| | X₉ is Q, E, K, R, D, or N, optionally wherein the chimera is |
| | truncated from the N-terminus by deletion of 1, 2, 3, 4, 5, |
| | 6, 7, 8, or 9 amino acids, wherein the chimera retains the |
| | two amino acids before the first cysteine. |
| 2 | The polypeptide of row 1, wherein the chimera has the sequence of |
| | GAILGRAETRECIYYNANWELERTNQSGLERCEGEQX₁KRRHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 1). |
| 3 | The polypeptide of row 1, wherein the chimera has the sequence of |
| | GAILGRAETRECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 2). |
| 4 | The polypeptide of row 1, wherein the chimera has the sequence of |
| | GAILGRAETRECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 3). |
| 5 | The polypeptide of row 1, wherein the chimera has the sequence of |
| | GAILGRAETRECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 4). |
| 6 | The polypeptide of row 1, wherein the chimera has the sequence of |
| | GAILGRAETRECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 5). |
| 7 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 6). |
| 8 | The polypeptide of row 1, wherein the chimera has the sequence of |
| | GAILGRAETRECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 7). |
| 9 | The polypeptide of row 1, wherein the chimera has the sequence of |
| | GRGEAETRECIYYNANWELERTNQSGLERCEGEQX₁KRRHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 8). |
| 10 | The polypeptide of row 1, wherein the chimera has the sequence of |
| | GRGEAETRECIYYNANWELERTNQSGLERCEGEQX₁KRLHCFATWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 9). |
| 11 | The polypeptide of row 1, wherein the chimera has the sequence of |
| | GRGEAETRECIYYNANWELERTNQSGLERCEGEQX₁KRLHCYASWKNISGSIEIVKQGCWLDDX₂ |
| | X₃CYDRTDCVX₄X₅X₆X₇X₈PX₉VYFCCCEGNMCNEKFSYFPEMEVTQPTS (SEQ ID NO: 10). |
| 12 | The polypeptide of row 1, wherein the chimera has the sequence of |
| | GRGEAETRECIYYNANWELERTNQSGLERCEGEQX₁KRLHCYASWRNSSGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 11). |
| 13 | The polypeptide of row 1, wherein the chimera has the sequence of |
| | GRGEAETRECIYYNANWELERTNQSGLERCEGEQX₁KRLHCYASWRNSSGTIEIVKQGCWLDD |
| | X₂X₃CYDRTDCVX₄X₅X₆X₇X₈PX₉VYFCCCEGNMCNEKFSYFPEMEVTQPTS (SEQ ID NO: 12). |
| 14 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRTDCVX₄X₅X₆X₇X₈PX₉VYFCCCEGNMCNEKFSYFPEMEVTQPTS (SEQ ID NO: 13). |
| 15 | The polypeptide of row 1, wherein the chimera has the sequence of |
| | GRGEAETRECIYYNANWELERTNQSGLERCEGEQX₁KRLHCYASWRNSSGTIELVKKGCWLDD |
| | X₂X₃CYDRQECVX₄X₅X₆X₇X₈PX₉VYFCCCEGNMCNEKFSYFPEMEVTQPTS (SEQ ID NO: 14). |
| 16 | The polypeptide of row 1, wherein the chimera has the sequence of |
| | GAILGRSETQECIYYNANWELERTNQSGLERCEGEQX₁KRRHCFATWKNISGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 15). |
| 17 | The polypeptide of row 1, wherein the chimera has the sequence of |
| | GAILGRSETQECIYYNANWELERTNQSGLERCEGEQX₁KRLHCFATWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 16). |
| 18 | The polypeptide of row 1, wherein the chimera has the sequence of |
| | GAILGRSETQECIYYNANWELERTNQSGLERCEGEQX₁KRLHCYASWKNISGSIEIVKQGCWLDD |
| | X₂X₃CYDRTDCVX₄X₅X₆X₇X₈PX₉VYFCCCEGNMCNEKFSYFPEMEVTQPTS (SEQ ID NO: 17). |
| 19 | The polypeptide of row 1, wherein the chimera has the sequence of |
| | GAILGRSETQECIYYNANWELERTNQSGLERCEGEQX₁KRLHCYASWRNSSGSIEIVKQGCWLD |
| | DX₂X₃CYDRTDCVX₄X₅X₆X₇X₈PX₉VYFCCCEGNMCNEKFSYFPEMEVTQPTS (SEQ ID NO: 18). |
| 20 | The polypeptide of row 1, wherein the chimera has the sequence of |
| | GAILGRSETQECIYYNANWELERTNQSGLERCEGEQX₁KRLHCYASWRNSSGTIEIVKQGCWLD |
| | DX₂X₃CYDRTDCVX₄X₅X₆X₇X₈PX₉VYFCCCEGNMCNEKFSYFPEMEVTQPTS (SEQ ID NO: 19). |
| 21 | The polypeptide of row 1, wherein the chimera has the sequence of |
| | GAILGRSETQECIYYNANWELERTNQSGLERCEGEQX₁KRLHCYASWRNSSGTIELVKKGCWLD |
| | DX₂X₃CYDRTDCVX₄X₅X₆X₇X₈PX₉VYFCCCEGNMCNEKFSYFPEMEVTQPTS (SEQ ID NO: 20). |
| 22 | The polypeptide of row 1, wherein the chimera has the sequence of |
| | GAILGRSETQECIYYNANWELERTNQSGLERCEGEQX₁KRLHCYASWRNSSGTIELVKKGCWLD |
| | DX₂X₃CYDRQECVX₄X₅X₆X₇X₈PX₉VYFCCCEGNMCNEKFSYFPEMEVTQPTS (SEQ ID NO: 21). |
| 23 | The polypeptide of any one of rows 1-22, wherein X₁ is D. |
| 24 | The polypeptide of any one of rows 1-22, wherein X₁ is R. |
| 25 | The polypeptide of any one of rows 1-24, wherein X₂ is I. |
| 26 | The polypeptide of any one of rows 1-24, wherein X₂ is F. |
| 27 | The polypeptide of any one of rows 1-24, wherein X₂ is E. |
| 28 | The polypeptide of any one of rows 1-24, wherein X₂ is D. |
| 29 | The polypeptide of any one of rows 1-24, wherein X₂ is Y. |
| 30 | The polypeptide of any one of rows 1-24, wherein X₂ is S. |
| 31 | The polypeptide of any one of rows 1-24, wherein X₂ is N. |
| 32 | The polypeptide of any one of rows 1-24, wherein X₂ is Q. |
| 33 | The polypeptide of any one of rows 1-24, wherein X₂ is T. |
| 34 | The polypeptide of any one of rows 1-33, wherein X₃ is N. |
| 35 | The polypeptide of any one of rows 1-33, wherein X₃ is T. |
| 36 | The polypeptide of any one of rows 1-35, wherein X₄ is A. |
| 37 | The polypeptide of any one of rows 1-35, wherein X₄ is E. |
| 38 | The polypeptide of any one of rows 1-37, wherein X₅ is T. |
| 39 | The polypeptide of any one of rows 1-37, wherein X₅ is K. |
| 40 | The polypeptide of any one of rows 1-39, wherein X₆ is E. |
| 41 | The polypeptide of any one of rows 1-39, wherein X₆ is K. |
| 42 | The polypeptide of any one of rows 1-41, wherein X₇ is E. |
| 43 | The polypeptide of any one of rows 1-41, wherein X₇ is D. |
| 44 | The polypeptide of any one of rows 1-43, wherein X₈ is N. |
| 45 | The polypeptide of any one of rows 1-43, wherein X₈ is S. |
| 46 | The polypeptide of any one of rows 1-45, wherein X₉ is Q. |
| 47 | The polypeptide of any one of rows 1-45, wherein X₉ is E. |
| 48 | The polypeptide of any one of rows 1-45, wherein X₉ is K. |
| 49 | The polypeptide of any one of rows 1-45, wherein X₉ is R. |
| 50 | The polypeptide of any one of rows 1-45, wherein X₉ is D. |
| 51 | The polypeptide of any one of rows 1-45, wherein X₉ is N. |
| 52 | The polypeptide of any one of rows 1-51, wherein X₅ is T, X₆ is E, X₇ is E, and X₈ is N. |
| 53 | The polypeptide of any one of rows 1-51, wherein X₅ is T, X₆ is K, X₇ is E, and X₈ is N. |
| 54 | The polypeptide of any one of rows 1-53, wherein X₂ is E and X₃ is T. |
| 55 | The polypeptide of any one of rows 1-53, wherein X₂ is I or F and X₃ is N. |
| 56 | The polypeptide of row 55, wherein X₂ is I. |
| 57 | The polypeptide of row 55, wherein X₂ is F. |
| 58 | The polypeptide of row 1, wherein the chimera has the sequence of any one of SEQ ID NOs: 22- |
| | 43. |
| 59 | The polypeptide of row 58, wherein the chimera has the sequence of SEQ ID NO: 22. |
| 60 | The polypeptide of row 58, wherein the chimera has the sequence of SEQ ID NO: 23. |
| 61 | The polypeptide of row 58, wherein the chimera has the sequence of SEQ ID NO: 24. |
| 62 | The polypeptide of row 58, wherein the chimera has the sequence of SEQ ID NO: 25. |
| 63 | The polypeptide of row 58, wherein the chimera has the sequence of SEQ ID NO: 40. |
| 64 | The polypeptide of row 58, wherein the chimera has the sequence of SEQ ID NO: 41. |
| 65 | The polypeptide of row 58, wherein the chimera has the sequence of SEQ ID NO: 28. |
| 66 | The polypeptide of row 58, wherein the chimera has the sequence of SEQ ID NO: 42. |
| 67 | The polypeptide of row 58, wherein the chimera has the sequence of SEQ ID NO: 43. |
| 68 | The polypeptide of row 58, wherein the chimera has the sequence of SEQ ID NO: 37. |
| 69 | The polypeptide of any one of rows 1-68, wherein the chimera is truncated from the N-terminus |
| | by deletion of one amino acid. |
| 70 | The polypeptide of any one of rows 1-68, wherein the chimera is truncated from the N-terminus |
| | by deletion of two amino acids. |
| 71 | The polypeptide of any one of rows 1-68, wherein the chimera is truncated from the N-terminus |
| | by deletion of three amino acids. |
| 72 | The polypeptide of any one of rows 1-68, wherein the chimera is truncated from the N-terminus |
| | by deletion of four amino acids. |
| 73 | The polypeptide of any one of rows 1-68, wherein the chimera is truncated from the N-terminus |
| | by deletion of five amino acids. |
| 74 | The polypeptide of any one of rows 1-68, wherein the chimera is truncated from the N-terminus |
| | by deletion of six amino acids. |
| 75 | The polypeptide of any one of rows 1-68, wherein the chimera is truncated from the N-terminus |
| | by deletion of seven amino acids. |
| 76 | The polypeptide of any one of rows 1-68, wherein the chimera is truncated from the N-terminus |
| | by deletion of eight amino acids. |
| 77 | The polypeptide of any one of rows 1-68, wherein the chimera is truncated from the N-terminus |
| | by deletion of nine amino acids. |
| 78 | The polypeptide of any one of rows 1 and 69-77, wherein the chimera has the sequence of any |
| | one of SEQ ID NOs: 111-183. |
| 79 | The polypeptide of row 78, wherein the chimera has the sequence of SEQ ID NO: 111. |
| 80 | The polypeptide of row 78, wherein the chimera has the sequence of SEQ ID NO: 116. |
| 81 | The polypeptide of row 78, wherein the chimera has the sequence of SEQ ID NO: 117. |
| 82 | The polypeptide of row 78, wherein the chimera has the sequence of SEQ ID NO: 118. |
| 83 | The polypeptide of row 78, wherein the chimera has the sequence of SEQ ID NO: 119. |
| 84 | The polypeptide of row 78, wherein the chimera has the sequence of SEQ ID NO: 120. |
| 85 | The polypeptide of row 78, wherein the chimera has the sequence of SEQ ID NO: 121. |
| 86 | The polypeptide of row 78, wherein the chimera has the sequence of SEQ ID NO: 122. |
| 87 | The polypeptide of row 78, wherein the chimera has the sequence of SEQ ID NO: 126. |
| 88 | The polypeptide of row 78, wherein the chimera has the sequence of SEQ ID NO: 130. |
| 89 | The polypeptide of row 78, wherein the chimera has the sequence of SEQ ID NO: 142. |
| 90 | The polypeptide of row 78, wherein the chimera has the sequence of SEQ ID NO: 143. |
| 91 | The polypeptide of row 78, wherein the chimera has the sequence of SEQ ID NO: 146. |
| 92 | The polypeptide of row 78, wherein the chimera has the sequence of SEQ ID NO: 147. |
| 93 | The polypeptide of row 78, wherein the chimera has the sequence of SEQ ID NO: 149. |
| 94 | The polypeptide of row 78, wherein the chimera has the sequence of SEQ ID NO: 150. |
| 95 | The polypeptide of row 78, wherein the chimera has the sequence of SEQ ID NO: 151. |
| 96 | The polypeptide of row 78, wherein the chimera has the sequence of SEQ ID NO: 152. |
| 97 | The polypeptide of row 78, wherein the chimera has the sequence of SEQ ID NO: 153. |
| 98 | The polypeptide of row 78, wherein the chimera has the sequence of SEQ ID NO: 154. |
| 99 | The polypeptide of row 78, wherein the chimera has the sequence of SEQ ID NO: 155. |
| 100 | The polypeptide of row 78, wherein the chimera has the sequence of SEQ ID NO: 156. |
| 101 | The polypeptide of row 78, wherein the chimera has the sequence of SEQ ID NO: 158. |
| 102 | The polypeptide of row 78, wherein the chimera has the sequence of SEQ ID NO: 159. |
| 103 | The polypeptide of row 78, wherein the chimera has the sequence of SEQ ID NO: 161. |
| 104 | The polypeptide of row 78, wherein the chimera has the sequence of SEQ ID NO: 162. |
| 105 | The polypeptide of row 78, wherein the chimera has the sequence of SEQ ID NO: 163. |
| 106 | The polypeptide of row 78, wherein the chimera has the sequence of SEQ ID NO: 164. |
| 107 | The polypeptide of row 78, wherein the chimera has the sequence of SEQ ID NO: 167. |
| 108 | The polypeptide of row 78, wherein the chimera has the sequence of SEQ ID NO: 171. |
| 109 | The polypeptide of row 78, wherein the chimera has the sequence of SEQ ID NO: 172. |
| 110 | The polypeptide of row 78, wherein the chimera has the sequence of SEQ ID NO: 173. |
| 111 | The polypeptide of row 78, wherein the chimera has the sequence of SEQ ID NO: 176. |
| 112 | The polypeptide of row 78, wherein the chimera has the sequence of SEQ ID NO: 177. |
| 113 | The polypeptide of row 78, wherein the chimera has the sequence of SEQ ID NO: 179. |
| 114 | The polypeptide of row 78, wherein the chimera has the sequence of SEQ ID NO: 183. |
| 115 | The polypeptide of any one of rows 1-114, wherein the polypeptide (e.g., the chimera) |
| | further includes a C-terminal extension of one or more amino acids (e.g., 1, 2, 3, 4, |
| | 5, 6, or more amino acids from wild-type extracellular ActRIIA or ActRIIB). |
| 116 | The polypeptide of row 115, wherein the C-terminal extension is NP. |
| 117 | The polypeptide of row 115, wherein the C-terminal extension is NPVTPK (SEQ ID NO: 104). |
| 118 | The polypeptide of any one of rows 1-117, wherein the polypeptide further includes an |
| | Fc domain monomer fused to the C-terminus of the polypeptide (e.g., the C-terminus |
| | of the chimera) by way of a linker. |
| 119 | The polypeptide of row 118, wherein the Fc domain monomer is an IgG1 Fc domain monomer. |
| 120 | The polypeptide of row 119, wherein the IgG1 Fc domain monomer is a human IgG1 Fc domain |
| | monomer. |
| 121 | The polypeptide of row 118, wherein the Fc domain monomer has the sequence of SEQ ID NO: |
| | 48, SEQ ID NO: 100, or SEQ ID NO: 264. |
| 122 | The polypeptide of row 121, wherein the Fc domain monomer has the sequence of SEQ ID NO: |
| | 100 or SEQ ID NO: 264. |
| 123 | The polypeptide of any one of rows 118-122, wherein the polypeptide has the sequence of any |
| | one of SEQ ID NOs: 107-110 and SEQ ID NOs: 184-263. |
| 124 | The polypeptide of row 123, wherein the polypeptide has the sequence of SEQ ID NO: 107. |
| 125 | The polypeptide of row 123, wherein the polypeptide has the sequence of SEQ ID NO: 108. |
| 126 | The polypeptide of row 123, wherein the polypeptide has the sequence of SEQ ID NO: 109. |
| 127 | The polypeptide of row 123, wherein the polypeptide has the sequence of SEQ ID NO: 110. |
| 128 | The polypeptide of row 123, wherein the polypeptide has the sequence of SEQ ID NO: 184. |
| 129 | The polypeptide of row 123, wherein the polypeptide has the sequence of SEQ ID NO: 189. |
| 130 | The polypeptide of row 123, wherein the polypeptide has the sequence of SEQ ID NO: 190. |
| 131 | The polypeptide of row 123, wherein the polypeptide has the sequence of SEQ ID NO: 191. |
| 132 | The polypeptide of row 123, wherein the polypeptide has the sequence of SEQ ID NO: 192. |
| 133 | The polypeptide of row 123, wherein the polypeptide has the sequence of SEQ ID NO: 193. |
| 134 | The polypeptide of row 123, wherein the polypeptide has the sequence of SEQ ID NO: 194. |
| 135 | The polypeptide of row 123, wherein the polypeptide has the sequence of SEQ ID NO: 195. |
| 136 | The polypeptide of row 123, wherein the polypeptide has the sequence of SEQ ID NO: 199. |
| 137 | The polypeptide of row 123, wherein the polypeptide has the sequence of SEQ ID NO: 200. |
| 138 | The polypeptide of row 123, wherein the polypeptide has the sequence of SEQ ID NO: 213. |
| 139 | The polypeptide of row 123, wherein the polypeptide has the sequence of SEQ ID NO: 214. |
| 140 | The polypeptide of row 123, wherein the polypeptide has the sequence of SEQ ID NO: 215. |
| 141 | The polypeptide of row 123, wherein the polypeptide has the sequence of SEQ ID NO: 216. |
| 142 | The polypeptide of row 123, wherein the polypeptide has the sequence of SEQ ID NO: 217. |
| 143 | The polypeptide of row 123, wherein the polypeptide has the sequence of SEQ ID NO: 218. |
| 144 | The polypeptide of row 123, wherein the polypeptide has the sequence of SEQ ID NO: 221. |
| 145 | The polypeptide of row 123, wherein the polypeptide has the sequence of SEQ ID NO: 222. |
| 146 | The polypeptide of row 123, wherein the polypeptide has the sequence of SEQ ID NO: 225. |
| 147 | The polypeptide of row 123, wherein the polypeptide has the sequence of SEQ ID NO: 226. |
| 148 | The polypeptide of row 123, wherein the polypeptide has the sequence of SEQ ID NO: 228. |
| 149 | The polypeptide of row 123, wherein the polypeptide has the sequence of SEQ ID NO: 229. |
| 150 | The polypeptide of row 123, wherein the polypeptide has the sequence of SEQ ID NO: 230. |
| 151 | The polypeptide of row 123, wherein the polypeptide has the sequence of SEQ ID NO: 231. |
| 152 | The polypeptide of row 123, wherein the polypeptide has the sequence of SEQ ID NO: 232. |
| 153 | The polypeptide of row 123, wherein the polypeptide has the sequence of SEQ ID NO: 233. |
| 154 | The polypeptide of row 123, wherein the polypeptide has the sequence of SEQ ID NO: 234. |
| 155 | The polypeptide of row 123, wherein the polypeptide has the sequence of SEQ ID NO: 235. |
| 156 | The polypeptide of row 123, wherein the polypeptide has the sequence of SEQ ID NO: 236. |
| 157 | The polypeptide of row 123, wherein the polypeptide has the sequence of SEQ ID NO: 237. |
| 158 | The polypeptide of row 123, wherein the polypeptide has the sequence of SEQ ID NO: 238. |
| 159 | The polypeptide of row 123, wherein the polypeptide has the sequence of SEQ ID NO: 240. |
| 160 | The polypeptide of row 123, wherein the polypeptide has the sequence of SEQ ID NO: 243. |
| 161 | The polypeptide of row 123, wherein the polypeptide has the sequence of SEQ ID NO: 246. |
| 162 | The polypeptide of row 123, wherein the polypeptide has the sequence of SEQ ID NO: 248. |
| 163 | The polypeptide of row 123, wherein the polypeptide has the sequence of SEQ ID NO: 251. |
| 164 | The polypeptide of row 123, wherein the polypeptide has the sequence of SEQ ID NO: 253. |
| 165 | The polypeptide of row 123, wherein the polypeptide has the sequence of SEQ ID NO: 257. |
| 166 | The polypeptide of row 123, wherein the polypeptide has the sequence of SEQ ID NO: 259. |
| 167 | The polypeptide of row 123, wherein the polypeptide has the sequence of SEQ ID NO: 263. |
| 168 | The polypeptide of any one of rows 118-167, wherein the polypeptide forms a dimer. |
| 169 | The polypeptide of row 168, wherein the polypeptide forms a homodimer. |
| 170 | The polypeptide of any one of rows 118-169, wherein the linker is an amino acid spacer. |
| 171 | The polypeptide of row 170, wherein the amino acid spacer is GGG, GGGA (SEQ ID NO: 49), |
| | GGGG (SEQ ID NO: 51), GGGAG (SEQ ID NO: 81), GGGAGG (SEQ ID NO: 82), or GGGAGGG |
| | (SEQ ID NO: 83). |
| 172 | The polypeptide of row 170, wherein the amino acid spacer is GGS, GGGS (SEQ ID NO: 50), |
| | GGGGS (SEQ ID NO: 53), GGSG (SEQ ID NO: 56), or SGGG (SEQ ID NO: 58). |
| 173 | The polypeptide of row 170, wherein the amino acid spacer is GA, GS, GG, GGA, GGS, GGGS |
| | (SEQ ID NO: 50), GGGGA (SEQ ID NO: 52), GGGGS (SEQ ID NO: 53), GGGGG (SEQ ID NO: |
| | 54), GGAG (SEQ ID NO: 55), GGSG (SEQ ID NO: 56), AGGG (SEQ ID NO: 57), SGGG (SEQ ID |
| | NO: 58), GAGA (SEQ ID NO: 59), GSGS (SEQ ID NO: 60), GAGAGA (SEQ ID NO: 61), |
| | GSGSGS (SEQ ID NO: 62), GAGAGAGA (SEQ ID NO: 63), GSGSGSGS (SEQ ID NO: 64), |
| | GAGAGAGAGA (SEQ ID NO: 65), GSGSGSGSGS (SEQ ID NO: 66), GAGAGAGAGAGA (SEQ |
| | ID NO: 67), GSGSGSGSGSGS (SEQ ID NO: 68), GGAGGA (SEQ ID NO: 69), GGSGGS (SEQ |
| | ID NO: 70), GGAGGAGGA (SEQ ID NO: 71), GGSGGSGGS (SEQ ID NO: 72), |
| | GGAGGAGGAGGA (SEQ ID NO: 73), GGSGGSGGSGGS (SEQ ID NO: 74), GGAGGGAG (SEQ |
| | ID NO: 75), GGSGGGSG (SEQ ID NO: 76), GGAGGGAGGGAG (SEQ ID NO: 77), |
| | GGSGGGSGGGSG (SEQ ID NO: 78), GGGGAGGGGAGGGGA (SEQ ID NO: 79), |
| | GGGGSGGGGSGGGGS (SEQ ID NO: 80), AAAL (SEQ ID NO: 84), AAAK (SEQ ID NO: 85), |
| | AAAR (SEQ ID NO: 86), EGKSSGSGSESKST (SEQ ID NO: 87), GSAGSAAGSGEF (SEQ ID |
| | NO: 88), AEAAAKEAAAKA (SEQ ID NO: 89), KESGSVSSEQLAQFRSLD (SEQ ID NO: 90), |
| | GENLYFQSGG (SEQ ID NO: 91), SACYCELS (SEQ ID NO: 92), RSIAT (SEQ ID NO: 93), |
| | RPACKIPNDLKQKVMNH (SEQ ID NO: 94), |
| | GGSAGGSGSGSSGGSSGASGTGTAGGTGSGSGTGSG (SEQ ID NO: 95), |
| | AAANSSIDLISVPVDSR (SEQ ID NO: 96), |
| | GGSGGGSEGGGSEGGGSEGGGSEGGGSEGGGSGGGS (SEQ ID NO: 97), EAAAK (SEQ ID |
| | NO: 98), or PAPAP(SEQ ID NO: 99). |
| 174 | The polypeptide of any one of rows 1-173, wherein the polypeptide (e.g., an ActRII chimera-Fc |
| | fusion protein) has a serum half-life of at least seven days. |
| 175 | The polypeptide of any one of rows 1-174, wherein the polypeptide has increased binding to one |
| | or more ActRII ligands (e.g., activin A, activin B, myostatin, and/or GDF-11) compared to wild- |
| | type ActRIIA and/or wild-type ActRIIB (e.g., wild-type extracellular ActRIIA and/or ActRIIB). |
| 176 | The polypeptide of any one of rows 1-175, wherein the polypeptide has decreased binding to |
| | bone morphogenetic protein 9 (BMP9, e.g., human BMP9) compared to wild-type ActRIIB (e.g., |
| | wild-type extracellular ActRIIB). |
| 177 | The polypeptide of any one of rows 1-176, wherein the polypeptide binds to activin A, |
| | activin B,and/or myostatin and has reduced or weak binding to human BMP9 |
| | (e.g., compared to wild-type extracellular ActRIIB). |
| 178 | The polypeptide of any one of rows 1-177, wherein the polypeptide does not substantially |
| | bind to human BMP9. |
| 179 | The polypeptide of any one of rows 1-178, wherein the polypeptide binds to human activin |
| | A with a KD of 800 pM or less. |
| 180 | The polypeptide of any one of rows 1-179, wherein the polypeptide binds to human activin B |
| | with a KD of 800 pM or less. |
| 181 | The polypeptide of any one of rows 1-180, wherein the polypeptide binds to human GDF-11 with |
| | a KD of 5 pM or higher. |
| 182 | A nucleic acid molecule encoding the polypeptide of any one of rows 1-181. |
| 183 | A vector comprising the nucleic acid molecule of row 182. |
| 184 | A host cell that expresses the polypeptide of any one of rows 1-181, wherein the host cell |
| | comprises the nucleic acid molecule of row 182 or the vector of row 183, wherein the nucleic |
| | acid molecule or vector is expressed in the host cell. |
| 185 | A pharmaceutical composition comprising the polypeptide of any one of rows 1-181, the nucleic |
| | acid molecule of row 182, or the vector of row 183 and one or more pharmaceutically acceptable |
| | carriers or excipients. |
| 186 | The pharmaceutical composition of row 185, wherein the polypeptide, nucleic acid molecule, |
| | or vector is in a therapeutically effective amount. |
| 187 | A construct comprising two identical polypeptides (e.g., a homodimer), each |
| | comprising an extracellular ActRII chimera of any one of rows 1-117 (e.g., |
| | an ActRII chimera having a sequence of any one of SEQ ID NOs: 1-43 and 111-183) |
| | fused (e.g., linked using an amino acid spacer) to the N- or C-terminus of an |
| | Fc domain monomer (e.g., the sequence of SEQ ID NO: 48, SEQ ID NO: 100, or |
| | SEQ ID NO: 264). The two Fc domain monomers in the two polypeptides interact |
| | to form an Fc domain in the construct. |
| 188 | A construct comprising two different polypeptides (e.g., a heterodimer), each |
| | comprising an extracellular ActRII chimera of any one of rows 1-117 (e.g., an |
| | ActRII chimera having a sequence of any one of SEQ ID NOs: 1-43 and 111-183) |
| | fused (e.g., linked using an amino acid spacer) to the N- or C-terminus of an |
| | Fc domain monomer (e.g., the sequence of SEQ ID NO: 48, SEQ ID NO: 100, or SEQ |
| | ID NO: 264). The two Fc domain monomers in the two polypeptides interact to form |
| | an Fc domain in the construct. |

TABLE 8

| Row | Composition |
|---|---|
| 1 | A polypeptide comprising an extracellular activin receptor type IIB (ActRIIB) variant, the |
| | variant having one or more amino acid substitutions relative to the sequence of |
| | GRGEAETRECIYYNANWELERTNQSGLERCEGEQDKRLHCYASWRNSSGTIELVKKGCWL |
| | DDFNCYDRQECVATEENPQVYFCCCEGNFCNERFTHLPEAGGPEVTYEPPPTAPT (SEQ ID |
| | NO: 45), wherein the variant comprises one or more amino acid substitutions that impart |
| | reduced BMP9 binding relative to wild type extracellular ActRIIB and one or more additional |
| | amino acid substitutions, wherein the substitutions that reduce BMP9 binding comprise one or |
| | more of: |
| | a) amino acid substitution E75K; |
| | b) amino acid substitutions Q69T and E70D; or |
| | c) amino acid substitutions Q69D and E70T, |
| | optionally wherein the variant is truncated from the N-terminus by deletion |
| | of 1, 2, 3, 4, 5, 6, or 7 amino acids. |
| 2 | The polypeptide of row 1, wherein the variant comprises one or more amino acid substitutions |
| | selected from the group consisting of 111L, Y12F, L19K, E20D, S25T, L27V, R29P, E31Y, |
| | E33D, Q34K, L38R, Y41F, R45K, S471, S48T, T50S, 151L, L53I, K56Q, F63I, T74K, E76D, |
| | N77S, Q79E, and F89M. |
| 3 | The polypeptide of row 1 or 2, wherein the variant comprises amino acid substitutions E75K, |
| | E20D, and F631. |
| 4 | The polypeptide of row 1 or 2, wherein the variant comprises amino acid substitution E75K. |
| 5 | The polypeptide of row 4, wherein the variant comprises amino acid substitutions T74K, E76D, |
| | N77S, and Q79E. |
| 6 | The polypeptide of row 5, wherein the variant further comprises one or more additional amino |
| | acid substitutions. |
| 7 | The polypeptide of row 6, wherein the variant comprises amino acid substitutions Y41F, R45K, |
| | and K56Q. |
| 8 | The polypeptide of row 7, wherein the variant further comprises amino acid substitutions |
| | Y12F, L19K, E20D, R29P, E31Y, E33D, L38R, and F631. |
| 9 | The polypeptide of row 6, wherein the variant comprises amino acid substitutions S25T and |
| | S471. |
| 10 | The polypeptide of row 9, wherein the variant comprises amino acid substitution S48T. |
| 11 | The polypeptide of row 6, wherein the variant comprises amino acid substitution R29P. |
| 12 | The polypeptide of row 6, wherein the variant comprises amino acid substitutions E31Y, |
| | E33D, and Q34K. |
| 13 | The polypeptide of row 6, wherein the variant comprises amino acid substitutions Y12F, L19K, |
| | and E20D. |
| 14 | The polypeptide of row 6, wherein the variant comprises amino acid substitutions E31Y, |
| | E33D, and L38R. |
| 15 | The polypeptide of row 1 or 2, wherein the variant comprises amino acid substitutions Q69T |
| | and E70D. |
| 16 | The polypeptide of any one of rows 1, 2, and 15, wherein the variant comprises amino acid |
| | substitutions Q69T and E70D and additional amino acid substitutions 111L, L27V, Q34K, |
| | T50S, 151L, L531, and F89M. |
| 17 | The polypeptide of row 1 or 2, wherein the variant comprises amino acid substitutions Q69D |
| | and E70T. |
| 18 | The polypeptide of any one of rows 1, 2, and 17, wherein the variant comprises amino acid |
| | substitutions Q69D and E70T and additional amino acid substitutions 111L, L27V, Q34K, |
| | T50S, 151L, L531, and F89M. |
| 19 | The polypeptide of any one of rows 15-18, wherein the variant comprises amino acid |
| | substitution E75K. |
| 20 | A polypeptide comprising an ActRIIB variant, the variant having a sequence of |
| | GRGEAETRECX₁X₂YNANWEX₃X₄RTNQX₅GX₆EX₇CX₈GX₉X₁₀DKRX₁₁HCX₁₂ASWX₁₃NX₁₄X₁₅ |
| | GX₁₆X₁₇EX₁₈VKX₁₉GCWLDDX₂₀NCYDRX₂₁X₂₂CVAX₂₃X₂₄X₂₅X₂₆PX₂₇VYFCCCEGNX₂₈CNERF |
| | THLPEAGGPEVTYEPPPTAPT (SEQ ID NO: 269), |
| | wherein |
| | X₁ is I or L; |
| | X₂ is F or Y; |
| | X₃ is L or K; |
| | X₄ is D or E; |
| | X₅ is T or S; |
| | X₆ is L or V; |
| | X₇ is P or R; |
| | X₈ is Y or E; |
| | X₉ is D or E; |
| | X₁₀ is K or Q; |
| | X₁₁ is R or L; |
| | X₁₂ is Y or F; |
| | X₁₃ is R or K; |
| | X₁₄ is S or I; |
| | X₁₅ is S or T; |
| | X₁₆ is S or T; |
| | X₁₇ is I or L; |
| | X₁₈ is I or L; |
| | X₁₉ is K or Q; |
| | X₂₀ is F or I; |
| | X₂₁ is Q, T, or D; |
| | X₂₂ is E, D, or T; |
| | X₂₃ is K or T; |
| | X₂₄ is K or E; |
| | X₂₅ is D or E; |
| | X₂₆ is S or N; |
| | X₂₇ is E or Q; and |
| | X₂₈ is F or M, and wherein |
| | X₂₄ is E and/or either |
| | X₂₁ is T and |
| | X₂₂ is D or |
| | X₂₁ is D and |
| | X₂₂ is T, and wherein the variant has at least one amino |
| | acid substitution relative to a wild-type extracellular |
| | ActRIIB having the sequence of SEQ ID NO: 45, optionally |
| | wherein the variant is truncated from the N-terminus by |
| | deletion of 1, 2, 3, 4, 5, 6, or 7 amino acids. |
| 21 | The polypeptide of row 20, wherein X₁ is I. |
| 22 | The polypeptide of row 20, wherein X₁ is L. |
| 23 | The polypeptide of any one of rows 20-22, wherein X₂ is F. |
| 24 | The polypeptide of any one of rows 20-22, wherein X₂ is Y. |
| 25 | The polypeptide of any one of rows 20-24, wherein X₃ is L. |
| 26 | The polypeptide of any one of rows 20-24, wherein X₃ is K. |
| 27 | The polypeptide of any one of rows 20-26, wherein X₄ is D. |
| 28 | The polypeptide of any one of rows 20-26, wherein X₄ is E. |
| 29 | The polypeptide of any one of rows 20-28, wherein X₅ is T. |
| 30 | The polypeptide of any one of rows 20-28, wherein X₅ is S. |
| 31 | The polypeptide of any one of rows 20-30, wherein X₆ is L. |
| 32 | The polypeptide of any one of rows 20-30, wherein X₆ is V. |
| 33 | The polypeptide of any one of rows 20-32, wherein X₇ is P. |
| 34 | The polypeptide of any one of rows 20-32, wherein X₇ is R. |
| 35 | The polypeptide of any one of rows 20-34, wherein X₈ is Y. |
| 36 | The polypeptide of any one of rows 20-34, wherein X₈ is E. |
| 37 | The polypeptide of any one of rows 20-36, wherein X₉ is D. |
| 38 | The polypeptide of any one of rows 20-36, wherein X₉ is E. |
| 39 | The polypeptide of any one of rows 20-38, wherein X₁₀ is K. |
| 40 | The polypeptide of any one of rows 20-38, wherein X₁₀ is Q. |
| 41 | The polypeptide of any one of rows 20-40, wherein X₁₁ is R. |
| 42 | The polypeptide of any one of rows 20-40, wherein X₁₁ is L. |
| 43 | The polypeptide of any one of rows 20-42, wherein X₁₂ is Y. |
| 44 | The polypeptide of any one of rows 20-42, wherein X₁₂ is F. |
| 45 | The polypeptide of any one of rows 20-44, wherein X₁₃ is R. |
| 46 | The polypeptide of any one of rows 20-44, wherein X₁₃ is K. |
| 47 | The polypeptide of any one of rows 20-46, wherein X₁₄ is S. |
| 48 | The polypeptide of any one of rows 20-46, wherein X₁₄ is I. |
| 49 | The polypeptide of any one of rows 20-48, wherein X₁₅ is S. |
| 50 | The polypeptide of any one of rows 20-48, wherein X₁₅ is T. |
| 51 | The polypeptide of any one of rows 20-50, wherein X₁₆ is S. |
| 52 | The polypeptide of any one of rows 20-50, wherein X₁₆ is T. |
| 53 | The polypeptide of any one of rows 20-52, wherein X₁₇ is I. |
| 54 | The polypeptide of any one of rows 20-52, wherein X₁₇ is L. |
| 55 | The polypeptide of any one of rows 20-54, wherein X₁₈ is I. |
| 56 | The polypeptide of any one of rows 20-54, wherein X₁₈ is L. |
| 57 | The polypeptide of any one of rows 20-56, wherein X₁₉ is K. |
| 58 | The polypeptide of any one of rows 20-56, wherein X₁₉ is Q. |
| 59 | The polypeptide of any one of rows 20-58, wherein X₂₀ is F. |
| 60 | The polypeptide of any one of rows 20-58, wherein X₂₀ is I. |
| 61 | The polypeptide of any one of rows 20-60, wherein X₂₁ is Q. |
| 62 | The polypeptide of any one of rows 20-60, wherein X₂₁ is T. |
| 63 | The polypeptide of any one of rows 20-60, wherein X₂₁ is D. |
| 64 | The polypeptide of any one of rows 20-61, wherein X₂₂ is E. |
| 65 | The polypeptide of any one of rows 20-60 and 62, wherein X₂₂ is D. |
| 66 | The polypeptide of any one of rows 20-60 and 63, wherein X₂₂ is T. |
| 67 | The polypeptide of any one of rows 20-66, wherein X₂₃ is K. |
| 68 | The polypeptide of any one of rows 20-66, wherein X₂₃ is T. |
| 69 | The polypeptide of any one of rows 20-68, wherein X₂₄ is K. |
| 70 | The polypeptide of any one of rows 20-60, 62, 63, and 65-68, wherein X₂₄ is E. |
| 71 | The polypeptide of any one of rows 20-70, wherein X₂₅ is D. |
| 72 | The polypeptide of any one of rows 20-70, wherein X₂₅ is E. |
| 73 | The polypeptide of any one of rows 20-72, wherein X₂₆ is S. |
| 74 | The polypeptide of any one of rows 20-72, wherein X₂₆ is N. |
| 75 | The polypeptide of any one of rows 20-74, wherein X₂₇ is E. |
| 76 | The polypeptide of any one of rows 20-74, wherein X₂₇ is Q. |
| 77 | The polypeptide of any one of rows 20-76, wherein X₂₈ is F. |
| 78 | The polypeptide of any one of rows 20-76, wherein X₂₈ is M. |
| 79 | The polypeptide of any one of rows 20-78, wherein X₂₃ is T, X₂₄ is K, X₂₅ is E, and X₂₆ is N. |
| 80 | The polypeptide of any one of rows 20-78, wherein X₂₃ is T, X₂₄ is E, X₂₅ is E, and X₂₆ is N. |
| 81 | The polypeptide of any one of rows 20-78, wherein X₂₃ is K, X₂₄ is K, X₂₅ is D, and X₂₆ is S. |
| 82 | The polypeptide of any one of rows 1-81, wherein the variant has the sequence of any one of |
| | SEQ ID NOs: 270-283. |
| 83 | The polypeptide of row 82, wherein the variant has the sequence of SEQ ID NO: 271. |
| 84 | The polypeptide of row 82, wherein the variant has the sequence of SEQ ID NO: 277. |
| 85 | The polypeptide of row 82, wherein the variant has the sequence of SEQ ID NO: 280. |
| 86 | The polypeptide of row 82, wherein the variant has the sequence of SEQ ID NO: 281. |
| 87 | The polypeptide of row 82, wherein the variant has the sequence of SEQ ID NO: 282. |
| 88 | The polypeptide of row 82, wherein the variant has the sequence of SEQ ID NO: 283. |
| 89 | The polypeptide of any one of rows 1-88, wherein the amino acid at position X₂₄ is replaced |
| | with the amino acid K. |
| 90 | The polypeptide of any one of rows 1-88, wherein the amino acid at position X₂₄ is replaced |
| | with the amino acid E. |
| 91 | The polypeptide of any one of rows 1-90, wherein the variant is truncated from the N-terminus |
| | by deletion of one amino acid. |
| 92 | The polypeptide of any one of rows 1-90, wherein the variant is truncated from the N-terminus |
| | by deletion of two amino acids. |
| 93 | The polypeptide of any one of rows 1-90, wherein the variant is truncated from the N-terminus |
| | by deletion of three amino acids. |
| 94 | The polypeptide of any one of rows 1-90, wherein the variant is truncated from the N-terminus |
| | by deletion of four amino acids. |
| 95 | The polypeptide of any one of rows 1-90, wherein the variant is truncated from the N-terminus |
| | by deletion of five amino acids. |
| 96 | The polypeptide of any one of rows 1-90, wherein the variant is truncated from the N-terminus |
| | by deletion of six amino acids. |
| 97 | The polypeptide of any one of rows 1-90, wherein the variant is truncated from the N-terminus |
| | by deletion of seven amino acids. |
| 98 | The polypeptide of any one of rows 1-97, further comprising an Fc domain monomer fused to |
| | the C-terminus of the polypeptide (e.g., the C-terminus of the ActRIIB variant) by way of a |
| | linker. |
| 99 | The polypeptide of row 98, wherein the Fc domain monomer is an IgG1 Fc domain monomer. |
| 100 | The polypeptide of row 99, wherein the IgG1 Fc domain monomer is a human IgG1 Fc domain |
| | monomer. |
| 101 | The polypeptide of row 98, wherein the Fc domain monomer comprises the sequence of SEQ |
| | ID NO: 48, SEQ ID NO: 100, or SEQ ID NO: 264. |
| 102 | The polypeptide of row 101, wherein the Fc domain monomer comprises the sequence of |
| | SEQ ID NO: 100 or SEQ ID NO: 264. |
| 103 | The polypeptide of row 102, wherein the Fc domain monomer comprises the sequence of |
| | SEQ ID NO: 100. |
| 104 | The polypeptide of row 102, wherein the Fc domain monomer comprises the sequence of |
| | SEQ ID NO: 264. |
| 105 | The polypeptide of any one of rows 98-104, wherein the polypeptide forms a dimer. |
| 106 | The polypeptide of row 105, wherein the polypeptide forms a homodimer. |
| 107 | The polypeptide of any one of rows 98-106, wherein the linker is an amino acid spacer. |
| 108 | The polypeptide of row 107, wherein the amino acid spacer is GGG, GGGA (SEQ ID NO: 49), |
| | GGGG (SEQ ID NO: 51), GGGAG (SEQ ID NO: 81), GGGAGG (SEQ ID NO: 82), or |
| | GGGAGGG (SEQ ID NO: 83). |
| 109 | The polypeptide of row 108, wherein the amino acid spacer is GGG. |
| 110 | The polypeptide of row 103 or 109, wherein the polypeptide has the sequence of SEQ ID NO: |
| | 284. |
| 111 | The polypeptide of row 107, wherein the amino acid spacer is GA, GS, GG, GGA, GGS, GGG, |
| | GGGS (SEQ ID NO: 50), GGGGA (SEQ ID NO: 52), GGGGS (SEQ ID NO: 53), GGGGG |
| | (SEQ ID NO: 54), GGAG (SEQ ID NO: 55), GGSG (SEQ ID NO: 56), AGGG (SEQ ID NO: |
| | 57), SGGG (SEQ ID NO: 58), GAGA (SEQ ID NO: 59), GSGS (SEQ ID NO: 60), GAGAGA |
| | (SEQ ID NO: 61), GSGSGS (SEQ ID NO: 62), GAGAGAGA (SEQ ID NO: 63), GSGSGSGS |
| | (SEQ ID NO: 64), GAGAGAGAGA (SEQ ID NO: 65), GSGSGSGSGS (SEQ ID NO: 66), |
| | GAGAGAGAGAGA (SEQ ID NO: 67), GSGSGSGSGSGS (SEQ ID NO: 68), GGAGGA (SEQ |
| | ID NO: 69), GGSGGS (SEQ ID NO: 70), GGAGGAGGA (SEQ ID NO: 71), GGSGGSGGS |
| | (SEQ ID NO: 72), GGAGGAGGAGGA (SEQ ID NO: 73), GGSGGSGGSGGS (SEQ ID NO: |
| | 74), GGAGGGAG (SEQ ID NO: 75), GGSGGGSG (SEQ ID NO: 76), GGAGGGAGGGAG |
| | (SEQ ID NO: 77), GGSGGGSGGGSG (SEQ ID NO: 78), GGGGAGGGGAGGGGA (SEQ ID |
| | NO: 79), GGGGSGGGGSGGGGS (SEQ ID NO: 80), AAAL (SEQ ID NO: 84), AAAK (SEQ ID |
| | NO: 85), AAAR (SEQ ID NO: 86), EGKSSGSGSESKST (SEQ ID NO: 87), GSAGSAAGSGEF |
| | (SEQ ID NO: 88), AEAAAKEAAAKA (SEQ ID NO: 89), KESGSVSSEQLAQFRSLD (SEQ ID |
| | NO: 90), GENLYFQSGG (SEQ ID NO: 91), SACYCELS (SEQ ID NO: 92), RSIAT (SEQ ID |
| | NO: 93), RPACKIPNDLKQKVMNH (SEQ ID NO: 94), |
| | GGSAGGSGSGSSGGSSGASGTGTAGGTGSGSGTGSG (SEQ ID NO: 95), |
| | AAANSSIDLISVPVDSR (SEQ ID NO: 96), |
| | GGSGGGSEGGGSEGGGSEGGGSEGGGSEGGGSGGGS (SEQ ID NO: 97), EAAAK (SEQ |
| | ID NO: 98), or PAPAP(SEQ ID NO: 99). |
| 112 | The polypeptide of any one of rows 1-111, wherein the polypeptide has a serum |
| | half-life of atleast 7 days. |
| 113 | The polypeptide of any one of rows 1-112, wherein the polypeptide binds to activin A, |
| | activin B, and/or myostatin and has reduced or weak binding to human BMP9. |
| 114 | The polypeptide of row 113, wherein the polypeptide does not substantially bind to human |
| | BMP9. |
| 115 | The polypeptide of any one of rows 1-114, wherein the polypeptide binds to human activin A |
| | with a KD of 800 pM or less. |
| 116 | The polypeptide of any one of rows 1-115, wherein the polypeptide binds to human activin B |
| | with a Kp of 800 pM or less. |
| 117 | The polypeptide of any one of rows 1-116, wherein the polypeptide binds to human GDF-11 |
| | with a Kp of 5 pM or higher. |
| 118 | A nucleic acid molecule encoding a polypeptide of any one of rows 1-117. |
| 119 | A vector comprising the nucleic acid molecule of row 118. |
| 120 | A host cell that expresses a polypeptide of any one of rows 1-117, wherein the host cell |
| | comprises a nucleic acid molecule of row 118 or a vector of row 119, wherein the nucleic |
| | acid molecule or vector is expressed in the host cell. |
| 121 | A pharmaceutical composition comprising a polypeptide of any one of rows 1-117, a nucleic |
| | acid molecule of row 118, or a vector of row 119, and one or more pharmaceutically |
| | acceptable carriers or excipients. |
| 122 | The pharmaceutical composition of row 121, wherein the polypeptide is in a therapeutically |
| | effective amount. |
| 123 | A construct comprising two identical polypeptides (e.g., a homodimer), each comprising an |
| | extracellular ActRIIB variant of any one of rows 1-97 (e.g., an ActRIIB variant having a |
| | sequence of any one of SEQ ID NOs: 269-283) fused (e.g., linked using an amino acid |
| | spacer) to the N- or C-terminus of an Fc domain monomer (e.g., the sequence of SEQ ID NO: |
| | 48, SEQ ID NO: 100, or SEQ ID NO: 264). The two Fc domain monomers in the two |
| | polypeptides interact to form an Fc domain in the construct. |
| 124 | A construct comprising two different polypeptides (e.g., a heterodimer), each comprising an |
| | extracellular ActRIIB variant of any one of rows 1-97 (e.g., an ActRIIB variant having a |
| | sequence of any one of SEQ ID NOs: 269-283) fused (e.g., linked using an amino acid |
| | spacer) to the N- or C-terminus of an Fc domain monomer (e.g., the sequence of SEQ ID NO: |
| | 48, SEQ ID NO: 100, or SEQ ID NO: 264). The two Fc domain monomers in the two |
| | polypeptides interact to form an Fc domain in the construct. |

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1—Identification of Altered Serum Biomarkers in Human Subjects Treated with ActRIIB 2.12-Fc

Healthy postmenopausal women were enrolled in a randomized, double-blind, placebo-controlled, two-part study to assess the safety, tolerability, and pharmacokinetics of ActRIIB 2.12-Fc (a homodimer of the polypeptide of SEQ ID NO: 284). Inclusion criteria included being between 45 and 70 years of age, serum FSH>40 IU/L, and a BMI>18.5 kg/m² to <32.0 kg/m², and exclusion criteria included a history of or past treatment for osteoporosis and systemic hormone replacement therapy within three months of the study. In addition to safety, tolerability, and pharmacokinetics, serum biomarkers were also assessed. Described below are results from analysis of serum samples from healthy postmenopausal women administered one subcutaneous dose of either ActRIIB 2.12-Fc at a dose of 4.5 mg/kg or placebo.

Blood samples were collected in SST™ II advance (gold cap, BD Cat #367956) vacutainers, mixed by inverting six times, and placed upright at ambient temperature for a minimum of 30 minutes to a maximum of 60 minutes to allow for clot formation. Samples were centrifuged at 20° C. for 10 mins at 1,300-2999 g (RCF). The supernatant serum was aspirated, aliquoted into microtubes, and stored at −80° C. until used for analysis.

Serum biomarkers were assessed in participants who received a placebo (n=5) on day 1 (prior to treatment administration, considered baseline) and day 15, and in participants who received ActRIIB 2.12-Fc at 4.5 mg/kg (n=6) on day 1 (prior to treatment administration, considered baseline), day 7, day 15, day 22, and day 29.

All samples were measured with the SOMASCAN® platform (SOMASCAN® Proteomics) (www.somalogic.com) (SOMALOGIC®, Boulder, CO) containing 7,596 aptamers (6,408 human protein analytes) that provides measurements of the relative binding of the serum sample to each of the aptamers in relative fluorescence units (RFU). Calibration and normalization samples were used following the manufacturer's recommended protocol. Data standardization was performed according to the SOMASCAN® platform data quality-control protocols. To standardize SOMASCAN® assay results, raw SOMASCAN® assay data were first normalized to remove hybridization variation within a run (hybridization normalization), followed by median signal normalization across all samples to remove other assay biases within the run.

### SOMASCAN® Data Analysis

Multiplex assay: Fifty-five samples were submitted to SomaLogic, LLC, to measure 6,408 proteins by the SOMASCAN® assay. For this analysis, SOMAMER® reagents (aptamers) labeled with a photocleavable linker and biotin were immobilized on streptavidin-coated beads. Each serum sample was incubated with reagents, and serum proteins were allowed to bind, forming SOMAMER®-target protein complexes. Unbound proteins were washed away, and bound proteins were photocleaved with UV light, leaving only the reagents representing the once-bound proteins. The reagents were then bound to complementary sequences of DNA hybridization probes on a microarray. Probes were then quantified by fluorescence. The result was measured as relative fluorescent units (RFU) and is directly proportional to the amount of target protein in the original serum sample.

The SOMASCAN® assay results were normalized to correct systematic effects introduced during the DNA hybridization step. A control sequence introduced into the assay before hybridization was used to calculate a scaling factor, and each sample was normalized to this factor. Additionally, median normalization allows for comparing signals across a plate by correcting for introduced variation from the assay or natural variation in samples' total protein concentrations. Finally, the median signal intensity from each subarray was used to calculate a sample-based scaling factor.

### Statistical Analysis.

Quality control assessment and exploratory data analysis were performed for all samples in this study. Hybridization control normalization, intraplate median signal normalization, median signal normalization to a human serum reference, and plate scaling and calibration were performed by SomaLogic for this data. All samples passed the SomaLogic initial quality control assessments for normalization and calibration.

The R package “readat” (Cotton et al., 2016, BMC Bioinformatics) was used to read the SOMASCAN® intensity data. Quality control assessment of proteome profiles was performed using four automated outlier tests, in addition to manual inspection of MA and density plots. No samples failed other automated outlier tests.

Baseline normalization was conducted by combining placebo day 1 and day 15 samples, and ActRIIB 2.12-Fc day 1 samples to generate a baseline group. We then compared this baseline group to either ActRIIB 2.12-Fc day 7, day 15, day 22, and day 29 combined. Models were fit in R with the limma package and log FC extracted for the placebo versus drug comparison.

### Results

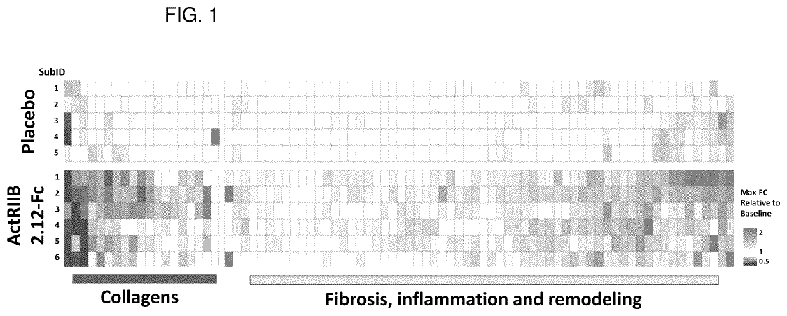

Of the 6,408 proteins in the SOMASCAN® panel, 81 proteins were significantly differentially expressed from baseline in post-menopausal women treated with ActRIIB 2.12-Fc at 4.5 mg/kg versus placebo, with an FDR threshold of 0.01. Of these 81 proteins, 63 were upregulated, and 18 were downregulated vs placebo (FIG. 1).

A clustering analysis was performed on the 81 differentially expressed serum proteins, which found that these proteins have known functions related to inflammation and extracellular matrix remodeling pathways, suggesting that ActRIIB 2.12-Fc has the ability to alter structural remodeling pathways and inflammation, which is consistent with anti-fibrotic activity.

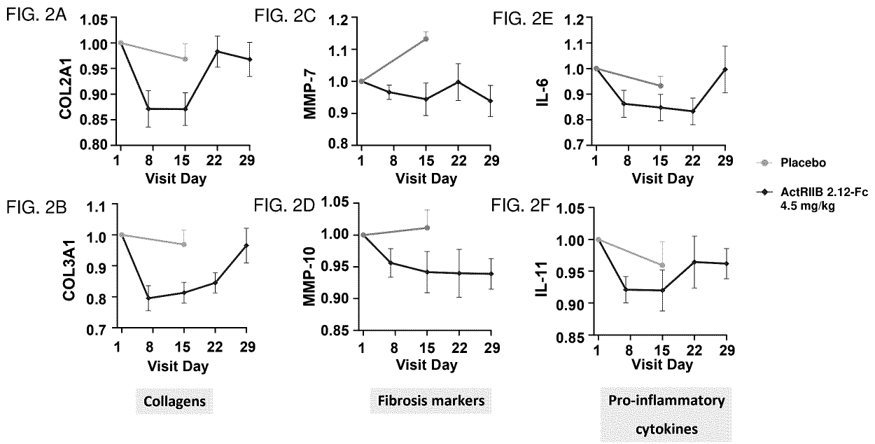

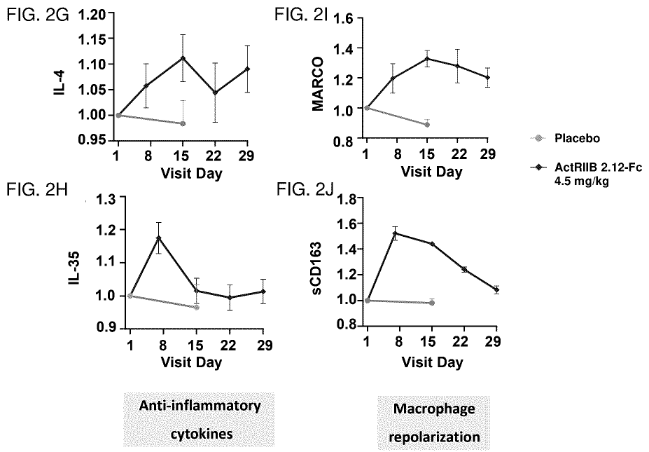

FIGS. 2A-2J show examples of proteins involved in extracellular matrix remodeling and inflammation. Fibrosis markers were decreased, as indicated by changes in matrix metalloproteinases (MMP-7 and MMP-10) and collagen fragments (FIGS. 2A-2D). In addition, a reduction in pro-inflammatory cytokines IL-6 and IL-11 was observed (FIGS. 2E-2F). An increase in anti-inflammatory cytokines (IL-4 and IL-35), and markers of macrophage polarization (MARCO and sCD163) were also observed (FIGS. 2G-2J).

### Example 2—Effect of Chimera 1/2b-mFc in a Mouse Model for Alport Syndrome

Four-week old B6129F1/J Col4a3^{-/-} mice underwent a 12-week treatment regimen with vehicle (n=6 (3 male, 3 female)), ramipril (n=4 (2 male, 2 female)), Chimera 1/2b-mFc (a homodimer of SEQ ID NO: 41 fused to a mouse Fc domain monomer by way of a GGG linker, n=7 (4 male, 3 female)), or Chimera 1/2b-mFc in combination with ramipril (n=4 (2 male, 2 female)). B6129F1/J mice are a first generation cross between C57BL/6 and 129S mouse strains. Vehicle (Tris buffered saline (25 mM Tris-HCl—pH 7.4, 150 mM sodium chloride—pH 7.4)) was administered twice weekly via intraperitoneal injection. Ramipril was administered at a dose of 20 mg/mL daily in drinking water. Chimera 1/2b-mFc was administered at a dose of 10 mg/kg twice weekly via intraperitoneal injection. The mice started treatment at 4 weeks of age and ended treatment at 16 weeks of age.

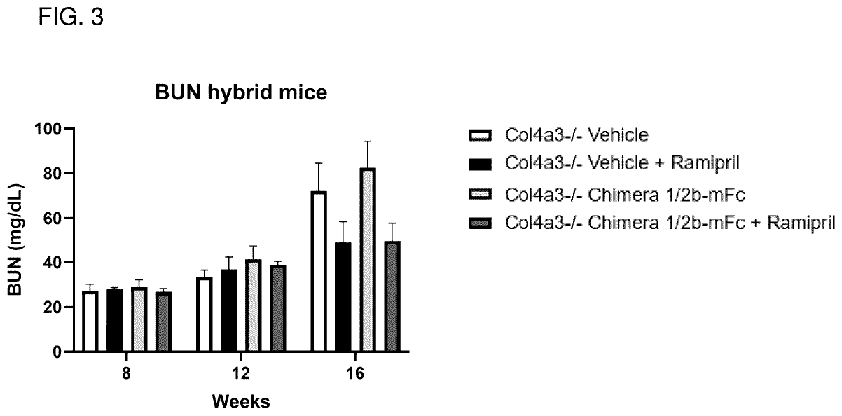

Blood urea nitrogen (BUN) levels were measured by QuantiChrom™ Urea Assay Kit (DIUR-100) per the manufacturer's protocol when the mice were aged 8, 12, and 16 weeks (i.e., after 4, 8, and 12 weeks of treatment, respectively). BUN results are shown in FIG. 3. Mouse age is shown on the x-axis. Data are shown as mean±SEM BUN in mg/dL.

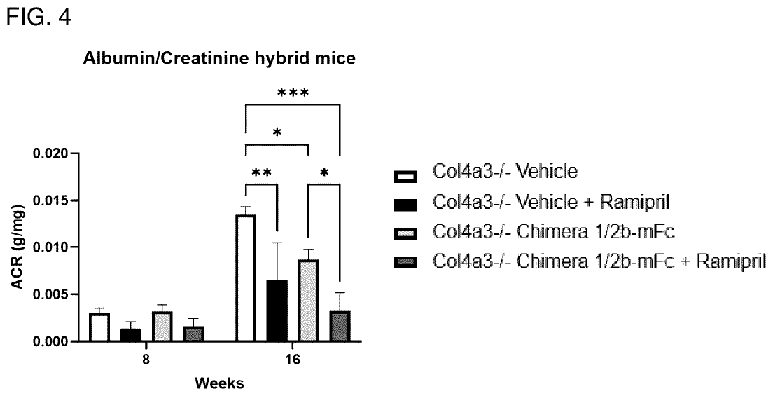

Urinary creatinine levels were measured when the mice were aged 8 and 16 weeks (i.e., after 4 and 12 weeks of treatment, respectively) using the QuantiChrom™ Creatinine Assay Kit (DICT-500). Urine albumin samples were analyzed when the mice were aged 8 and 16 weeks (i.e., after 4 and 12 weeks of treatment, respectively) by SDS-PAGE with the amounts of urine normalized by creatinine levels. Urinary albumin levels were quantified in Image J with Coomassie Blue-stained gel images (inverted) using 0.02-2 μg BSA loaded on the same gels as standards. Samples with high albumin concentrations were diluted and re-analyzed. Results are shown in FIG. 4, with data shown as mean±SEM urinary albumin/creatinine ratios (ACR) in g/mg. Mouse age is shown on the x-axis. Statistics are shown using 2-way ANOVA with a Tukey's multiple comparison test. *P<0.05, **P<0.01, ***P<0.001.

### Results

Ramipril but not Chimera 1/2b-mFc showed trends for reduction in BUN. Ramipril and Chimera 1/2b-mFc monotherapies significantly reduced urine albumin creatinine ratios (ACR) with further significant reduction in combination therapy when the mice were aged 16 weeks (i.e., after 12 weeks of treatment).

### Example 3—Evaluation of Activin Receptor IIA and IIB Ligand Traps in a Kidney Fibrosis Model

To evaluate efficacy of activin type II receptor A or B ligand traps, which inhibit both activin A and activin B, in attenuating kidney fibrosis, the unilateral ureteral obstruction (UUO) model of kidney fibrosis was used, which induces robust and reliable fibrosis. Male C57BL/6 mice were obtained from Charles River Laboratories. At 7-8 weeks of age, mice underwent UUO surgery. To create the model, the left ureter was ligated in close proximity to the renal pelvis with a 6′0 silk suture. Mice were then treated with IgG2a (n=6), ActRIIA-mFc (a homodimer of a wild-type human ActRIIA extracellular domain (SEQ ID NO: 44) fused to a mouse Fc domain monomer by way of a GGG linker, n=6), or ActRIIB-mFc (a homodimer of a wild-type human ActRIIB extracellular domain (SEQ ID NO: 45) fused to a mouse Fc domain monomer by way of a GGG linker, n=6).

Mice were dosed at 10 mg/kg at a dosing volume of 5 ml/kg in Tris-buffered saline (25 mM Tris, 150 mM NaCl, pH 7.4) intraperitoneally twice weekly. Treatment started on the day of model creation (day 0), with subsequent treatment given on day 3 and day 7. Mice were sacrificed on day 10. Buprenorphine SR (1 mg/kg daily) was given subcutaneously at the time of induction.

Mice were weighed daily throughout the study. At endpoint (day 10 post surgery), serum was collected, and the cortex of the UUO kidney was removed for assessment.

Formalin-fixed kidneys were embedded in paraffin, then sectioned at 4 μm. After deparaffinization, they were stained with Masson's trichrome to assess collagen content (Sigma Aldrich, catalogue no. HT15-1T), picrosirius red (PSR) to more specifically assess pathogenic collagens I and III (Polysciences, Inc., 24901-500), αSMA as a marker of activated, profibrotic fibroblasts (PIERCE, MA1-06110), fibronectin as a matrix protein characteristically increased in kidney fibrosis (Sigma, F3648) and phospho-Smad3 (pSmad3) S423/425 as a downstream mediator of activin signaling (Novus, NBP1-77836). Slides were counterstained with hematoxylin, dehydrated twice with 90% ethanol, cleared twice with xylene, coverslipped with mounting media, and then left to dry overnight before imaging.

Masson's trichrome, αSMA, fibronectin and pSmad3 were imaged under transmitted light and quantified using ImageJ. The Olympus® IX81 fluorescence microscope driven by MetaMorph® was used to image and quantify PSR staining. Images were quantified by measuring the percentage of positive area. All micrographs were captured at ×20 magnification.

Values are presented as mean±SEM. Statistical difference among multiple groups was determined using ANOVA with a Tukey's post hoc test. A P value<0.05 was considered significant, using GraphPad Prism 8 for calculations.

### Results

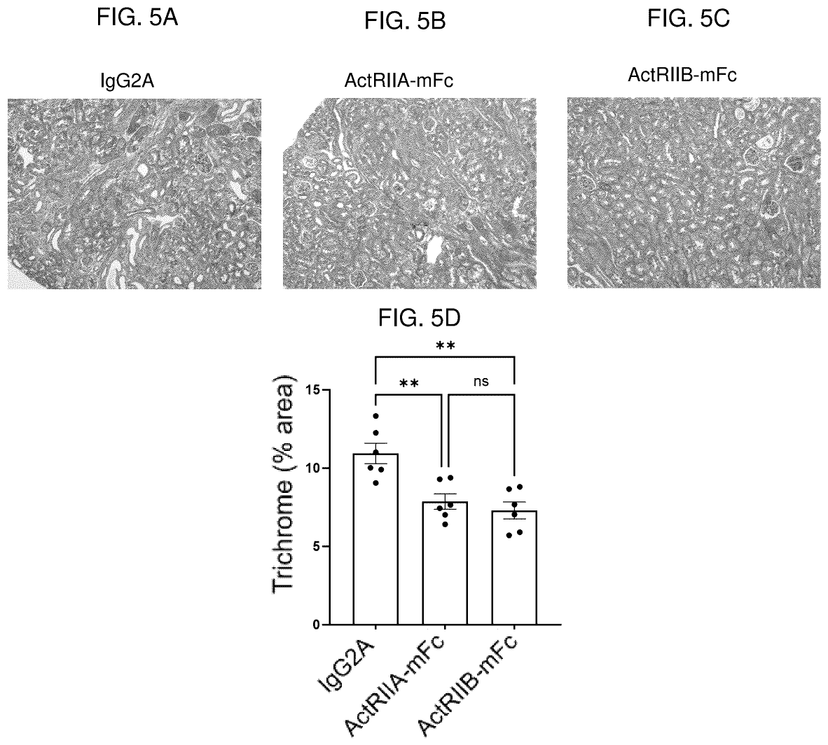

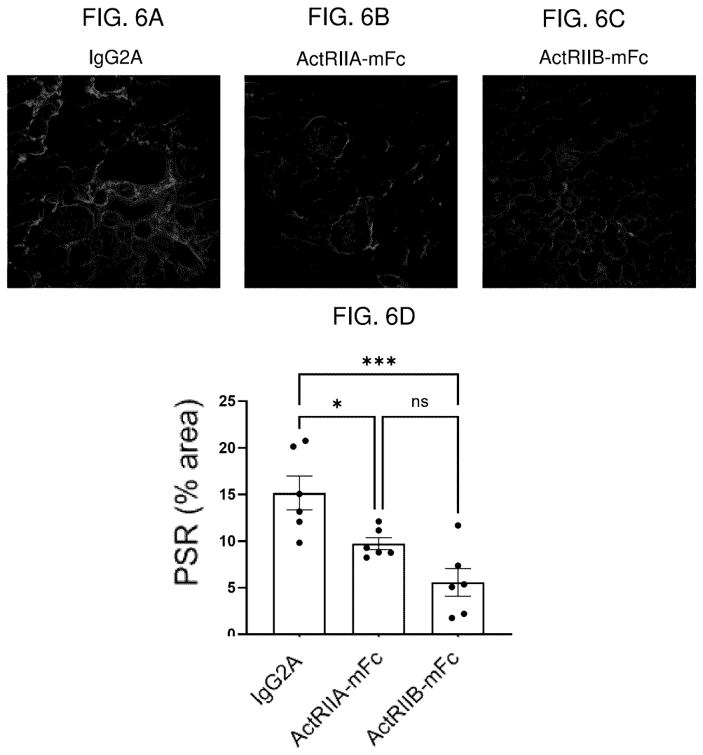

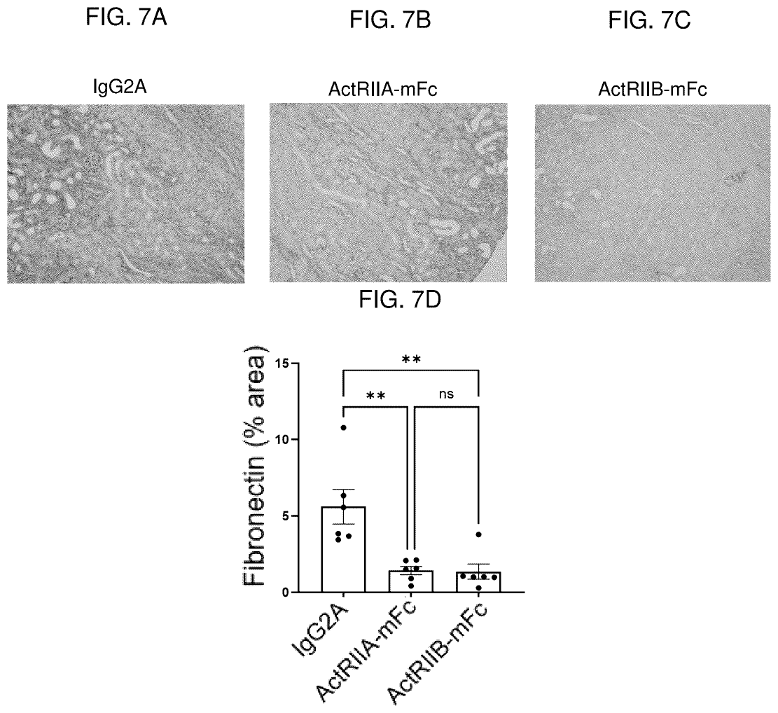

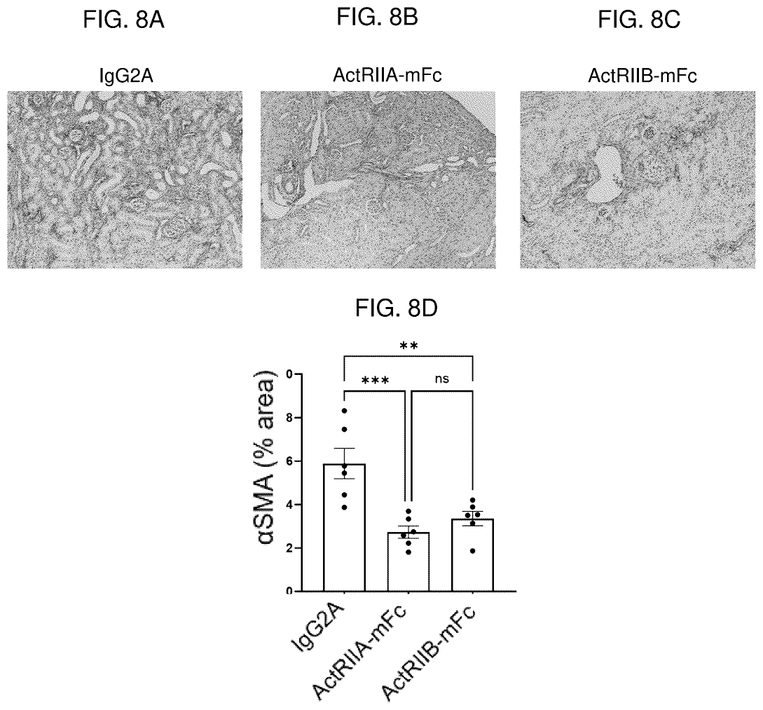

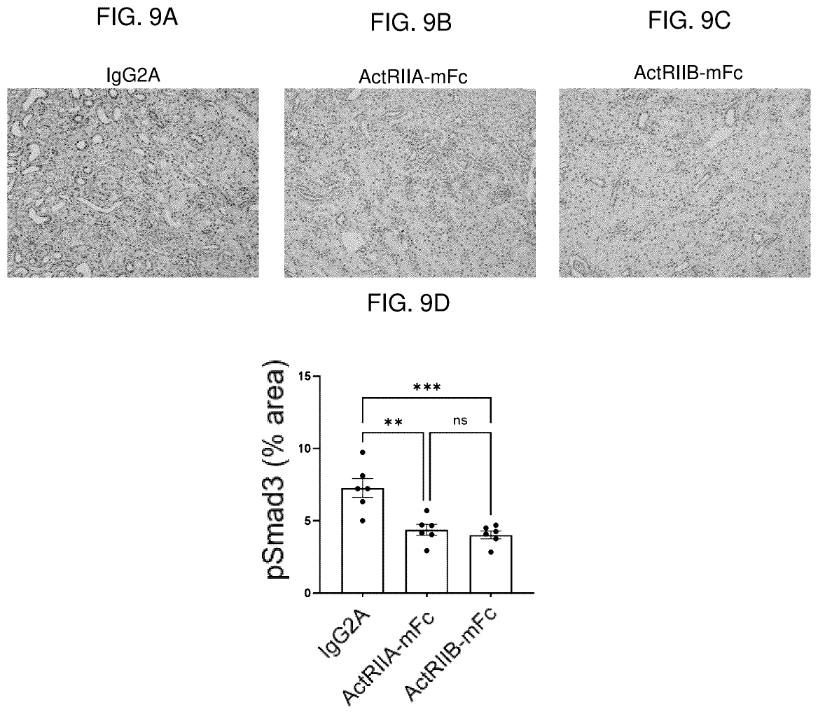

Trichrome staining results are shown in FIGS. 5A-5D. PSR staining results are shown in FIGS. 6A-6D. Fibronectin staining results are shown in FIGS. 7A-7D. αSMA staining results are shown in FIGS. 8A-8D. pSmad3 staining results are shown in FIGS. 9A-9D.

ActRIIA-mFc and ActRIIB-mFc attenuated UUO-induced kidney fibrosis. Fibrosis in UUO kidneys was assessed by Trichrome, PSR and IHC for the matrix protein fibronectin. As shown in FIGS. 5A-5D, FIGS. 6A-6D, and FIGS. 7A-7D, in all three assessments, fibrosis was significantly lower with administration of ActRIIA-mFc and ActRIIB-mFc. Fibroblast accumulation, as assessed by αSMA IHC, was also significantly lower with administration of ActRIIA-mFc and ActRIIB-mFc, as seen in FIGS. 8A-8D.

ActRIIA-mFc and ActRIIB-mFc also reduced Smad3 activation. Smad3 is a major downstream mediator of activin signaling. Phosphorylation at Ser423/425 is required for activity. This was assessed by IHC. As seen in FIGS. 9A-9D, both ActRIIA-mFc and ActRIIB-mFc significantly lowered Smad3 phosphorylation compared to IgG2a.

In summary, in the UUO model, both ActRIIA-mFc and ActRIIB-mFc attenuated markers of fibrosis, activation of fibroblasts, and Smad3 activation compared to IgG2a. These data support a protective role for type IIA or IIB receptor ligand traps in attenuating fibrosis, with no difference seen in efficacy between the two ligand traps.

### Example 4—Treatment of Alport Syndrome by Administration of an Extracellular ActRII Variant

According to the methods disclosed herein, a physician of skill in the art can treat a subject, such as a human patient, having Alport syndrome so as to reduce kidney fibrosis or slow the progression of kidney fibrosis, reduce kidney inflammation or slow the progression of kidney inflammation, or improve kidney function or slow the decline of kidney function. To treat the subject, a physician of skill in the art can administer to the subject a composition containing a polypeptide including an extracellular ActRII variant (e.g., a polypeptide including extracellular ActRII chimera having the sequence of any one of SEQ ID NOs: 1-43 (e.g., SEQ ID NOs: 22-43) or a polypeptide including an extracellular ActRIIB variant having the sequence of any one of SEQ ID NOs: 269-283 (e.g., SEQ ID NOs: 270-283), such as a polypeptide containing an extracellular ActRII chimera or an extracellular ActRIIB variant linked to an Fc domain monomer (e.g., a polypeptide that is in the form of a homodimer of an extracellular an extracellular ActRIIB variant or an extracellular ActRII chimera linked to an Fc domain monomer, which forms an Fc domain in the dimer)). The composition containing the extracellular ActRII variant may be administered to the subject, for example, by parenteral injection (e.g., intravenous or subcutaneous injection) to treat Alport syndrome. The polypeptide containing an extracellular ActRII variant is administered in a therapeutically effective amount, such as from 0.01 to 500 mg/kg (e.g., 0.01, 0.1, 0.2, 0.3, 0.325, 0.35, 0.375, 0.4, 0.5, 0.75, 1, 1.25, 1.5, 1.75, 2, 2.25, 2.5, 2.75, 3, 3.25, 3.5, 3.75, 4, 4.25, 4.5, 4.75, 5, 10, 15, 20, 25, 30, 35, 40, 45, 50, 100, 150, 200, 250, 300, 350, 400, 450, or 500 mg/kg). In some embodiments, the extracellular ActRII variant is administered once every sixteen weeks, quarterly, once every twelve weeks, bimonthly, once every eight weeks, once a month, once every four weeks, once every two weeks, or at least once a week or more (e.g., 1, 2, 3, 4, 5, 6, or 7 times a week or more). The extracellular ActRII variant is administered in an amount sufficient to increase reduce kidney fibrosis or slow the progression of kidney fibrosis, reduce kidney inflammation or slow the progression of kidney inflammation, improve GFR or slow a decline in GFR, and/or slow progression of the disease.

Following administration of the composition to a patient, a practitioner of skill in the art can monitor the patient's improvement in response to the therapy by a variety of methods. For example, a physician can monitor the patient's kidney function using blood tests, urine tests, and imaging tests. A finding that the patient's kidney function is improving or that the disease is progressing more slowly following administration of the composition compared to test results prior to administration of the composition indicates that the patient is responding favorably to the treatment. Subsequent doses can be determined and administered as needed.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents, and patent applications are herein incorporated by reference in their entirety to the same extent as if each individual publication, patent, or patent application was specifically and individually indicated to be incorporated by reference in its entirety.

Other embodiments are within the following claims.

1. A method of treating a subject having Alport syndrome, the method comprising administering to the subject a therapeutically effective amount of a composition of Table 7 or a composition of Table 8.

2. A method of reducing kidney fibrosis or slowing the progression of kidney fibrosis in a subject having Alport syndrome, the method comprising administering to the subject a therapeutically effective amount of a composition of Table 7 or a composition of Table 8.

3. A method of reducing kidney inflammation in a subject having Alport syndrome, the method comprising administering to the subject a therapeutically effective amount of a composition of Table 7 or a composition of Table 8.

4. A method of improving kidney function or slowing or inhibiting a decline in kidney function in a subject having Alport syndrome, the method comprising administering to the subject a therapeutically effective amount of a composition of Table 7 or a composition of Table 8.

5. The method of claim 4, wherein improving kidney function or slowing or inhibiting a decline in kidney function results in improved glomerular filtration rate (GFR) or slowing or stopping a progressive decline in GFR, improved protein-to-creatinine ratio (PCR), albumin-to-creatinine ratio (ACR), or urinary albumin-creatinine-ratio (UACR) or slowing or stopping a progressive increase in PCR, ACR or UACR, reduced blood urea nitrogen, reduced creatinine in the blood, improved creatinine clearance, or reduced proteinuria.

6. A method of delaying or inhibiting the onset of end-stage renal disease in a subject having Alport syndrome, comprising administering to the subject a therapeutically effective amount of a composition of Table 7 or a composition of Table 8.

7. The method of any one of claims 1-6, wherein the subject has X-linked Alport syndrome.

8. The method of any one of claims 1-6, wherein the subject has autosomal recessive Alport syndrome.

9. The method of any one of claims 1-6, wherein the subject has autosomal dominant Alport syndrome.

10. The method of any one of claims 1-9, wherein the subject has a mutation in COL4A3, a mutation in COL4A4, a mutation in COL4A5, or a mutation in both COL4A3 and COL4A4.

11. The method of any one of claims 1-10, wherein the subject is male.

12. The method of any one of claims 1-10, wherein the subject is female.

13. The method of any one of claims 1-12, wherein the method reduces kidney fibrosis or slows or inhibits progression of kidney fibrosis.

14. The method of any one of claims 1-13, wherein the method reduces kidney inflammation or slows or inhibits progression of kidney inflammation.

15. The method of any one of claims 1-14, wherein the method improves kidney function or slows or inhibits a decline in kidney function.

16. The method of any one of claims 1-15, wherein the method improves GFR or slows or inhibits a decline in GFR.

17. The method of any one of claims 1-16, wherein the method reduces hematuria, proteinuria, albuminuria, blood urea nitrogen, or creatinine in the blood.

18. The method of any one of claims 1-17, wherein the method delays or prevents the onset of end stage renal disease.

19. The method of any one of claims 1-18, wherein the method delays or prevents a need for dialysis, continuous renal replacement therapy, or a kidney transplant.

20. The method of any one of claims 1-19, wherein the method improves life expectancy for the subject.

21. The method of any one of claims 1-20, wherein the method comprises administering to the subject a therapeutically effective amount of a composition of Table 7.

22. The method of any one of claims 1-20, wherein the method comprises administering to the subject a therapeutically effective amount of a composition of Table 8.

23. The method of any one of claims 1-22, wherein the method further comprises administering to the subject an additional therapeutic agent.

24. The method of claim 23, wherein the additional therapeutic agent is an angiotensin II converting enzyme inhibitor, angiotensin II receptor blocker, beta-blocker, diuretic, angiotensin receptor-neprilysin inhibitor, calcium channel blocker, sodium-glucose cotransporter-2 inhibitor, ivabradine, HMG-CoA reductase inhibitor, aldosterone inhibitor, aliskiren, calcineurin inhibitor, endothelin receptor antagonist, sulodexide, vasopeptidase inhibitor, anti-transforming growth factor-β1 antibody, chemokine receptor 1 blocker, bone morphogenetic protein-7, PPARy agonist, or matrix metalloproteinase inhibitor.


SEQUENCE LISTING (284 sequences)

SEQ ID NO: 1 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 2 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 3 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 4 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 5 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 6 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 7 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 8 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 9 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 10 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 11 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 12 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 13 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 14 - protein, 107 aa, synthetic construct
  source 1..107: note = Synthetic Construct
  VARIANT 35: note = misc_feature - Xaa is Asp or Arg
  VARIANT 63: note = misc_feature - Xaa is Ile, Phe, Glu, Asp, Tyr, Ser, Asn, Gln, or Thr
  VARIANT 64: note = misc_feature - Xaa is Asn or Thr
  VARIANT 73: note = misc_feature - Xaa is Ala or Glu
  VARIANT 74: note = misc_feature - Xaa is Thr or Lys
  VARIANT 75: note = misc_feature - Xaa is Glu or Lys
  VARIANT 76: note = misc_feature - Xaa is Glu or Asp
  VARIANT 77: note = misc_feature - Xaa is Asn or Ser
  VARIANT 79: note = misc_feature - Xaa is Gln, Glu, Lys, Arg, Asp, or Asn
GRGEAETRECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 15 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRRHCFATWKNISGSIEIVKQGCWLDDXXCYDRTDCVXXXXXPXVYFCCCEGNMCNEKFSYFPEMEVTQPTS

SEQ ID NO: 16 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCFATWKNISGSIEIVKQGCWLDDXXCYDRTDCVXXXXXPXVYFCCCEGNMCNEKFSYFPEMEVTQPTS

SEQ ID NO: 17 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCYASWKNISGSIEIVKQGCWLDDXXCYDRTDCVXXXXXPXVYFCCCEGNMCNEKFSYFPEMEVTQPTS

SEQ ID NO: 18 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCYASWRNSSGSIEIVKQGCWLDDXXCYDRTDCVXXXXXPXVYFCCCEGNMCNEKFSYFPEMEVTQPTS

SEQ ID NO: 19 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCYASWRNSSGTIEIVKQGCWLDDXXCYDRTDCVXXXXXPXVYFCCCEGNMCNEKFSYFPEMEVTQPTS

SEQ ID NO: 20 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRTDCVXXXXXPXVYFCCCEGNMCNEKFSYFPEMEVTQPTS

SEQ ID NO: 21 - protein, 109 aa, synthetic construct
  source 1..109: note = Synthetic Construct
  VARIANT 37: note = misc_feature - Xaa is Asp or Arg
  VARIANT 65: note = misc_feature - Xaa is Ile, Phe, Glu, Asp, Tyr, Ser, Asn, Gln, or Thr
  VARIANT 66: note = misc_feature - Xaa is Asn or Thr
  VARIANT 75: note = misc_feature - Xaa is Ala or Glu
  VARIANT 76: note = misc_feature - Xaa is Thr or Lys
  VARIANT 77: note = misc_feature - Xaa is Glu or Lys
  VARIANT 78: note = misc_feature - Xaa is Glu or Asp
  VARIANT 79: note = misc_feature - Xaa is Asn or Ser
  VARIANT 81: note = misc_feature - Xaa is Gln, Glu, Lys, Arg, Asp, or Asn
GAILGRSETQECIYYNANWELERTNQSGLERCEGEQXKRLHCYASWRNSSGTIELVKKGCWLDDXXCYDRQECVXXXXXPXVYFCCCEGNMCNEKFSYFPEMEVTQPTS

SEQ ID NO: 22 - protein, 109 aa, synthetic construct
  source 1..109: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 23 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 24 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 25 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 26 - protein, 109 aa, synthetic construct
  source 1..109: note = Synthetic Construct
GAILGRAETRECIYYNANWELERTNQSGLERCEGEQDKRLHCYASWRNSSGTIEIVKQGCWLDDFNCYDRTDCVATEENPQVYFCCCEGNMCNEKFSYFPEMEVTQPTS

SEQ ID NO: 27 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDFNCYDRTDCVEKKDSPEVYFCCCEGNMCNEKFSYFPEMEVTQPTS

SEQ ID NO: 28 - protein, 107 aa, synthetic construct
  source 1..107: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 29 - protein, 107 aa, synthetic construct
  source 1..107: note = Synthetic Construct
GRGEAETRECIYYNANWELERTNQSGLERCEGEQDKRLHCFATWKNISGSIEIVKQGCWLDDFNCYDRTDCVEKKDSPQVYFCCCEGNMCNEKFSYFPEMEVTQPTS

SEQ ID NO: 30 - protein, 107 aa, synthetic construct
  source 1..107: note = Synthetic Construct
GRGEAETRECIYYNANWELERTNQSGLERCEGEQDKRLHCYASWKNISGSIEIVKQGCWLDDFNCYDRTDCVEKKDSPQVYFCCCEGNMCNEKFSYFPEMEVTQPTS

SEQ ID NO: 31 - protein, 107 aa, synthetic construct
  source 1..107: note = Synthetic Construct
GRGEAETRECIYYNANWELERTNQSGLERCEGEQDKRLHCYASWRNSSGSIEIVKQGCWLDDFNCYDRTDCVEKKDSPQVYFCCCEGNMCNEKFSYFPEMEVTQPTS

SEQ ID NO: 32 - protein, 107 aa, synthetic construct
  source 1..107: note = Synthetic Construct
GRGEAETRECIYYNANWELERTNQSGLERCEGEQDKRLHCYASWRNSSGTIELVKKGCWLDDFNCYDRTDCVEKKDSPQVYFCCCEGNMCNEKFSYFPEMEVTQPTS

SEQ ID NO: 33 - protein, 107 aa, synthetic construct
  source 1..107: note = Synthetic Construct
GRGEAETRECIYYNANWELERTNQSGLERCEGEQDKRLHCYASWRNSSGTIELVKKGCWLDDFNCYDRQECVATKDSPQVYFCCCEGNMCNEKFSYFPEMEVTQPTS

SEQ ID NO: 34 - protein, 107 aa, synthetic construct
  source 1..107: note = Synthetic Construct
GRGEAETRECIYYNANWELERTNQSGLERCEGEQDKRLHCYASWRNSSGTIELVKKGCWLDDFNCYDRQECVATEENPQVYFCCCEGNMCNEKFSYFPEMEVTQPTS

SEQ ID NO: 35 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDNNCYDRTDCVEKKDSPQVYFCCCEGNMCNEKFSYFPEMEVTQPTS

SEQ ID NO: 36 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDTNCYDRTDCVEKKDSPQVYFCCCEGNMCNEKFSYFPEMEVTQPTS

SEQ ID NO: 37 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 38 - protein, 107 aa, synthetic construct
  source 1..107: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTS

SEQ ID NO: 39 - protein, 107 aa, synthetic construct
  source 1..107: note = Synthetic Construct
GRGEAETRECIYYNANWELERTNQSGLERCEGEQDKRLHCYASWRNSSGTIEIVKQGCWLDDFNCYDRTDCVETKDSPQVYFCCCEGNMCNEKFSYFPEMEVTQPTS

SEQ ID NO: 40 - protein, 109 aa, synthetic construct
  source 1..109: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 41 - protein, 109 aa, synthetic construct
  source 1..109: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 42 - protein, 107 aa, synthetic construct
  source 1..107: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 43 - protein, 107 aa, synthetic construct
  source 1..107: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 44 - protein, 109 aa, Homo sapiens
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 45 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 46 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 47 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 48 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 49 - protein, 4 aa, synthetic construct
  source 1..4: note = Synthetic Construct
GGGA

SEQ ID NO: 50 - protein, 4 aa, synthetic construct
  source 1..4: note = Synthetic Construct
GGGS

SEQ ID NO: 51 - protein, 4 aa, synthetic construct
  source 1..4: note = Synthetic Construct
GGGG

SEQ ID NO: 52 - protein, 5 aa, synthetic construct
  source 1..5: note = Synthetic Construct
GGGGA

SEQ ID NO: 53 - protein, 5 aa, synthetic construct
  source 1..5: note = Synthetic Construct
GGGGS

SEQ ID NO: 54 - protein, 5 aa, synthetic construct
  source 1..5: note = Synthetic Construct
GGGGG

SEQ ID NO: 55 - protein, 4 aa, synthetic construct
  source 1..4: note = Synthetic Construct
GGAG

SEQ ID NO: 56 - protein, 4 aa, synthetic construct
  source 1..4: note = Synthetic Construct
GGSG

SEQ ID NO: 57 - protein, 4 aa, synthetic construct
  source 1..4: note = Synthetic Construct
AGGG

SEQ ID NO: 58 - protein, 4 aa, synthetic construct
  source 1..4: note = Synthetic Construct
SGGG

SEQ ID NO: 59 - protein, 4 aa, synthetic construct
  source 1..4: note = Synthetic Construct
GAGA

SEQ ID NO: 60 - protein, 4 aa, synthetic construct
  source 1..4: note = Synthetic Construct
GSGS

SEQ ID NO: 61 - protein, 6 aa, synthetic construct
  source 1..6: note = Synthetic Construct
GAGAGA

SEQ ID NO: 62 - protein, 6 aa, synthetic construct
  source 1..6: note = Synthetic Construct
GSGSGS

SEQ ID NO: 63 - protein, 8 aa, synthetic construct
  source 1..8: note = Synthetic Construct
GAGAGAGA

SEQ ID NO: 64 - protein, 8 aa, synthetic construct
  source 1..8: note = Synthetic Construct
GSGSGSGS

SEQ ID NO: 65 - protein, 10 aa, synthetic construct
  source 1..10: note = Synthetic Construct
GAGAGAGAGA

SEQ ID NO: 66 - protein, 10 aa, synthetic construct
  source 1..10: note = Synthetic Construct
GSGSGSGSGS

SEQ ID NO: 67 - protein, 12 aa, synthetic construct
  source 1..12: note = Synthetic Construct
GAGAGAGAGAGA

SEQ ID NO: 68 - protein, 12 aa, synthetic construct
  source 1..12: note = Synthetic Construct
GSGSGSGSGSGS

SEQ ID NO: 69 - protein, 6 aa, synthetic construct
  source 1..6: note = Synthetic Construct
GGAGGA

SEQ ID NO: 70 - protein, 6 aa, synthetic construct
  source 1..6: note = Synthetic Construct
GGSGGS

SEQ ID NO: 71 - protein, 9 aa, synthetic construct
  source 1..9: note = Synthetic Construct
GGAGGAGGA

SEQ ID NO: 72 - protein, 9 aa, synthetic construct
  source 1..9: note = Synthetic Construct
GGSGGSGGS

SEQ ID NO: 73 - protein, 12 aa, synthetic construct
  source 1..12: note = Synthetic Construct
GGAGGAGGAGGA

SEQ ID NO: 74 - protein, 12 aa, synthetic construct
  source 1..12: note = Synthetic Construct
GGSGGSGGSGGS

SEQ ID NO: 75 - protein, 8 aa, synthetic construct
  source 1..8: note = Synthetic Construct
GGAGGGAG

SEQ ID NO: 76 - protein, 8 aa, synthetic construct
  source 1..8: note = Synthetic Construct
GGSGGGSG

SEQ ID NO: 77 - protein, 12 aa, synthetic construct
  source 1..12: note = Synthetic Construct
GGAGGGAGGGAG

SEQ ID NO: 78 - protein, 12 aa, synthetic construct
  source 1..12: note = Synthetic Construct
GGSGGGSGGGSG

SEQ ID NO: 79 - protein, 15 aa, synthetic construct
  source 1..15: note = Synthetic Construct
GGGGAGGGGAGGGGA

SEQ ID NO: 80 - protein, 15 aa, synthetic construct
  source 1..15: note = Synthetic Construct
GGGGSGGGGSGGGGS

SEQ ID NO: 81 - protein, 5 aa, synthetic construct
  source 1..5: note = Synthetic Construct
GGGAG

SEQ ID NO: 82 - protein, 6 aa, synthetic construct
  source 1..6: note = Synthetic Construct
GGGAGG

SEQ ID NO: 83 - protein, 7 aa, synthetic construct
  source 1..7: note = Synthetic Construct
GGGAGGG

SEQ ID NO: 84 - protein, 4 aa, synthetic construct
  source 1..4: note = Synthetic Construct
AAAL

SEQ ID NO: 85 - protein, 4 aa, synthetic construct
  source 1..4: note = Synthetic Construct
AAAK

SEQ ID NO: 86 - protein, 4 aa, synthetic construct
  source 1..4: note = Synthetic Construct
AAAR

SEQ ID NO: 87 - protein, 14 aa, synthetic construct
  source 1..14: note = Synthetic Construct
EGKSSGSGSESKST

SEQ ID NO: 88 - protein, 12 aa, synthetic construct
  source 1..12: note = Synthetic Construct
GSAGSAAGSGEF

SEQ ID NO: 89 - protein, 12 aa, synthetic construct
  source 1..12: note = Synthetic Construct
AEAAAKEAAAKA

SEQ ID NO: 90 - protein, 18 aa, synthetic construct
  source 1..18: note = Synthetic Construct
KESGSVSSEQLAQFRSLD

SEQ ID NO: 91 - protein, 10 aa, synthetic construct
  source 1..10: note = Synthetic Construct
GENLYFQSGG

SEQ ID NO: 92 - protein, 8 aa, synthetic construct
  source 1..8: note = Synthetic Construct
SACYCELS

SEQ ID NO: 93 - protein, 5 aa, synthetic construct
  source 1..5: note = Synthetic Construct
RSIAT

SEQ ID NO: 94 - protein, 17 aa, synthetic construct
  source 1..17: note = Synthetic Construct
RPACKIPNDLKQKVMNH

SEQ ID NO: 95 - protein, 36 aa, synthetic construct
  source 1..36: note = Synthetic Construct
GGSAGGSGSGSSGGSSGASGTGTAGGTGSGSGTGSG

SEQ ID NO: 96 - protein, 17 aa, synthetic construct
  source 1..17: note = Synthetic Construct
AAANSSIDLISVPVDSR

SEQ ID NO: 97 - protein, 36 aa, synthetic construct
  source 1..36: note = Synthetic Construct
GGSGGGSEGGGSEGGGSEGGGSEGGGSEGGGSGGGS

SEQ ID NO: 98 - protein, 5 aa, synthetic construct
  source 1..5: note = Synthetic Construct
EAAAK

SEQ ID NO: 99 - protein, 5 aa, synthetic construct
  source 1..5: note = Synthetic Construct
PAPAP

SEQ ID NO: 100 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101-103 - skipped (3 entries)

SEQ ID NO: 104 - protein, 6 aa, synthetic construct
  source 1..6: note = Synthetic Construct
NPVTPK

SEQ ID NO: 105-106 - skipped (2 entries)

SEQ ID NO: 107 - protein, 339 aa, synthetic construct
  source 1..339: note = Synthetic Construct
GAILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 337 aa, synthetic construct
  source 1..337: note = Synthetic Construct
GRGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 337 aa, synthetic construct
  source 1..337: note = Synthetic Construct
GRGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 337 aa, synthetic construct
  source 1..337: note = Synthetic Construct
GRGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 107 aa, synthetic construct
  source 1..107: note = Synthetic Construct
ILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 112 - protein, 106 aa, synthetic construct
  source 1..106: note = Synthetic Construct
RGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 113 - protein, 106 aa, synthetic construct
  source 1..106: note = Synthetic Construct
RGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 114 - protein, 106 aa, synthetic construct
  source 1..106: note = Synthetic Construct
RGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 115 - protein, 106 aa, synthetic construct
  source 1..106: note = Synthetic Construct
LGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 116 - protein, 105 aa, synthetic construct
  source 1..105: note = Synthetic Construct
GEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 117 - protein, 105 aa, synthetic construct
  source 1..105: note = Synthetic Construct
GEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 118 - protein, 105 aa, synthetic construct
  source 1..105: note = Synthetic Construct
GEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 119 - protein, 105 aa, synthetic construct
  source 1..105: note = Synthetic Construct
GRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 120 - protein, 104 aa, synthetic construct
  source 1..104: note = Synthetic Construct
EAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 121 - protein, 104 aa, synthetic construct
  source 1..104: note = Synthetic Construct
EAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 122 - protein, 104 aa, synthetic construct
  source 1..104: note = Synthetic Construct
EAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 123 - protein, 104 aa, synthetic construct
  source 1..104: note = Synthetic Construct
RAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 124 - protein, 103 aa, synthetic construct
  source 1..103: note = Synthetic Construct
AETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 125 - protein, 103 aa, synthetic construct
  source 1..103: note = Synthetic Construct
AETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 126 - protein, 103 aa, synthetic construct
  source 1..103: note = Synthetic Construct
AETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 127 - protein, 103 aa, synthetic construct
  source 1..103: note = Synthetic Construct
AETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 128 - protein, 102 aa, synthetic construct
  source 1..102: note = Synthetic Construct
ETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 129 - protein, 102 aa, synthetic construct
  source 1..102: note = Synthetic Construct
ETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 130 - protein, 102 aa, synthetic construct
  source 1..102: note = Synthetic Construct
ETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 131 - protein, 102 aa, synthetic construct
  source 1..102: note = Synthetic Construct
ETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 132 - protein, 101 aa, synthetic construct
  source 1..101: note = Synthetic Construct
TRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 133 - protein, 101 aa, synthetic construct
  source 1..101: note = Synthetic Construct
TRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 134 - protein, 101 aa, synthetic construct
  source 1..101: note = Synthetic Construct
TRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 135 - protein, 101 aa, synthetic construct
  source 1..101: note = Synthetic Construct
TRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 136 - protein, 100 aa, synthetic construct
  source 1..100: note = Synthetic Construct
RECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTS

SEQ ID NO: 137 - protein, 100 aa, synthetic construct
  source 1..100: note = Synthetic Construct
RECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTS

SEQ ID NO: 138 - protein, 100 aa, synthetic construct
  source 1..100: note = Synthetic Construct
RECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 139 - protein, 100 aa, synthetic construct
  source 1..100: note = Synthetic Construct
RECIYYNANWELERTNQSGLERCEGEQDKRLHCYASWRNSSGTIEIVKQGCWLDDINCYDRTDCVATEENPQVYFCCCEGNMCNEKFSYFPEMEVTQPTS

SEQ ID NO: 140 - protein, 106 aa, synthetic construct
  source 1..106: note = Synthetic Construct
RGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 141 - protein, 106 aa, synthetic construct
  source 1..106: note = Synthetic Construct
RGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 142 - protein, 107 aa, synthetic construct
  source 1..107: note = Synthetic Construct
ILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 143 - protein, 107 aa, synthetic construct
  source 1..107: note = Synthetic Construct
ILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 144 - protein, 106 aa, synthetic construct
  source 1..106: note = Synthetic Construct
RGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 145 - protein, 106 aa, synthetic construct
  source 1..106: note = Synthetic Construct
RGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 146 - protein, 105 aa, synthetic construct
  source 1..105: note = Synthetic Construct
GEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 147 - protein, 105 aa, synthetic construct
  source 1..105: note = Synthetic Construct
GEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 148 - protein, 106 aa, synthetic construct
  source 1..106: note = Synthetic Construct
LGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 149 - protein, 106 aa, synthetic construct
  source 1..106: note = Synthetic Construct
LGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 150 - protein, 105 aa, synthetic construct
  source 1..105: note = Synthetic Construct
GEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 151 - protein, 105 aa, synthetic construct
  source 1..105: note = Synthetic Construct
GEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 152 - protein, 104 aa, synthetic construct
  source 1..104: note = Synthetic Construct
EAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 153 - protein, 104 aa, synthetic construct
  source 1..104: note = Synthetic Construct
EAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 154 - protein, 105 aa, synthetic construct
  source 1..105: note = Synthetic Construct
GRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 155 - protein, 105 aa, synthetic construct
  source 1..105: note = Synthetic Construct
GRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 156 - protein, 104 aa, synthetic construct
  source 1..104: note = Synthetic Construct
EAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 157 - protein, 104 aa, synthetic construct
  source 1..104: note = Synthetic Construct
EAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 158 - protein, 103 aa, synthetic construct
  source 1..103: note = Synthetic Construct
AETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 159 - protein, 103 aa, synthetic construct
  source 1..103: note = Synthetic Construct
AETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 160 - protein, 104 aa, synthetic construct
  source 1..104: note = Synthetic Construct
RAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 161 - protein, 104 aa, synthetic construct
  source 1..104: note = Synthetic Construct
RSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 162 - protein, 103 aa, synthetic construct
  source 1..103: note = Synthetic Construct
AETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 163 - protein, 103 aa, synthetic construct
  source 1..103: note = Synthetic Construct
AETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 164 - protein, 102 aa, synthetic construct
  source 1..102: note = Synthetic Construct
ETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 165 - protein, 102 aa, synthetic construct
  source 1..102: note = Synthetic Construct
ETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 166 - protein, 103 aa, synthetic construct; identical to SEQ ID NO: 126
  source 1..103: note = Synthetic Construct

SEQ ID NO: 167 - protein, 103 aa, synthetic construct
  source 1..103: note = Synthetic Construct
SETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 168 - protein, 102 aa, synthetic construct
  source 1..102: note = Synthetic Construct
ETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 169 - protein, 102 aa, synthetic construct
  source 1..102: note = Synthetic Construct
ETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 170 - protein, 101 aa, synthetic construct
  source 1..101: note = Synthetic Construct
TRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 171 - protein, 101 aa, synthetic construct
  source 1..101: note = Synthetic Construct
TRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 172 - protein, 102 aa, synthetic construct; identical to SEQ ID NO: 130
  source 1..102: note = Synthetic Construct

SEQ ID NO: 173 - protein, 102 aa, synthetic construct
  source 1..102: note = Synthetic Construct
ETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 174 - protein, 101 aa, synthetic construct
  source 1..101: note = Synthetic Construct
TRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 175 - protein, 101 aa, synthetic construct
  source 1..101: note = Synthetic Construct
TRECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 176 - protein, 100 aa, synthetic construct
  source 1..100: note = Synthetic Construct
RECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTS

SEQ ID NO: 177 - protein, 100 aa, synthetic construct
  source 1..100: note = Synthetic Construct
RECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTS

SEQ ID NO: 178 - protein, 101 aa, synthetic construct; identical to SEQ ID NO: 134
  source 1..101: note = Synthetic Construct

SEQ ID NO: 179 - protein, 101 aa, synthetic construct
  source 1..101: note = Synthetic Construct
TQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 180 - protein, 100 aa, synthetic construct
  source 1..100: note = Synthetic Construct
RECIYYNANWELERTNQSGLERCEGEQDKRLHCYASWRNSSGTIELVKKGCWLDDFNCYDRQECVATKDSPEVYFCCCEGNMCNEKFSYFPEMEVTQPTS

SEQ ID NO: 181 - protein, 100 aa, synthetic construct
  source 1..100: note = Synthetic Construct
RECIYYNANWELERTNQSGLERCEGEQDKRLHCYASWRNSSGTIELVKKGCWLDDFNCYDRQECVATKENPQVYFCCCEGNMCNEKFSYFPEMEVTQPTS

SEQ ID NO: 182 - protein, 100 aa, synthetic construct; identical to SEQ ID NO: 138
  source 1..100: note = Synthetic Construct

SEQ ID NO: 183 - protein, 100 aa, synthetic construct
  source 1..100: note = Synthetic Construct
QECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTS

SEQ ID NO: 184 - protein, 337 aa, synthetic construct
  source 1..337: note = Synthetic Construct
ILGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 185 - protein, 336 aa, synthetic construct
  source 1..336: note = Synthetic Construct
RGEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 186 - protein, 336 aa, synthetic construct
  source 1..336: note = Synthetic Construct
RGEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 336 aa, synthetic construct
  source 1..336: note = Synthetic Construct
RGEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 188 - protein, 336 aa, synthetic construct
  source 1..336: note = Synthetic Construct
LGRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 335 aa, synthetic construct
  source 1..335: note = Synthetic Construct
GEAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 335 aa, synthetic construct
  source 1..335: note = Synthetic Construct
GEAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 335 aa, synthetic construct
  source 1..335: note = Synthetic Construct
GEAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - protein, 335 aa, synthetic construct
  source 1..335: note = Synthetic Construct
GRAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 334 aa, synthetic construct
  source 1..334: note = Synthetic Construct
EAETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 334 aa, synthetic construct
  source 1..334: note = Synthetic Construct
EAETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 334 aa, synthetic construct
  source 1..334: note = Synthetic Construct
EAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 334 aa, synthetic construct
  source 1..334: note = Synthetic Construct
RAETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 333 aa, synthetic construct
  source 1..333: note = Synthetic Construct
AETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 333 aa, synthetic construct
  source 1..333: note = Synthetic Construct
AETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 333 aa, synthetic construct
  source 1..333: note = Synthetic Construct
AETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 333 aa, synthetic construct
  source 1..333: note = Synthetic Construct
AETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 332 aa, synthetic construct
  source 1..332: note = Synthetic Construct
ETRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 332 aa, synthetic construct
  source 1..332: note = Synthetic Construct
ETRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 332 aa, synthetic construct
  source 1..332: note = Synthetic Construct
ETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 332 aa, synthetic construct
  source 1..332: note = Synthetic Construct
ETRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 331 aa, synthetic construct
  source 1..331: note = Synthetic Construct
TRECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 331 aa, synthetic construct
  source 1..331: note = Synthetic Construct
TRECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 331 aa, synthetic construct
  source 1..331: note = Synthetic Construct
TRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 331 aa, synthetic construct
  source 1..331: note = Synthetic Construct
TRECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 330 aa, synthetic construct
  source 1..330: note = Synthetic Construct
RECIYYNANWELERTNQSGLERCEGEQDKRLHCYASWRNSSGTIEIVKQGCWLDDINCYDRTDCVEKKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 330 aa, synthetic construct
  source 1..330: note = Synthetic Construct
RECIYYNANWELERTNQSGLERCEGEQDKRLHCYASWRNSSGTIEIVKQGCWLDDINCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 330 aa, synthetic construct
  source 1..330: note = Synthetic Construct
RECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 330 aa, synthetic construct
  source 1..330: note = Synthetic Construct
RECIYYNANWELERTNQSGLERCEGEQDKRLHCYASWRNSSGTIEIVKQGCWLDDINCYDRTDCVATEENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 337 aa, synthetic construct
  source 1..337: note = Synthetic Construct
GRGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 337 aa, synthetic construct
  source 1..337: note = Synthetic Construct
GRGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 339 aa, synthetic construct
  source 1..339: note = Synthetic Construct
GAILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 339 aa, synthetic construct
  source 1..339: note = Synthetic Construct
GAILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 337 aa, synthetic construct
  source 1..337: note = Synthetic Construct
GRGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 337 aa, synthetic construct
  source 1..337: note = Synthetic Construct
GRGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 219 - protein, 336 aa, synthetic construct
  source 1..336: note = Synthetic Construct
RGEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 220 - protein, 336 aa, synthetic construct
  source 1..336: note = Synthetic Construct
RGEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 221 - protein, 337 aa, synthetic construct
  source 1..337: note = Synthetic Construct
ILGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 222 - protein, 337 aa, synthetic construct
  source 1..337: note = Synthetic Construct
ILGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 223 - protein, 336 aa, synthetic construct
  source 1..336: note = Synthetic Construct
RGEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 336 aa, synthetic construct
  source 1..336: note = Synthetic Construct
RGEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 225 - protein, 335 aa, synthetic construct
  source 1..335: note = Synthetic Construct
GEAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 226 - protein, 335 aa, synthetic construct
  source 1..335: note = Synthetic Construct
GEAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 227 - protein, 336 aa, synthetic construct
  source 1..336: note = Synthetic Construct
LGRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 228 - protein, 336 aa, synthetic construct
  source 1..336: note = Synthetic Construct
LGRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 335 aa, synthetic construct
  source 1..335: note = Synthetic Construct
GEAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 230 - protein, 335 aa, synthetic construct
  source 1..335: note = Synthetic Construct
GEAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 334 aa, synthetic construct
  source 1..334: note = Synthetic Construct
EAETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 232 - protein, 334 aa, synthetic construct
  source 1..334: note = Synthetic Construct
EAETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 233 - protein, 335 aa, synthetic construct
  source 1..335: note = Synthetic Construct
GRAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 234 - protein, 335 aa, synthetic construct
  source 1..335: note = Synthetic Construct
GRSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 235 - protein, 334 aa, synthetic construct
  source 1..334: note = Synthetic Construct
EAETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 334 aa, synthetic construct
  source 1..334: note = Synthetic Construct
EAETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 237 - protein, 333 aa, synthetic construct
  source 1..333: note = Synthetic Construct
AETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 238 - protein, 333 aa, synthetic construct
  source 1..333: note = Synthetic Construct
AETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 239 - protein, 334 aa, synthetic construct
  source 1..334: note = Synthetic Construct
RAETRECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 334 aa, synthetic construct
  source 1..334: note = Synthetic Construct
RSETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 241 - protein, 333 aa, synthetic construct
  source 1..333: note = Synthetic Construct
AETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 242 - protein, 333 aa, synthetic construct
  source 1..333: note = Synthetic Construct
AETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 332 aa, synthetic construct
  source 1..332: note = Synthetic Construct
ETRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 244 - protein, 332 aa, synthetic construct
  source 1..332: note = Synthetic Construct
ETRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 245 - protein, 333 aa, synthetic construct; identical to SEQ ID NO: 199
  source 1..333: note = Synthetic Construct

SEQ ID NO: 246 - protein, 333 aa, synthetic construct
  source 1..333: note = Synthetic Construct
SETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 247 - protein, 332 aa, synthetic construct
  source 1..332: note = Synthetic Construct
ETRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 248 - protein, 332 aa, synthetic construct
  source 1..332: note = Synthetic Construct
ETRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 331 aa, synthetic construct
  source 1..331: note = Synthetic Construct
TRECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 331 aa, synthetic construct
  source 1..331: note = Synthetic Construct
TRECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 330 aa, synthetic construct
  source 1..330: note = Synthetic Construct
QECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 252 - protein, 332 aa, synthetic construct; identical to SEQ ID NO: 203
  source 1..332: note = Synthetic Construct

SEQ ID NO: 253 - protein, 332 aa, synthetic construct
  source 1..332: note = Synthetic Construct
ETQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 254 - protein, 331 aa, synthetic construct
  source 1..331: note = Synthetic Construct
TRECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 255 - protein, 331 aa, synthetic construct
  source 1..331: note = Synthetic Construct
TRECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 256 - protein, 330 aa, synthetic construct
  source 1..330: note = Synthetic Construct
RECIYYNANWELERTNQSGLERCEGEQDKRRHCFATWKNISGSIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 257 - protein, 330 aa, synthetic construct
  source 1..330: note = Synthetic Construct
RECIYYNANWELERTNQSGLERCEGEQDKRLHCYASWRNSSGTIEIVKQGCWLDDETCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 258 - protein, 331 aa, synthetic construct; identical to SEQ ID NO: 207
  source 1..331: note = Synthetic Construct

SEQ ID NO: 259 - protein, 331 aa, synthetic construct
  source 1..331: note = Synthetic Construct
TQECIYYNANWELERTNQSGLERCEGEQDKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 330 aa, synthetic construct
  source 1..330: note = Synthetic Construct
RECIYYNANWELERTNQSGLERCEGEQDKRLHCYASWRNSSGTIELVKKGCWLDDFNCYDRQECVATKDSPEVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 330 aa, synthetic construct
  source 1..330: note = Synthetic Construct
RECIYYNANWELERTNQSGLERCEGEQDKRLHCYASWRNSSGTIELVKKGCWLDDFNCYDRQECVATKENPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 330 aa, synthetic construct; identical to SEQ ID NO: 211
  source 1..330: note = Synthetic Construct

SEQ ID NO: 263 - protein, 337 aa, synthetic construct
  source 1..337: note = Synthetic Construct
GRGEAETRECIYYNANWELERTNQSGLERCEGEQRKRLHCYASWRNSSGTIEIVKQGCWLDDFNCYDRTDCVEKKDSPQVYFCCCEGNMCNEKFSYFPEMEVTQPTSGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 265 - protein, 4 aa, synthetic construct
TEEN

SEQ ID NO: 266 - protein, 4 aa, synthetic construct
TKEN

SEQ ID NO: 267 - protein, 4 aa, synthetic construct
KKDS

SEQ ID NO: 268 - protein, 4 aa, synthetic construct
TKDS

SEQ ID NO: 269 - protein, 115 aa, synthetic construct
  source 1..115: note = Synthetic Construct
  VARIANT 11: note = MISC_FEATURE - Xaa is Ile or Leu
  VARIANT 12: note = MISC_FEATURE - Xaa is Phe or Tyr
  VARIANT 19: note = MISC_FEATURE - Xaa is Leu or Lys
  VARIANT 20: note = MISC_FEATURE - Xaa is Asp or Glu
  VARIANT 25: note = MISC_FEATURE - Xaa is Thr or Ser
  VARIANT 27: note = MISC_FEATURE - Xaa is Leu or Val
  VARIANT 29: note = MISC_FEATURE - Xaa is Pro or Arg
  VARIANT 31: note = MISC_FEATURE - Xaa is Tyr or Glu
  VARIANT 33: note = MISC_FEATURE - Xaa is Asp or Glu
  VARIANT 34: note = MISC_FEATURE - Xaa is Lys or Gln
  VARIANT 38: note = MISC_FEATURE - Xaa is Arg or Leu
  VARIANT 41: note = MISC_FEATURE - Xaa is Tyr or Phe
  VARIANT 45: note = MISC_FEATURE - Xaa is Arg or Lys
  VARIANT 47: note = MISC_FEATURE - Xaa is Ser or Ile
  VARIANT 48: note = MISC_FEATURE - Xaa is Ser or Thr
  VARIANT 50: note = MISC_FEATURE - Xaa is Ser or Thr
  VARIANT 51: note = MISC_FEATURE - Xaa is Ile or Leu
  VARIANT 53: note = MISC_FEATURE - Xaa is Ile or Leu
  VARIANT 56: note = MISC_FEATURE - Xaa is Lys or Gln
  VARIANT 63: note = MISC_FEATURE - Xaa is Phe or Ile
  VARIANT 69: note = MISC_FEATURE - Xaa is Gln, Thr, or Asp
  VARIANT 70: note = MISC_FEATURE - Xaa is Glu, Asp, or Thr
  VARIANT 74: note = MISC_FEATURE - Xaa is Lys or Thr
  VARIANT 75: note = MISC_FEATURE - Xaa is Lys or Glu
  VARIANT 76: note = MISC_FEATURE - Xaa is Asp or Glu
  VARIANT 77: note = MISC_FEATURE - Xaa is Ser or Asn
  VARIANT 79: note = MISC_FEATURE - Xaa is Glu or Gln
  VARIANT 89: note = MISC_FEATURE - Xaa is Phe or Met
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 270 - protein, 115 aa, synthetic construct
  source 1..115: note = Synthetic Construct
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 271 - protein, 115 aa, synthetic construct
  source 1..115: note = Synthetic Construct
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 272 - protein, 115 aa, synthetic construct
  source 1..115: note = Synthetic Construct
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 273 - protein, 115 aa, synthetic construct
  source 1..115: note = Synthetic Construct
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 274 - protein, 115 aa, synthetic construct
  source 1..115: note = Synthetic Construct
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 275 - protein, 115 aa, synthetic construct
  source 1..115: note = Synthetic Construct
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 276 - protein, 115 aa, synthetic construct
  source 1..115: note = Synthetic Construct
GRGEAETRECIYYNANWELERTNQSGLERCYGDKDKRLHCYASWRNSSGTIELVKKGCWLDDFNCYDRQECVAKKDSPEVYFCCCEGNFCNERFTHLPEAGGPEVTYEPPPTAPT

SEQ ID NO: 277 - protein, 115 aa, synthetic construct
  source 1..115: note = Synthetic Construct
GRGEAETRECIYYNANWELERTNQSGLERCEGEQDKRLHCFASWKNSSGTIELVKQGCWLDDFNCYDRQECVAKKDSPEVYFCCCEGNFCNERFTHLPEAGGPEVTYEPPPTAPT

SEQ ID NO: 278 - protein, 115 aa, synthetic construct
  source 1..115: note = Synthetic Construct
GRGEAETRECIFYNANWEKDRTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 279 - protein, 115 aa, synthetic construct
  source 1..115: note = Synthetic Construct
GRGEAETRECIYYNANWELERTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 280 - protein, 115 aa, synthetic construct
  source 1..115: note = Synthetic Construct
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 281 - protein, 115 aa, synthetic construct
  source 1..115: note = Synthetic Construct
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 282 - protein, 115 aa, synthetic construct
  source 1..115: note = Synthetic Construct
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 283 - protein, 115 aa, synthetic construct
  source 1..115: note = Synthetic Construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 284 - protein, 345 aa, synthetic construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPTGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK